(19) United States
(12) Patent Application Publication
(10) Pub. No.: US 2025/0065248 A1
(43) Pub. Date: Feb. 27, 2025

(54) PROCESS FOR PURIFYING AND ENRICHING PROTEINS, NUCLEIC ACIDS OR VIRUSES USING AN AQUEOUS TWO-PHASE SYSTEM

(71) Applicant: Boehringer Ingelheim International GmbH, Ingelheim am Rhein (DE)

(72) Inventors: Michael Christian RICHTER, Ummendorf (DE); Frederik RUDOLPH, Biberach an der Riss (DE); Axel SCHMIDT, Clausthal-Zellerfeld (DE); Jochen STRUBE, Goettingen (DE)

(21) Appl. No.: 18/273,807

(22) PCT Filed: Jan. 24, 2022
(86) PCT No.: PCT/EP2022/051503

§ 371 Date: Jul. 31, 2024

(30) Foreign Application Priority Data
Jan. 25, 2021 (EP) 21153141.3

(51) Int. Cl. B01D 17/04; B01D 11/04; B01D 17/12; C12N 15/10; G01N 27/02

(52) U.S. Cl. CPC B01D 17/047; B01D 11/0492; B01D 17/12; C12N 15/1003; G01N 27/02

(57) ABSTRACT

The invention relates to a method for purifying and enriching a target product selected from
immunoglobulins or other proteins; or
plasmid DNA, genomic DNA, RNA or other nucleic acids or viruses, wherein the measured electrical conductivity values and/or the measured turbidity values of the phases are used for adjusting the position of the phases in a separating device (10, 100) and for separating the phases. The invention also relates to a device for carrying out the method and to the use thereof.

### RELATED APPLICATIONS

This application is a U.S. national stage entry under 35 U.S.C. § 371 of International Patent Application No. PCT/EP2022/051503, filed Jan. 24, 2022, which is hereby incorporated by reference in its entirety and which claims the benefit of priority to European Patent Application No. EP 21153141.3, filed Jan. 25, 2021.

### BACKGROUND OF THE INVENTION

### Technical Field

The present invention relates to methods for purifying and enriching immunoglobulins or other proteins; or plasmid DNA, genomic DNA, RNA or other nucleic acids; or viruses using an aqueous two-phase system.

### Prior Art

Biomolecules, such as proteins, have growing importance in a variety of applications, for example, as drugs, diagnostics, additives in food, detergents, as research reagents and the like. Extraction from natural sources is not sufficient, such that biotechnological production methods are used. This requires regular reprocessing and purification of the obtained protein.

For proteins used in biopharmaceuticals, such as therapeutic antibodies, not only product yield, but also the separation of impurities, is important. There are process-dependent impurities, such as host cells, components of host cells, e.g., proteins (host cell proteins, HCP) and nucleic acids resulting from the cell culture itself or from reprocessing, such as salts or detached chromatography ligands. In addition, product-dependent impurities may be present, such as molecular variants of the product with deviating properties, e.g., truncated forms, precursors, hydrolytic degradation products or modified forms, such as polymers and aggregates. Other impurities include contaminants, i.e., materials of a chemical, biochemical, or microbiological nature that are present in the cell culture in an undesirable manner.

Impurities in biopharmaceuticals are unacceptable, since they can cause undesirable side effects, such as allergic reactions or adverse immune reactions that could even lead to life-threatening anaphylactic shock. Therefore, there is a need for suitable cleaning methods that can reduce the undesirable impurities to a harmless minimum.

Monoclonal antibody production, as currently described in the prior art, is based on a so-called batch platform method [1,2]. Such method is divided into upstream and downstream processing. The production of the recombinant target protein is based on cell cultivation in bioreactors during upstream processing. The goal of subsequent downstream processing is to isolate target proteins from ancillary components, such as host cell proteins (HCPs), host cell DNA, media components, viruses, and endotoxins, using various unit operations, such as centrifugation, filtration and chromatography.

Such platform process comprises fed-batch suspension cultivation of mammalian cells in bioreactors with up to 20,000 L in volume, centrifugation and depth filtration as cell harvesting, protein A-affinity chromatography as capture (separation step), cation exchange chromatography (CIEX) as intermediate purification and hydrophobic interaction chromatography (HIC) as fine purification step. In addition, orthogonal virus inactivation steps are carried out with low pH and virus filtration to minimize immunogenicity. Following protein A affinity chromatography and due to the low pH for virus inactivation, diafiltration must be carried out in order to load the following CIEX.

However, with regard to established platform processes, chromatographic steps are reaching their capacity limits due to increasing product concentrations through upstream process optimization (i.e., cell engineering, media and process parameter optimization). This constraint is commonly known as a downstream bottleneck. As product concentration increases, the specific costs (€/kg) of upstream and downstream processing decrease. However, with higher product concentrations, the platform-dependent process reaches its optimum efficiency. An even greater increase of product concentration leads to a significant shift in cost of goods (COG) from upstream to downstream processing.

As such, there is a need for a suitable cleaning method that can be carried out in a simple manner and as economically as possible.

The current state of plasmid DNA (pDNA) production is also based on a batch platform process. During fermentation, pDNA is produced intracellularly in the E. coli system. As a rule, centrifugation of the fermentation solution is carried out for harvesting. The wet cell paste (WCP) obtained in this way is initially resuspended in the lysis method, then digested with an alkaline solution and neutralized by adding a potassium acetate buffer system. In the neutralization step, flocculation of proteins and other minor components occurs, assisted by the sodium dodecyl sulfate (SDS) used. The neutralized material is collected in a collecting container. There, the resulting flocs are partly separated by flotation, and at the bottom of the apparatus the solution is withdrawn through several fixed-bed layers and roughly clarified. Other steps in the reprocessing comprise diafiltration, concentration by ultrafiltration, precipitation by ammonium sulfate or other salts and two or more chromatography steps [3]. In the current method, the downstream bottleneck is the collection vessel of the neutralized material. In the case of an increase in throughput and/or scale-up, separation and clarification of the neutralized material are difficult due to the high biomass loading and simultaneous flocculation at the high volumes produced.

As such, there is also a need for a suitable cleaning method in this case. Since the stability of the pDNA is still low during such initial processing steps, as described above, the robust, timely, and simple isolation of the pDNA would be desired, as would the improved separation of impurities, which would stabilize the pDNA.

Process-dependent impurities, such as host cells, host cell components, e.g., proteins (host cell proteins, HCPs), nucleic acids, such as RNA and genomic DNA, endotoxins, membrane fragments and the like, which originate from the host cell itself and are released during fermentation or alkaline digestion, are also produced during pDNA production. The reprocessing results in, for example, salts or detached chromatography ligands or added substances necessary for the reprocessing, such as sodium dodecyl sulfate (SDS). In addition, product-dependent impurities such as isoforms, topoisomers, and oligomers of pDNA may be present with divergent properties. Other impurities include contaminants, i.e., materials of a chemical, biochemical or microbiological nature that are present in the fermentation broth in an undesirable manner. Also, like the previous processes, the production of viruses for gene and cell therapy is divided into an upstream and a downstream process. The upstream method involves the cultivation of the cells responsible for production and the transfection required for this purpose. The majority of viruses produced today can be classified as adenoviruses (average size 90 nm, non-enveloped), adeno-associated viruses (average size 20 nm, non-enveloped), and retro- and lentiviruses (average size 90-120 nm, enveloped). The presence of an additional lipid envelope in retroviruses and lentiviruses implies lower stability, which can decrease the overall yield after purification by up to 70%. The downstream method comprises the steps of cell harvesting and cell lysis in the case of a non-secreted product. Cell lysis is also often carried out during secretion to expose unsecreted product and thus maximize overall yield. Following these steps, the virus is present alongside a variety of impurities. In addition to media components from the cell culture, significant quantities of the vector used for transfection (usually pDNA) are usually present. In the case of cell lysis, intracellular components, such as genomic DNA and host cell proteins, are also released. In order to stabilize the product as quickly as possible and to isolate it from a large part of the minor components, a combination of cell harvesting/lysis and aqueous two-phase extraction is suitable, analogous to the explanations for the pDNA process. This can also replace the otherwise usually necessary step of a time-consuming and difficult-to-scale density gradient centrifugation. The further steps of purification to the final product, comprise for example dia- and ultrafiltrations for buffer exchange and concentration increase, along with chromatographies (size exclusion, affinity, ion exchange, hydrophobicity) [4]. As such, there is also a need for a suitable cleaning method in this case.

A well-known alternative to the adsorption-based and filtration-based techniques mentioned above is the gentle extraction of biomolecules, such as proteins, nucleic acids, cells, etc., using aqueous two-phase extraction (ATPE) [3].

An aqueous two-phase system (ATPS), also referred to here as a two-phase system for short, is an aqueous solution containing, for example, two immiscible polymers or a polymer and an inorganic or organic salt. After mixing the specified components, two phases are formed in the system. Due to different biophysical properties of the phases, it is possible to achieve a distribution of biomolecules into one of the two phases. Concentration and purification of the target molecule can thus be achieved in a single process step. For a higher purity of the target molecule, multi-step ATPS extractions can also be carried out.

As such, the formation of an ATPS (aqueous two-phase system) is achieved by combining certain phase-forming components in suitable concentrations. Known systems are obtained based on the following combinations:
polymer/polymer
polymer/salt
alcohol/salt
ionic fluids.

Polymer/salt two-phase systems (ATPS) are of particular interest for biotechnological uses. Polyethylene glycols (PEGs) with molecular weights between 200 and 4000 g/mol are frequently used as the polymer. Commonly used salts include sodium and potassium phosphates and citrates. The anion is dominantly responsible for phase formation and material properties compared to the cation. A polymer, such as PEG, is enriched in the upper light phase and the salt(s) is enriched in the lower heavy phase. The miscibility gap increases as the molar weight of the polymer increases. Part of process development is to identify the optimal system for the given process task.

In principle, therefore, the aqueous two-phase system (ATPS) serves to purify or enrich a biomolecule in that, after mixing of the aqueous two-phase system (ATPS), the biomolecule is enriched in one of the two aqueous phases, in particular is substantially located there, and the undesired impurities are enriched in the other phase, the counterphase, in particular are substantially located there, such that by separating off the counterphase containing the impurities the biomolecule is obtained with a significantly reduced proportion or proportion reduced to a minimum of impurities in one phase.

In the aqueous two-phase system (ATPS), it should be noted that the phases separate more slowly than usual aqueous/organic systems and emulsions are easily generated due to the low interfacial tensions and density differences [5]. The sometimes high viscosities (up to 40 mPas) of polyethylene glycol (PEG)-rich phases, which cause slower mass transfer and poor settling behavior, also make process prediction difficult.

In all devices for liquid-liquid extract, phase contact occurs if one phase is dispersed in the other. Thereby, each of the two phases can be dispersed in the other. This phase contact enables mass transfer, which can be carried out in one or more stages. The phases are usually separated in a separator or a mixer-separator. Single-stage mixer-separators are usually used in single-stage operation, while multiple mixer-separators connected in series can also work in so-called batteries in counterflow or crossflow operation. The most common extraction devices operating in counterflow mode are columns. For fluid phases with extremely small density difference, the Earth's gravity field alone is not sufficient to separate the phases in a reasonable time. Then, centrifuges are used to accelerate the liquid-liquid separation by applying centrifugal force through rotation of the centrifuge drum. These also allow separation of mixtures with extremely low density difference (Δρ less than 30 g/m³) in acceptable time [6].

A known method for the selective purification and enrichment of immunoglobulins or other proteins by means of an aqueous two-phase system using centrifugal force is known, for example, from WO 2014/135420 A1 [7]. According to one embodiment, the method comprises the following steps:
a. Providing a cell culture or cell culture supernatant containing the target protein;
b. Converting the cell culture or cell culture supernatant into an aqueous two-phase system by addition of a polymer and at least one salt, or two polymers in suitable concentration;
c. Mixing of the two-phase system to produce a dispersion;
d. Separation of heavy and light phase in a centrifugal extractor;
e. Recovery of the target protein from the light phase.

Separation using a centrifuge is disadvantageous, since large containers must be used and a lot of solvent must be used, which is disadvantageous in terms of poorer manageability along with economic aspects. In contrast to the disclosure of WO 2014/135420 A1 [7], it is not necessary to use a centrifugal extractor for the method of the present invention.

A method for selective purification and enrichment of plasmid DNA using an aqueous two-phase system has been reported, for example, from Frerix, A. et al [8]. The method is characterized as follows:
Alkaline cell lysis, precipitation after neutralization and extraction using a two-phase system (ATPS) with simultaneous clarification of the lysate takes place in one tank (one pot approach);
Two-phase system (ATPS), consisting of: PEG/salt (potassium phasphate and potassium citrate);
Strong enrichment of plasmid DNA in the heavy phase (bottom phase);
Genomic DNA, RNA in light phase (top phase) and/or intermediate phase (interphase).

In order to use aqueous two-phase extraction as a cell harvesting method, as in the present invention, it is necessary for the measurement equipment to meet the requirements for detection of a phase boundary interface or an additional intermediate phase formed. Detection of the two phases already poses a problem, since they have similar properties. An additional problem arises when an intermediate phase forms, which intermediate phase is an emulsion layer of cells, agglomerates and bioparticles that can contaminate the measurement equipment or affect the measurements.

A phase boundary interface is the boundary between two immiscible fluids. Thereby, the fluids are deposited on top of one another due to density differences. An additional intermediate phase, i.e., a third phase, can also form from the two immiscible phases, such that two phase boundary interfaces are then present. Detecting the phase boundary interface(s) or intermediate phase is useful if one wants to subtract only one of the two main phases, for example, when the target molecule is present in only one phase. Typically, the phase boundary interface(s) or intermediate phase can be seen in the transparent separators used for separation. Accurate detection of such phase boundary interface(s) or intermediate phase is essential for a smooth separation method. On the one hand, it ensures that the quality of the product does not change, and on the other hand, it increases process efficiency by reducing the frequency of shutdowns and allowing the full capacity of the equipment to be utilized. Optimal for accurate separation would be to provide an accurate profile of the phases over height (position of light phase, heavy phase, intermediate phase and phase boundary interface(s)) in particular at critical locations, in order to give the user accurate information for separation.

Numerous measuring devices are available on the market for phase boundary detection. These can be classified according to their general measuring principles and the mode of measurement: These are mechanical/optical measuring devices (e.g., float switches, sight glasses, displacers), electromechanical measuring devices (e.g., magneto-restrictive or capacitive detectors), electronic measuring devices with phase contact (e.g., radar, differential pressure, ultrasound) or electronic measuring devices without phase contact (e.g., nuclear measurement). Some of these will be briefly explained and their disadvantages will be described below:

A float switch is a mechanical/optical measurement method that can measure filling levels with the aid of buoyancy force, but can also be used to detect intermediate layers between two phases. Thereby, the weight of the switch must be adapted to the heavier fluid. Since the switch is a passive part, it does not have a mechanism for self-monitoring, which means that regular monitoring is necessary. The switch is also impaired in its operation by solids or excessively viscous fluids.

A sight glass as a mechanical/optical measuring device can be used to continuously measure the fluid level, and it is also possible to detect the phase boundary interface between two fluids. The sight glass is connected to the tank, such that if the intermediate layer is shifted too far, it is no longer detected by the sight glass. Solids may contaminate the glass, making visual detection impossible.

Another mechanical/optical measuring device is a displacer—filling level measuring device, which is placed in the fluid and experiences a buoyancy force depending on the fluid level. In a simple embodiment, a displacer is connected to a spring, for example. However, the measurement result strongly depends on the calibration at process conditions and an accumulation of solids can lead to falsification of the measurement.

With a magneto-restrictive measuring device, which uses an electromechanical measuring method, the interaction between two magnets is measured. One magnet is in the float, the other in a guide tube. The float can move freely up and down in its chamber as the filling level changes. A small pulse of current reaches the magnetic field of the float, causing the float to spin and emit a sound wave that can be measured. Usually, there is an indicator mounted on the outside of the chamber, such that one can see where the float is. Since the position of the float is detected, density changes will cause errors. Furthermore, solids can cause the float to get stuck.

Another measuring device uses an electromechanical method of capacitive measurement, with which an electrode is mounted in the tank. A tank wall is used as a second electrode or a reference electrode. If the fluid level increases, the material between the two electrodes changes, such that the capacitance of the capacitor changes. Disadvantages of this measuring system are that changes in the dielectric constant of the medium lead to errors. For tanks with non-conductive materials, a reference electrode must also be installed, making calibration difficult. Furthermore, the dielectric constant of the fluids with an intermediate layer measurement must be large enough to detect it and the upper medium must not be conductive with some measuring devices.

With electronic measuring devices with phase contact based on guided radar, a high-frequency radar pulse is guided along a probe. If the pulse hits a media surface, a portion of the pulses are returned and detected and evaluated by the device. The time that elapses between transmitting and receiving is a direct measure of the level of fluidity. The problem with this measuring method is that the medium with the lower dielectric constant, which must not change and should have a value below 10, must be on top; the dielectric constants of both phases must have a detectable difference, the top layer must be thick enough to be detected, and an existing emulsion layer can lead to measurement errors.

Differential pressure sensors are also known as electronic measuring devices with phase contact, which can measure an intermediate layer between two phases with different densities. The pressure difference is a direct measure of the filling level. To detect an intermediate layer, the filling level must be a certain height. The pressure difference must also be large enough, which is achieved either by a high density difference or a large apparatus setup. Density changes and a temperature dependence of the density of the fluids lead to errors.

Other electronic measuring devices with phase contact use ultrasound, wherein ultrasonic waves are emitted into the tank. This signal is sent back by fluids, which makes it possible to determine the filling level height and also intermediate layer detection due to the time difference. However, solids in the fluid cause the ultrasonic waves to scatter, which reduces the detected signal. Detectability depends strongly on the properties of the solids, since the signal can disappear completely if the proportion is too high. Furthermore, gases trapped in the fluid are a problem, since they also attenuate the signal.

Known electronic measuring devices without phase contact are based on a nuclear measurement, with which the attenuation of gamma radiation is detected. Thereby, weakening depends on the density of the medium. With the aid of such method, intermediate layers can also be detected. However, the measuring units are relatively expensive and require regular monitoring to guarantee safety. Furthermore, density changes that are too large and impurities on the tank wall can lead to errors.

The numerous disadvantages of the known measuring devices described above show that they are actually not suitable for the detection of one or even two phase boundary interfaces in the presence of an intermediate phase in an aqueous two-phase system. In some cases, the usual measurement techniques are not even applicable, such as detection by a sharp drop in capacitance (usually the organic phase), which is not possible in the aqueous two-phase systems due to the absence of an organic phase. Such measuring devices also reach their limits in some cases in large-scale production, wherein, for example, in the case of a sight glass in a separator, the large diameters required (DN600−1000) mean that reliable detection by sighting is no longer possible, despite the use of a transparent separator made of glass, due to the increasing turbidity.

However, it has been found that a suitable parameter for distinguishing the phases in aqueous two-phase systems is electrical conductivity. However, for this purpose a technique that allows the electrical conductivities of the two phases to be reliably distinguished must be developed and used.

From the prior art according to DE 44 00 397 A1 [9], a method for detecting the presence of one or more phases present in addition to a main phase has become known, wherein the introduction of the main phase into a separation vessel and then the measurement of the conductivity at the bottom and/or at the top of the separation vessel is performed. In this case, conductivity is only measured after separation into the individual phases, wherein measurements are only taken at the very top and bottom of the separation vessel. The measurement of conductivity is used solely for the purpose of demonstrating that multiple phases are present; conductivity is not used to separate the phases and remove them separately from a separating device.

DE 199 23 901 A1 [10] relates to a method for the controlled addition of emulsion breakers to a process tank, in particular to a separation tank filled with gas, oil, water and sand, wherein an emulsion layer is detected by means of a sensor with vertical spatial resolution and a sensor signal is passed to a feed control system, in order to add the quantity of emulsion breaker to be fed in a metered manner as a function of the sensor signal. Capacitive filling level sensors or gamma ray density profile sensors are particularly suitable as sensors.

KR 101 143 889 B1 [11] discloses an automatically locking fractionated hopper for improving methodability by installing an electrical conductivity sensor and automatically locking the hopper based on electrical signals. The hopper has a receiving part, an electrical conductivity sensor, a controller and a solenoid valve.

Furthermore, U.S. Pat. No. 3,220,930 A [12] discloses a device in which an emulsion of crude oils is broken by adding a chemical additive (so-called demulsifier). This is used to separate the water contained in the crude oil. For this purpose, a demulsifier is added to a water-containing crude oil emulsion in a tank, resulting in an oil phase, an oil/water intermediate phase and a water phase, each of which has different densities and different electrical properties. The demulsifier is, for example, Tretolite, Nalco 538, sodium hydroxide, sodium silicate or mixtures of these. An electrical probe 16 is used at the interface between the upper phase and the intermediate phase, and a probe for determining the density is placed at the interface between the intermediate phase and the lower phase. The electrical probe 16 is used to control the valve 22, which is used to control the quantity of demulsifier added. Thereby, only 2 states are distinguished: If the electrical probe 16 is in contact with the oil/water phase, current flows; when it is in contact with the oil phase, no current flows. The addition of a solid additive in the form of the demulsifier to the separating device complicates the control of the two-phase system, since the phases are first generated and then replicated to varying degrees, depending on the quantity of demulsifier. Control and monitoring simultaneously with an electrical probe and a density probe is highly complex and confusing. There is no aqueous two-phase system in which all phases have water as solvent.

EP 1 059 105 A2 [13] relates to a device and a method, in particular for separating water from a solvent mixture from textile cleaning. A combination of at least one gravity separator and, upstream thereof, at least one coalescence separator is provided, wherein a feed line of the gravity separator is connected to the outlet of the coalescence separator. In the coalescence separator, which is filled with an open-pored solvent-resistant foam, the volume of the water droplets is increased. A sensor 10 is provided in the gravity separator that, when a maximum height for the middle phase B is exceeded, sends a signal to a valve 12 and a pump 13 to return the solution to the coalescence separator. A further sensor 21 is provided in the lower part of the gravity separator. If the fluid level of phase C exceeds the sensor 21, the drain valve 20 is controlled accordingly. Both sensors are used for conductivity measurement or turbidity measurement. This is a classic recycling method that purifies a waste product that would otherwise have to be disposed of. The water is separated in order to be able to use the solvent mixture again. This is a very complex separation method. There is no aqueous two-phase system in which all phases have water as solvent.

In EP 1 762 616 A1 [14], a method for removing single-stranded nucleic acids from double-stranded nucleic acids is described by the following steps:
(a) Providing a mixture containing fully and/or partially double-stranded nucleic acids and optionally single-stranded nucleic acids;
(b) Resuspending the mixture specified in point (a) in an aqueous, low-molarity buffer system with low ionic strength and low buffer effect;
(c) Adjusting conditions in the mixture according to step (b) that result in reversible denaturation of a specific double-stranded nucleic acid or multiple specific double-stranded nucleic acids, wherein another nucleic acid or multiple other nucleic acids are irreversibly denatured;
(d) Further adding buffers and a polymer component to the mixture according to step (c);
(e) Incubating the mixture according to step (d) for a period of time sufficient to form an aqueous two-phase system having an upper phase and a lower phase; and
(f) Removing the upper phase containing single-stranded nucleic acid along with the interphase and collection of the double-stranded nucleic acid from the lower phase. According to examples 1 to 3, centrifugation is always used to separate the upper and lower phases. For the method of the present invention, no centrifugation, in particular centrifugal extraction, is used, since it interrupts the method in a detrimental way. Separation using a centrifuge also requires the use of large containers, wherein a large quantity of solvent must be used. This signifies poorer manageability and economic disadvantages.

Therefore, the present invention is based on the object of avoiding the disadvantages of the prior art and providing a method for purifying and enriching proteins, nucleic acids or viruses using an aqueous two-phase system that reliably enables the detection of the two phases and their transition in the form of the phase boundary interface or an optionally present intermediate phase (here, also called an intermediate layer) with two phase boundary interfaces. The method is also to enable the separation of phases from one another, wherein the target molecule in the form of a protein, nucleic acid or virus is enriched in one phase. The method is also to be continuously applicable. Large-scale industrial use of the method is also to be possible.

Surprisingly, it has now been found that in an aqueous two-phase system, the measured electrical conductivity in a separating device can be used to distinguish the phases present from one another, thus facilitating the separation of the phases. This is surprising because throughout the prior art, only organic solvent/water systems are ever used in conjunction with electrical conductivity measurement. This is because organic solvents do not conduct electric current, such that only 2 states need to be distinguished to identify the phases: no current flowing means an organic solvent or solvent mixture is present; current flowing means water or a water/solvent is measured. However, according to the invention, water is present as a solvent in all phases, such that it is completely unexpected that the measurement of electrical conductivity can serve as a parameter to distinguish between the individual water phases even in aqueous two-phase systems. An organic solvent is typically not present in the aqueous two-phase systems of the invention.

According to one embodiment, the method enables not only determining the presence of the two phases and distinguishing them, but also determining and detecting the exact position of the phase boundary interface or of any intermediate phase present between the phases to be separated, thus greatly facilitating and simplifying the separate removal of the individual phases from one another. As such, the present invention provides a method for conductivity-based control and monitoring of the position of the phase boundary interface(s), which is used for phase separation and the separate removal of the phases from a separating device.

### SUMMARY THE INVENTION

As such, the object described above is achieved according to the invention by providing a method for purifying and enriching a target product selected from
immunoglobulins or other proteins; or
plasmid DNA, genomic DNA, RNA or other nucleic acids; or
viruses,
   comprising the following steps:
a. Providing a starting solution containing the target product;
b. Converting the starting solution into an aqueous two-phase system by adding at least one polymer and at least one salt in a suitable concentration, or by adding at least two polymers in a suitable concentration;
c. Mixing the aqueous two-phase system while maintaining a mass transfer, such that the target product is enriched in the light or heavy phase;
d. Allowing the phases to segregate in a separating device into alight phase, a heavy phase and, if an intermediate phase is present, an intermediate phase;
e. Continuously measuring electrical conductivity in at least one of the phases, in particular in the light and/or heavy phase, in the separating device to determine the position of the two phases and the phase boundary interface or, in the presence of an intermediate phase, the position of the intermediate phase and the two phase boundary interfaces;
f. Removing the phases from the separating device based on the continuously measured electrical conductivity of at least one of the phases in step e., in particular the light and/or heavy phase; and
g. Extracting the phase with the target product.

As such, the present invention provides target product enrichment and separation and impurity removal simultaneously with the aid of an aqueous two-phase system. In steps a. to g. of the invention, the conventional use of separators, in particular centrifugators, filtration and chromatography, is completely dispensed with.

According to one embodiment of the invention, the method is used to purify and enrich a target product using as a starting solution a cell culture, a cell supernatant or a cell lysate, in which the target product is contained. In this case, the method of the present invention is used for cell harvesting and/or isolation of the target product from the cells, cell culture and/or cell lysate. The method according to the invention can therefore be used both for separating the cells and for processing the product produced by the cells, in which case the cells have already been separated. That is, the cells can be removed either beforehand, or only during the present method.

Instead of or in addition to electrical conductivity, turbidity can also be measured.

It is also an object of the invention to provide a device for carrying out the method of the invention, comprising
a separating device for separating the phases;
at least one outlet in the separating device;
at least one probe for measuring electrical conductivity and/or at least one probe for measuring turbidity in at least one of the phases in the separating device;
one or more removal device(s) and/or means for regulating and/or controlling the removal quantity, removal rate(s) and/or removal duration of the light phase, the heavy phase and, if present, the intermediate phase, based on the measured values of electrical conductivity (for the probe(s) for measuring electrical conductivity) or based on the measured values of turbidity (for the probe(s) for measuring turbidity) in at least one phase.

A removal device is, for example, an immersion tube, the structure and function of which will be explained in detail later.

The invention also relates to the use of the method or device according to the invention for adjusting the position of the phases in a separating device and separately removing the phases from a separating device based on the measured electrical conductivity values of the phases (for the probe(s) for measuring electrical conductivity) and/or based on the measured turbidity values of the phases (for the probe(s) for measuring turbidity).

Additional features and advantages of the disclosure will be indicated in the detailed description that follows and, in part, will be readily apparent to the person skilled in the prior art from the description or will be recognized by putting the disclosure as described herein into practice, including the detailed description that follows, the claims along with the accompanying drawings. The claims set forth below form a part of this description and are incorporated herein directly and by reference.

### BRIEF DESCRIPTION OF THE DRAWINGS

FIGS. 1 to 22 are schematic illustrations of exemplary embodiments of the present disclosure. In the figures in detail:

FIG. 1 shows a schematic side view in section of an exemplary embodiment of a separating device according to the invention, showing the course of an extraction of a batch at 3 points in time of the separation with a probe for measuring the electrical conductivity of the phases, plotted against time;

FIG. 2 shows a schematic side view in section of an exemplary embodiment of a separating device according to the invention, representing a continuous extraction with two probes for measuring the electrical conductivity of the phases;

FIG. 3 shows a schematic side view in section of an exemplary embodiment of a separating device according to the invention, representing a continuous extraction with two probes for measuring the electrical conductivity of the phases at a different point in time than FIG. 2;

FIG. 4 shows a schematic side view in section of a further exemplary embodiment of a separating device according to the invention, representing a continuous extraction with two probes for measuring the electrical conductivity of the phases;

FIG. 5 shows a cross-section of a further exemplary embodiment of a separating device according to the invention with 4 probes in a radial arrangement (left) and a side view thereof (right);

FIG. 6 shows a cross-section of a further exemplary embodiment of a separating device according to the invention with 4 probes in a radial arrangement with horizontal distribution (left) and a side view thereof (right);

FIG. 7a shows an illustration of the start-up of a continuous extraction of an exemplary embodiment of the method according to the invention in a separator in an aqueous two-phase phosphate system based on the measured electrical conductivity in [mS/cm] for the heavy phase with a first probe, plotted against time in [s];

FIG. 7b shows an illustration of the start-up of a continuous extraction of an exemplary embodiment of the method according to the invention in a separator in the aqueous two-phase phosphate system of FIG. 7a based on the measured electrical conductivity in [mS/cm] for the light phase with a second probe, plotted against time in [s];

FIG. 8 shows a course of a continuous extraction of an exemplary embodiment of the method according to the invention in a separator in an aqueous two-phase phosphate system, shown based on the electrical conductivity profiles measured with two probes in [mS/cm], in each case for the light and the heavy phase, plotted against time in [s];

FIG. 9a shows a course of a continuous extraction of an exemplary embodiment of the method according to the invention in a separator in an aqueous two-phase citrate system, shown based on the electrical conductivity profile in [mS/cm] for the heavy phase measured with a first probe, plotted against time in [s];

FIG. 9b shows a course of a continuous extraction of an exemplary embodiment of the method according to the invention in a separator in the aqueous two-phase citrate system of FIG. 9a, shown based on the electrical conductivity profile in [mS/cm] for the light phase measured with a second probe, plotted against time in [s];

FIG. 10 shows a course of a continuous extraction of an exemplary embodiment of the method, wherein a single-phase system is fed into the separator instead of a two-phase system;

FIG. 11 shows a course of a continuous extraction of an exemplary embodiment of the method, wherein the water content of the two-phase system is varied and the interference system is present in the separator from the start;

FIG. 12 shows a course of a continuous extraction of an exemplary embodiment of the method, wherein the polymer content of the two-phase system is varied and the interference system is present in the separating device from the start;

FIG. 13 shows a course of a continuous extraction of an exemplary embodiment of the method, wherein the buffer salt content of the two-phase system is varied and the interference system is present in the separating device from the start;

FIG. 14 shows a course of a continuous extraction of an exemplary embodiment of the method according to the invention in a separator in an aqueous two-phase citrate system with a cell culture, shown based on the electrical conductivity profiles measured with two probes in [mS/cm], in each case for the light and the heavy phase, plotted against time in [s];

FIG. 15 shows a bar graph showing the cell count in [10⁵ cells/mL] of the cell culture used in Example 5.1, along with the cell counts of the light phase at various points in time during the method;

FIG. 16 shows a course of a continuous extraction of an exemplary embodiment of the method not according to the invention in a separator in an aqueous two-phase phosphate system with a cell culture, shown based on the electrical conductivity profiles in [mS/cm] measured with two probes, in each case for the light and the heavy phase, plotted against time in [s];

FIG. 17 shows a course of a continuous extraction of an exemplary embodiment of the method according to the invention in a separator in an aqueous two-phase phosphate system with a cell culture, shown based on the electrical conductivity profiles measured with two probes in [mS/cm], in each case for the light and the heavy phase, plotted against time in [s];

FIG. 18 shows a bar graph showing the cell count in [10⁵ cells/mL] of the cell culture used in Examples 5.2 and 5.3, and the cell counts of the light and heavy phases at various points in time during the method;

FIG. 19A shows a course of a continuous extraction of an exemplary embodiment of the method according to the invention in a separator in an aqueous two-phase phosphate system with a cell culture, shown based on the electrical conductivity profiles measured with two probes in [mS/cm], in each case for the light and the heavy phase, plotted against time in [s];

FIG. 19B shows a course of a continuous extraction of an exemplary embodiment of the method according to the invention in a separator in an aqueous two-phase phosphate system with a cell culture, shown based on the height adjustment of the immersion tube in [cm] in the separator, which correlates with the conductivity values of FIG. 19A;

FIG. 20 shows a course of a continuous extraction of an exemplary embodiment of the method according to the invention in a separator in an aqueous two-phase citrate system with cell lysate, shown based on the electrical conductivity profiles measured with two probes in [mS/cm], in each case for the light and the heavy phase, plotted against time in [s];

FIG. 21 shows a bar graph showing the yield and concentration for plasmid DNA of the method described in Example 6;

FIG. 22 shows a course of a continuous extraction of an exemplary embodiment of the method according to the invention in a separator in an aqueous two-phase citrate system using two pumps, shown based on the pump flows during the method; and

FIG. 23 shows a course of a continuous extraction of an exemplary embodiment of the method according to the invention in a separator in an aqueous two-phase system using a probe for measuring the turbidity values.

### DETAILED DESCRIPTION OF THE INVENTION

### Definition of General Terms

Terms not specifically defined herein are to be given the meaning that a person skilled in the prior art would give them in light of the disclosure and context.

As used herein, “purification and enrichment” is understood to mean that undesired components, such as contaminants or impurities, in particular host cell proteins (HCPs), host cell DNA (DNA), media components, viruses and endotoxins and the like, can be depleted and the purity of the target product increases as a result of the method.

The term “enriching” also comprises concentrating, wherein “concentrating” in this connection means that the target product in step g. is obtained in a higher concentration (mass/volume) compared to step a.

“Conductivity” here is to be understood as electrical conductivity. When “measurement of the electrical conductivity of a phase” or similar wording is specified, it means that the electrical conductivity is measured at specific positions in that phase using one or more conductivity measuring devices, such as probes.

The term “conductivity probe” as used herein is to be understood as a probe that measures electrical conductivity.

The expression “electrical conductivity of the light/heavy phase” or similar expressions are to be understood to mean that the electrical conductivity of the light, heavy or intermediate phase is not actually measured, but the electrical conductivity at certain predefined locations or suitable positions in the separator is measured. The predefined locations or suitable positions are the locations in the separating device where one or more conductivity measuring devices, in particular one or more probes, measure the electrical conductivity.

“Turbidity” of a transparent fluid is an optical impression caused by small particles that have a refractive index different from the carrier medium or show absorption. The turbidity of a fluid is determined optically and measured with the aid of an electronic evaluation. For example, the wavelength of the measuring radiation is 860 nm in the infrared range (according to ISO 7027). Alternatively, turbidity can be measured with a white light lamp (e.g., a tungsten lamp) between 400 and 600 nm (EPA 180.1). The skilled person is familiar with the measurement of turbidity from the prior art, such that he can readily select and use a suitable measuring radiation. A distinction is made between two measuring methods: The weakening of the transmitted light radiation (so-called transmitted light) and the lateral scattering of the light radiation (so-called scattered light). In order to be able to compare measured turbidity values, the turbidity standard fluid formazine was created, such that, in practice, all turbidity units refer to its dilution. A common turbidity unit is FAU (formazine attenuation unit; transmitted light measurement; angle 0°; according to the provisions of the ISO 7027 standard). Turbidity can be measured by various measuring devices; these are turbidity measuring devices, in particular turbidity probes. Turbidity probes then measure, for example, light scattering (side scattering) or are transmitted light probes that measure the attenuation of transmitted light radiation. The measurement signal or the measurement unit here is absorbance. The wavelength of the measuring radiation can vary. For example, commercially available turbidity probes may be used.

The term “light phase probe” or similar terms refers to the probe that is immersed in the light phase where it measures electrical conductivity. Thus, such probe is assigned to the light phase. The “measurement of the electrical conductivity of the light phase” thus means the measurement of electrical conductivity with the probe assigned to the light phase.

The term “heavy phase probe” or similar terms refer to the probe that is immersed in the heavy phase where it measures the electrical conductivity. Such probe is thus assigned to the heavy phase. Thus, the “measurement of the electrical conductivity of the heavy phase” means the measurement of electrical conductivity with the probe assigned to the heavy phase.

The expression that a probe is inserted “at a suitable position” in the separating device means that the probe for the heavy phase is also immersed in it and the probe for the light phase is immersed in it, such that the respective probe can measure the electrical conductivity of the assigned phase. The probe measures electrical conductivity at the predefined location where it was placed in the phase.

“Regulating and/or controlling” a parameter is understood to mean the monitoring and correction and/or regulation of the parameter.

“Allowing to segregate” means that the aqueous phases of the two-phase system separate by themselves. Gravity is sufficient for this purpose.

The term “phase boundary interface(s)” means that either there is no intermediate phase, in which case only the light and heavy phases are present in the separating device and there is a phase boundary interface between them. Or there is an intermediate phase, in which case there is a phase boundary interface between the intermediate phase and the light phase and a phase boundary interface between the intermediate phase and the heavy phase. Therefore, the term “phase boundary(s)” comprises both variants.

The term “continuous” means that one or more material flows into the separating device and one or more material flows out of the separating device. Thereby, the sum of the incoming material flows is usually equal to the sum of the outgoing material flows. If a method or method step is carried out continuously, it is performed without interruption.

The term “discontinuous” means that processing is not continuous. For example, a quantity limited by the capacity of the separating device (one batch or formulation) can be fed and the product can be removed all at once after the method is complete. For example, individual products can also be manufactured initially (batch), then stored and methoded later.

The term “semi-continuous” means that continuous and discontinuous methods are present. If a method is semi-continuous, the method is only partially continuous. This means that the continuous method is interrupted. This procedure can then also be understood as discontinuous. If a method step is semi-continuous, this step comprises continuous and non-continuous sub-steps.

The term “polymer(s)” is understood here as such polymeric compounds that can form an aqueous two-phase system and/or can be used in an aqueous two-phase system, but do not adversely affect the target product as far as possible.

The term “salt(s)” refers to those salts that can form an aqueous two-phase system together with a polymer(s) and/or can be used in an aqueous two-phase system, but do not adversely affect the target product as far as possible. These are, for example, inorganic or organic salts, in particular buffer salts.

### EMBODIMENTS OF THE INVENTION

The method of the invention with its various embodiments will be explained in detail below.

In general, the method of the invention can be carried out continuously or discontinuously. If the method is carried out continuously, each step is carried out continuously and immediately (continuously) follows the next step.

Only individual method steps can also be carried out continuously.

The method can also be carried out discontinuously. The method can be discontinuous as a whole, for example when carried out in batches, or one or more steps of the method may be discontinuous.

In the method of the invention, steps carried out continuously and discontinuously (non-continuously) may also be combined.

A method step can comprise continuous and discontinuous substeps.

According to one embodiment, steps a) to g) or steps b) to g) or steps c) to g) or steps d) to g) are carried out continuously or discontinuously.

According to a further embodiment, steps a) to f) or steps b) to f) or steps c) to f) or steps d) to f) are carried out continuously or discontinuously.

The term “in continuous operation” as used here means that at least steps d., e. and f., which are carried out in the separating device, are each carried out continuously and that there is also no interruption between such steps, i.e., the partial method d. to f. is carried out continuously overall.

The method according to the invention for purifying and enriching a target product is suitable for any type of protein, in particular immunoglobulins, or for any type of nucleic acid, such as plasmid DNA, genomic DNA, RNA, or any type of virus, such as adenoviruses, adeno-associated viruses and retro- and lentiviruses, or other viruses not mentioned herein.

In step a. of the method of the invention, a starting solution containing the target product is initially provided. This can constitute any type of starting solution, as long as it can be converted to an aqueous two-phase system.

The term “starting solution” is to be understood as broadly as possible in this connection and comprises not only true solutions, but any type of homogeneous or even heterogeneous mixture with which liquid-liquid phase separation can be carried out. Thereby, dispersions are to be included. Therefore, the starting solution is a fluid-containing mixture of the components to be separated, which are dissolved or dispersed in water in the aqueous two-phase system. Therefore, the starting solution can be a true solution or a dispersion and can also have solid components, such as cells, cell fragments or precipitates dispersed in the solvent.

The starting solution can be a cell culture, a cell supernatant or a cell lysate containing the target product (e.g., antibodies, viruses, mRNA). In one embodiment, the starting solution is a cell lysate prepared by alkaline lysis of cells and contains plasmid DNA. Thereby, the cells can be prokaryotic or eukaryotic cells, in particular fungi, yeasts, bacteria, such as E. coli.

The starting solution is converted to an aqueous two-phase system (ATPS) in step b.; i.e., an aqueous two-phase system is prepared using the starting solution.

The preparation of the aqueous two-phase system can be carried out continuously, discontinuously or semi-continuously. For example, the two-phase system can be produced continuously. It is also possible to produce a batch of the two-phase system and then method it further.

To prepare an aqueous two-phase system, at least one polymer and at least one salt are added to the starting solution in a suitable concentration, or at least two polymers are added to the starting solution in a suitable concentration. The term “in a suitable concentration” means that such a concentration is used that an aqueous two-phase system is formed. This concentration depends on the starting materials used and is known to the person skilled in the prior art or can be determined by a few orienting tests.

The aqueous two-phase system is not particularly limited according to the invention, provided that a combination of polymer(s)/polymer(s) or polymer(s)/salt(s) is used. Of course, water is used as the solvent. That is, the solvent in all phases is water; an organic solvent is not present.

The polymer(s)/polymer(s) combination used is, for example, polyethylene glycols (PEGs) with different molecular weights and dextran. Dextrans may be used with different molecular weights dissolved in aqueous solution.

According to one embodiment of the invention, the polymer(s)/polymer(s) combination is, for example, selected from polyethylene glycol (PEG)/polyethylene glycol (PEG), such as PEG 400/PEG 8000, PEG400/PEG2000, polyethylene glycol (PEG)/dextran. Other phase-forming polymers, such as polyethylene glycols (PEG) include polypropylene glycols (PPG), dextrans, PEG-PPG-PEG copolymer (EOPO) and the like. The phase-forming polymers may be used with different molecular weights.

As the polymer(s)/salt(s) combination, polyethylene glycols (PEG) with different molecular weight and different salts, such as phosphate salts, ammonium salts, potassium salts, acetate salts, sodium salts or citrate salts, for example ammonium sulphate, potassium citrate, sodium citrate, potassium acetate, sodium acetate, ammonium acetate, potassium phosphate, are used. Thereby, multiple salts (combinations) can also be used at the same time. Other salts known in the prior art, which can form an aqueous two-phase system together with a polymer(s), may also be used.

According to another embodiment of the invention, the polymer(s)/salt(s) combination is selected from polyethylene glycol(s) (PEG)/phosphate salt(s) and optionally other salts. In the present invention, an aqueous two-phase system comprising at least one polymer and at least one phosphate salt is also referred to as a “two-phase phosphate system.”

According to another embodiment of the invention, the polymer(s)/salt(s) combination is selected from polyethylene glycol(s) (PEG)/citrate salt(s) and optionally other salts. In the present invention, an aqueous two-phase system comprising at least one polymer and at least one citrate salt is also referred to as a “two-phase citrate system.”

According to another embodiment of the invention, the polymer(s)/salt(s) combination is selected from polyethylene glycol(s) (PEG)/ammonium salt(s) and optionally other salts. In the present invention, an aqueous two-phase system comprising at least one polymer and at least one ammonium salt is also referred to as a “two-phase ammonium system.”

According to one embodiment of the invention, other phase-forming polymers may be used for the polymer(s)/salt(s) combination, for example selected from polyethylene glycols (PEG), polypropylene glycols (PPG), PEG-PPG-PEG copolymer (EOPO) and the like. The phase-forming polymers may be used with different molecular weights.

According to a further embodiment, the polymer used for the aqueous two-phase system is polyethylene glycol with a molecular weight between 200 and 8000 g/mol. In one embodiment, the polymer used is polyethylene glycol having a molecular weight between 200 and 6000 g/mol or between 200 and 4000 g/mol. Molecular weights other than those specifically disclosed for polyethylene glycol may also be used.

The polymer or polymers, such as polyethylene glycol, can be used, for example, in a concentration of 5 to 35 wt. % based on the total quantity of aqueous two-phase system.

The phosphate salts used are, for example, sodium and potassium phosphates and alkali hydrogen phosphates, such as sodium mono- or dihydrogen phosphate or potassium mono- or dihydrogen phosphate, and many others. Other usable salts are known to the skilled person. One or more phosphate salts may be present simultaneously.

The citrate salts used are, for example, sodium and potassium citrates and many others. Citric acid can also be used. One or more citrate salts may be present simultaneously.

The ammonium salts used are, for example, ammonium sulfate, ammonium chloride and many others. One or more ammonium salts may be present simultaneously.

Additionally, it may be advantageous if other salts are present in the polymer/salt combination, such as sodium chloride, potassium chloride, acetates, tris(hydroxymethyl)aminomethane (Tris). Acetic acid may also be used.

Biomolecule-stabilizing components, such as amino acids (e.g., L-arginine), or sugars (e.g., sorbitol), or additives, such as polysorbates, may also be added to the two-phase system to protect the target molecule from precipitation or product loss at high polymer or salt concentrations.

It has been found to be advantageous if a two-phase system is prepared with the starting solution, with which a phase ratio of the phase containing the target product to the counterphase of >1.1 is used. For example, a ratio (volume/volume) of phase to counterphase=1.11 or greater (for the phase) to 1.0 (for the counterphase) is advantageous. The phase ratio can be a volume ratio (volume/volume) or mass ratio (wt/wt). The separation of the protein, nucleic acid or virus can thus be improved.

According to a further embodiment, the density ratio of light and heavy phase in the two-phase system used is at least 1.06. Accordingly, the ratio of the density of the heavy phases to the density of the light phase is 1.06 or greater (for the heavy phase) to 1 (for the light phase). This signifies a density difference of at least 6% between the two phases; i.e., there can also be a very small density difference between the two phases and yet a separation of the phases can be carried out without any problems. Such a density ratio is advantageous with regard to a short method time.

Since aqueous two-phase extraction (ATPE) requires two immiscible phases for the separation of target product and impurities, one of the phases enriching the target product, in particular substantially containing it, and the counterphase enriching the impurities, in particular substantially containing them, it is expedient to select a corresponding aqueous two-phase system depending on the target product and impurities contained. This can be done by the skilled person according to the state of the art and his technical knowledge based on a few orienting tests.

After converting the starting solution into an aqueous two-phase system (ATPS) in step b., the mixing of the obtained aqueous two-phase system is carried out in step c. The mixing of the aqueous two-phase system is carried out while maintaining a mass transfer, with which the target product is enriched in the light or heavy phase, and in particular is now substantially in only one of the phases. The expression that the target product “is enriched in the light or heavy phase” means that approximately 50% or more or approximately 60% or more or approximately 70% or more or approximately 80% or more or approximately 85% or more or approximately 90% or more or approximately 95% or more or approximately 98% or more of target product is present in a phase. However, for unstable or easily precipitated products, as little as approximately 40% or more or approximately 45% or more of target product may be sufficient if it is enriched in one phase. This is particularly highly when dealing with expensive or very valuable target products.

The mixing of the aqueous two-phase system in step c. is carried out while maintaining mass transfer, in particular sufficient mass transfer. Mass transfer is not sufficient if the target product is not enriched in the light or heavy phase, as already defined. A suitable or sufficient mass transfer can be determined, for example, by preliminary tests or by analyzing the concentration of the target product in the individual phases after mixing.

Therefore, the mass transfer results from the different distribution in the phases. There are several ways for the skilled person to determine that mass transfer has occurred; i.e., that the target product has concentrated in one of the phases. The skilled person can determine this, for example, by simple preliminary tests. These are, for example, settling tests at defined energy inputs in a reaction vessel (batch mode). Therefore, the mass transfer can be influenced in particular by the energy input during mixing. The phase separation behavior of the aqueous two-phase system used and the content of the target product in the phases are also determined.

To investigate the phase separation behavior, for example, the energy input during mixing is varied and the separation time for the formation of the phases is determined. Thereby, the end of phase formation is usually if the height of the interface of the light phase/heavy phase in the reaction vessel no longer changes. In order to determine whether the target component has partially, mainly or completely transferred into the target phase and the energy input used leads to mass transfer, the content along with the process yield of the target component can be determined in the formed phases.

The energy input can be determined, for example, via a specific agitator speed in the selected test setup (defined type of agitator and defined reaction vessel (height/diameter)). A determined energy input for mass transfer of the target component in a given aqueous two-phase system can then be adapted accordingly, for example, for different extraction containers, process formats (batch and continuous phase separation) and agitator types and setups (active or stationary energy input).

Other influencing factors, such as temperature, concentration of target product and byproducts (such as host cell proteins, cell number), may be used to optimize the energy input for each two-phase system during the preliminary tests or settling tests.

It is also possible that the mass transfer in step c. under different conditions (among others: temperature, dwell time, agitator speed, energy input, structure of the mixer during active mixing, structure of a static mixer, flow rate, etc. in the static mixer during static mixing) can be determined directly in the device in which the method of the invention is carried out, and the best conditions for mass transfer according to the invention can be determined. For this purpose, the method is carried out, for example, under different conditions in step c. and the content of the different ingredients is determined in the settled and separated phases: e.g.: content of the target product in the target phase and optionally in the other phases.

This can be determined by different methods under the prior art. Examples include, among others: chromatographic methods, such as reversed phase HPLC and other HPLC methods (anion exchange, cation exchange, HIC (hxdrophobic interaction chromatography), affinity chromatography (protein A and other ligands, that can specifically bind the target product)), nano-HPLC, UHPLC, electrophoretic methods, such as SDS-PAGE (polyacrylamide gel electrophoresis) among others, antibody binding-based methods, such as ELISA, Western blot etc., fluorometric or spectroscopic methods for proteins providing corresponding spectroscopic and fluorometric signals, and all other methods under the prior art for the analysis of target products, such as proteins and nucleic acids, in particular quantitative analysis.

The content of nucleic acids, in particular plasmid DNA (pDNA) in the target phase can be determined by cation exchange HPLC, nano-HPLC, and UHPLC methods, spectroscopic chromogenic assays, agarose gel electrophoresis, capillary electrophoresis, and the like, among others.

It is also possible to determine the content of impurities not desired in the target phase in both the target phase and the other phase(s) by methods under the prior art. For example, host cell proteins can be determined by SDS-Page or ELISA, and DNA and RNA can be determined by methods commonly used and accepted in the pharmaceutical industry. Similarly, there are various methods under the prior art for determining the content of other impurities in the various phases, some of which are to be separated by ATPE: chromogenic assays and LAL (limulus amebocyte lysate test) for endotoxins, cation exchange HPLC for the determination of the different pDNA isoforms in the individual phases, quantitative determination methods for host DNA, RNA, host cell proteins, lipids, endotoxins, virus and viral components, etc.

In order to be able to determine a mass transfer, the content of the target product and/or the undesired impurities in the starting solution of step a. of the method according to the invention is compared with the content of the target product and/or the undesired impurities in the individual segregated phases in step d. Thereby, an enrichment or depletion of the target product and/or the undesired impurities in the respective phases is then determined. The content is the proportion, quantity or concentration of the target product or impurities in the respective phases. Therefore, a change in the content of the target product and/or impurities by comparing the starting solution of step a. with one, two, or all of the segregated phases in step d. indicates a mass transfer.

Mass transfer can be achieved by active and/or static mixing. With active mixing, the aqueous two-phase system (ATPS) is mixed by means of moving parts, such as an active stirrer. Active stirrers are, for example, rotating agitators or agitators with a different effective movement. Static mixing takes place without the aid of moving parts, for example in a static mixer. Known static mixer types are, for example, Kenics mixers, which consist of plates twisted by 180° with each helix offset by 90°; or Sulzer SMV mixers, constructed of corrugated slats that conduct the currents so that they cross one another. A mass transfer, in particular a sufficient mass transfer during mixing, can be achieved, for example, by the energy input or power input during mixing and/or also the duration or dwell time over which mixing takes place. This depends on the individual case.

In tests with a cell cultures, it was found that the turbidity of the light phase could be avoided if a power input of more than 30 kW/m³ and a dwell time of more than 5 minutes were used during mixing. Such exemplary values could serve as a guide for the skilled person. The skilled person can determine a suitable procedure based on a few orienting tests, wherein each material system leads to different values.

The mixing of the phases is necessary in any case to achieve redistribution of the target product to the desired phase. The mixing of the aqueous two-phase system, resulting in mass transfer, is useful to obtain the highest possible yield of the target product. Mixing can be recognized, for example, by the fact that the product-containing phase is continuous, i.e., does not form droplets, and the counterphase is disperse, i.e., forms droplets. Insufficient mixing can lead to incorrect phase composition or contamination of the target phase with the counterphase, resulting in reduced product purity or making separation of the target product impossible. An example of insufficient mixing with incorrect phase composition is explained in the exemplary embodiments.

In step d., the phases are allowed to segregate in a separating device into a light phase, a heavy phase and, if necessary, an intermediate phase. This can be performed continuously or discontinuously.

The separating device can be any type of separating device known to the skilled person that is suitable for liquid-liquid phase separation, such as a separator or mixer-separator.

With a continuous method, this is carried out in such a way that the feed and removal of the fluid takes place in such a way that separation into the individual phases and their separate removal takes place continuously. With a discontinuous method, a batch is fed into the separating device, the phases are allowed to segregate, and then a separation of the phases is carried out by means of separate removal from the separating device. In the case of a discontinuous method, allowing the phases to segregate can also be carried out directly in the device in which the mixing of the aqueous two-phase system (ATPS) takes place. Thereby, after the mixing is completed, the segregating of the phases is waited for. The device in which the mixing of the aqueous two-phase system (ATPS) takes place can thus also subsequently be used as a separating device. It is also possible to switch between discontinuous and continuous operation.

The upper phase in the aqueous two-phase system is the light phase and the lower phase is the heavy phase. The target product can be located in the light or heavy phase; this depends on the properties of the particular target product and the two-phase aqueous system selected.

According to one embodiment, the segregating can also be carried out semi-continuously. This means that the continuous method is briefly interrupted and the segregating in step d. is not carried out continuously, but is initially waited for. This can lead to a better formation of the phase boundary interface(s) and therefore facilitate the subsequent separation of the phases.

When providing the starting solution in step a., it can also be advantageous to method semi-continuously here, i.e., to interrupt the otherwise continuous method. For example, if plasmid DNA is to be purified and enriched as the target product, the cell lysate is prepared as the starting solution via alkaline lysis followed by neutralization, for example with citrate salt. Thus, the salt buffer (such as citrate) required for aqueous two-phase extraction (ATPE) is already available and the two-phase system can be prepared and used immediately. However, in order to accurately control lysis, it may be expedient if this is not carried out continuously, but rather a suitable duration of lysis is observed and only then is the continuous procedure resumed. The neutralization that is then required is carried out with the salt buffer that is also required for the aqueous two-phase extraction (ATPE) (in this example: citrate). Advantageously, the salt quantity required for aqueous two-phase extraction (APTE) can be added completely in the lysis step.

However, it may also be advantageous if for purification and enrichment, in particular concentration of plasmid DNA, alkaline lysis, neutralization, transfer to an aqueous two-phase system, allowing segregation and the separate removal of the phases are carried out continuously.

For example, it is also possible to proceed as follows: Alkaline lysis is performed continuously or discontinuously, and transfer into an aqueous two-phase system is performed continuously or discontinuously or in batches, for example also with alternating cycles in each case. For example, 2 separating devices could be used alternately with continuous and discontinuous operation, or 2 separating devices connected in series could be used.

In step e., a continuous measurement of electrical conductivity is carried out in the separating device. This can be used to monitor the course of allowing the phases to segregate. However, electrical conductivity is used in particular to determine the position of the two phases and the phase boundary interface or, in the presence of an intermediate phase, the position of the intermediate phase and the two phase boundary interfaces.

If a cell suspension or cell lysate is used as the starting solution, i.e., a dispersion of bioparticles comprising the target product, along with cells, cell fragments and medium, it is often the case that a mixed phase that is constructed of bioparticles, but that usually no longer contains the target product, is formed. This mixed phase is also referred to here as the “intermediate phase,” i.e., a phase that exists between the two phases to be separated. For example, the intermediate phase may be a mixture of a proportion of the heavy phase in the form of non-coalesced droplets and cells bound between them. The formation and size of the intermediate phase is substantially dependent on the cells. The intermediate phase can also consist of precipitate, which is produced, for example, during the recovery of plasmid DNA by the alkaline lysis of cells during neutralization. This precipitate contains, for example, precipitated potassium dodecyl sulfate (K-SDS), proteins, cell components, genomic DNA, RNA and the like.

It is obvious that phase separation is complicated by the formation of an intermediate phase. The intermediate phase could spread in a completely uncontrolled manner to an existing lower and/or upper device outlet during continuous phase separation in the separating device without a filling level control. Therefore, without monitoring or detection capability, the removal of the phases cannot be controlled and managed.

Electrical conductivity was selected as the material parameter to be measured for reliable phase detection in the present invention, since it was found that the differences of this in the two phases of an aqueous two-phase system (ATPS) are large enough to be detected. For example, in the polymer/salt system, the polymer or polymer phase has low electrical conductivity and the salt or salt-rich phase has high electrical conductivity. Since only two phases are formed in a polymer/salt ATPS at sufficiently high concentrations of polymer or salt (>binodal), there is always a sufficient difference in the electrical conductivities of the light and heavy phases. Similarly, correspondingly suitable polymer/polymer systems may also be used.

Since, to date, there is no measurement technique that meets the requirements for the detection of a phase boundary interface or intermediate phase of an aqueous two-phase system (ATPS), a concept initially had to be developed to determine electrical conductivity in a suitable manner.

Therefore, based on material property measurements, a conductivity-based measuring principle was developed and experimentally tested on ideal and real mixtures, wherein the target product can be either in the upper or lower phase. As it has been found, the measuring principle is suitable to determine the position of the individual phases or phase boundary interface(s) based on the measurement of electrical conductivity, and independent of the distribution of the target product and the impurities, since the conductivity difference of the phases to be separated are mostly not influenced by the target product and the impurities.

Controlled phase separation according to the invention, whether with or without an intermediate phase, using the continuously measured electrical conductivity values, increases the yield of the method along with the product purity and quality finally obtained.

According to one embodiment of the invention, continuous measurement of the electrical conductivity of the phases in the separating device is carried out using one or more conductivity measuring devices in the form of one, two, three or more probes. That is, depending on the embodiment, one, two or even more probes may be used simultaneously in the separating device. Conductivity probes are known to the skilled person from the prior art. A probe measures electrical conductivity at the lower end in the usual way by means of measuring contacts. Exemplary conductivity probes are 4-pole conductivity probes with 4 metal contacts. Other conductivity probes are also possible.

The probe(s) is (are) installed or recessed in the separating device, for example, with the aid of fittings, and connected to central measured value detection and control electronics.

Just one probe can be sufficient to measure electrical conductivity in the separator. If an extraction in batches is carried out, for example, only one conductivity probe can be used. This is arranged, for example, in the outlet of the separating device. The outlet is an opening in the lower part of the separating device and can be connected to or have a removal device for a controlled outlet, for example selected from a valve, flow regulator, a hose pinch clamp, a hose, a tube or a spigot and the like, or can have any other configuration. The conductivity probe, which is mounted, for example, in the lower region of the separating device, such as near the bottom, in the outlet, in particular in a hose, a tube, a spigot or the like connected to the outlet, but upstream of a valve or a hose pinch clamp or the like of the separating device, then detects the exiting phase based on its respective electrical conductivity during the emptying of the separating device, such that the phases can be separated from one another in a simple manner and collected in separate collection containers.

According to another embodiment, one or more probes are used for continuous measurement of the electrical conductivity of the heavy phase and one or more probes are used for continuous measurement of the electrical conductivity of the light phase at different positions in the separating device. It may be advantageous if more than one probe is used, since this allows the detection of the position of the phases and interfaces and their control to be more accurate and thus improved. For example, one probe can continuously measure the electrical conductivity of the heavy phase and one probe can continuously measure the electrical conductivity of the light phase.

If two probes are used and continuous extraction is carried out, it is advantageous to select the position of the two probes in a suitable manner. In the separating device, one outlet for the heavy phase can be provided in the lower part of the separating device (lower outlet) and one outlet for the light phase can be provided in the upper part of the separating device (upper outlet). Each of the two probes can then be assigned to a respective outlet, for example. The probe assigned to the respective phase can then be used to detect whether the corresponding phase is still present at the probe and thus in the vicinity of the respective outlet, or whether the phase boundary surface has already risen or sunk so far that the counterphase is present at the probe and thus in the vicinity of the outlet through which it is not to flow out.

The probe positions are then selected, for example, in such a way that—regardless of the distribution of the target product in the upper or lower phase—the probe for the light phase (upper phase) is positioned lower than the upper outlet. This allows the electrical conductivity values to detect an excessive increase of the heavy phase (lower phase) before it is withdrawn via the upper outlet. The probe for the heavy phase (lower phase) is expediently placed higher than the lower outlet. This allows the electrical conductivity values to detect an excessive decrease of the light phase (upper phase) before it is drained via the lower drain.

According to one embodiment, it is therefore advantageous if the probe for the light phase is positioned lower than the upper outlet in the separating device and the probe for the heavy phase is positioned higher than the lower outlet in the separating device.

Thereby, it is not necessary that the probes be positioned in the vicinity of the respective outlet, for example, directly above or directly below the respective outlet; rather, the respective height of the probes in the separating device relative to the outlets plays a role. Since the fluid height in the separating device is the same everywhere, “below an outlet” means that the probe is not at the same height level as the outlet, but is placed below the height of the discharge opening of the outlet. Similarly, “above an outlet” means that the probe is not at the same height level as the outlet, but is placed above the height of the discharge opening of the outlet. Therefore, for example, the height level of the fluid and the outlet under consideration is of importance.

According to a further embodiment, the probe(s) for the light phase are arranged higher than the probe(s) for the heavy phase, which is regularly the case, since the light phase is always located above the heavy phase. In other words, it is advantageous if the probe(s) for the light phase is (are) placed below the upper outlet but higher than the probe(s) for the heavy phase.

The advantage of such positioning of two probes in the separating device is that each of the two conductivity probes, which continuously measure electrical conductivity, immediately indicates the approach of the other phase or the intermediate phase by changing the conductivity value, and therefore exiting of the other or intermediate phase at the wrong outlet can be prevented. Regardless of whether the target product is in the light or heavy phase, preventing the removal of a phase and/or the intermediate phase at the wrong outlet can prevent product loss.

As mentioned above, more than 2 conductivity probes can also be provided in the separating device. For example, 3, 4, 5, 6, or more probes may be present in the separating device at the same time. A higher number of probes may be advantageous for more accurate detection of the location of individual phases and phase boundaries.

It has been found that when using two, three or more probes, different positions may be advantageous.

According to one embodiment, for continuous measurement of the electrical conductivity of the phases in the separating device, two, three, four or more probes are each inserted and introduced into an insertion opening in the wall of the separating device, wherein the insertion openings have an arrangement selected from:
an axial arrangement of the insertion openings, wherein the insertion openings are arranged on a straight connecting line;
a radial arrangement of the insertion openings, wherein the insertion openings are arranged in an arc; or
a radially offset arrangement of the insertion openings, wherein the insertion openings are arranged in an arc and additionally shifted in a preferred direction.

The arrangements described are of particular advantage. In particular, if a larger quantity of biomass is present and it is also unclear how large the intermediate phase will be formed, the described embodiments are advantageous. This makes it possible to better cover in particular intermediate spaces between the individual probes, for example, even if the phase boundaries shift during the method. It is also possible to be more flexible with multiple probes, in particular with the arrangements described. In addition, a large number of probes allows the immersion tube to be better positioned, such that phase separation can be optimized.

According to one embodiment, therefore, the insertion openings for the probes in the wall of the separating device may be in an axial arrangement; i.e., a connecting line between the individual insertion openings would result in a straight line or axis and a linear arrangement would result.

According to a further embodiment, the insertion openings for the probes in the wall of the separating device may also be in a radial arrangement. In the case of a radial arrangement of the insertion openings for the probes, these would be distributed at equal or different distances from one another on an imaginary arc, for example, on the round circumference of the separating device. A radial arrangement of the insertion openings and thus also of the probes present in the insertion openings is advantageous, since such a distribution enables a particularly advantageous detection and monitoring of the individual phases and thus a faster and more robust detection, control and monitoring of the method can take place.

According to a further embodiment, the insertion openings for the probes and thus, of course, the probes present in the insertion openings may also be present in the wall of the separating device in a radially offset arrangement. This means that the insertion openings and the probes are distributed at equal or different distances relative to one another on an arc and are also shifted to an equal or different extent in a preferred direction, for example the longitudinal axis of the separating device. A radially offset arrangement of the probes also leads to improved detection of the phases and faster and more robust method control and monitoring.

Mixed arrangements can also be used, wherein one or more insertion openings for the probes and thus also the probes are arranged partly radially and partly axially. Other arrangements for the insertion openings of the probes than those described above are also possible.

The number and arrangement or position of the probes in the separating device must be selected in each individual case and depends in particular on the target product and the impurities present along with the aqueous two-phase system used, and may also depend on whether or not an intermediate phase is formed.

Based on the continuously measured electrical conductivity, the position of the two phases and their phase boundary interface or alternatively the position of the two phases and an intermediate phase and their two phase boundary interfaces can be determined according to the invention. In order to determine exactly where the phases are in the separating device, reference is made, for example, to so-called target values. These are either already known or can be readily determined in preliminary tests.

In Table 1 below, exemplary electrical conductivities of light phase, heavy phase and intermediate phase are given for an aqueous two-phase citrate system and an aqueous two-phase phosphate system with and without cells:

TABLE 1
Exemplary electrical conductivities for an aqueous
two-phase citrate system and an aqueous two-phase
phosphate system with and without cells

| | Electrical | | Electrical | |
|---|---|---|---|---|
| | conductivity | | conductivity | |
| Phase | without cells | | with cells | |
| Light phase (phosphate) | 10-25 | mS/cm | 10-25 | mS/cm |
| Light phase (citrate) | 5-15 | mS/cm | 5-15 | mS/cm |
| Heavy phase (phosphate) | 60-80 | mS/cm | 60-80 | mS/cm |
| Heavy phase (citrate) | 30-50 | mS/cm | 30-50 | mS/cm |

| | | | |
|---|---|---|---|
| Intermediate phase (phosphate) | n.a. | 40-50 | mS/cm |
| Intermediate phase (citrate) | n.a. | 30-40 | mS/cm |

n.a. . . . not available, an intermediate phase does not occur, for example, if the target component or impurities (e.g., HCP, host cell DNA) precipitate in large quantities. The precipitation of the components from the solution, can occur, for example, at high salt contents, high polymer contents, corresponding pH values in the selected aqueous two-phase system for sensitive components.

As can be seen from Table 1 above, each phase has its own range of electrical conductivity. The exemplary values for electrical conductivity given in Table 1 can serve as target value ranges for the aqueous two-phase systems (ATPSs) used. For example, with an aqueous two-phase phosphate system, the range for electrical conductivity for the light phase can be in a target value range of 10-25 mS/cm and for the heavy phase can be in a target value range of 60-80 mS/cm. For example, the target value range for electrical conductivity for the light phase of an aqueous two-phase citrate system can be 5-15 mS/cm, and for the heavy phase, 30-50 mS/cm.

The ranges of values for electrical conductivity in the individual phases should not actually be changed by the presence of cells in the individual phases. However, in the presence of cells, an intermediate phase can occur, the electrical conductivity of which is in the range of 40-50 mS/cm in a two-phase phosphate system and in the range of 30-40 mS/cm in a two-phase citrate system.

The conductivity ranges given in Table 1 demonstrate that there is a sufficiently large difference between the individual phases in an aqueous two-phase system in order to enable the respective phases to be unambiguously determined based on their electrical conductivity values, such that phase separation is possible on the basis of the measured electrical conductivity.

The target values given in Table 1 depend on the respective polymer(s), the salt(s) used and the respective concentration, such that these target value ranges are primarily for orientation only and may also differ significantly from the specified ranges in individual cases. In particular, the presence of conductivity-lowering bioparticles (such as cells) can result in a significantly reduced electrical conductivity than would be expected.

For the skilled person, it is readily possible to select a two-phase system suitable for the purification and enrichment of a desired target product. Advantageously, there is a detectable difference in conductivity values between the phase to be separated with the target product and the counterphase or between the phase to be separated with the target product and the intermediate phase.

According to one embodiment, the differences in electrical conductivities from the phases to be separated are, for example, greater than 10 times, greater than 9 times, greater than 8 times, greater than 7 times, greater than 6 times, greater than 5 times, greater than 4 times or greater than 3 times the measurement accuracy of the conductivity measuring device. For example, if the electrical conductivity of the upper phase is 10 mS/cm and this results in an error for the measurement accuracy of 0.15 mS/cm, then 10 times the measurement accuracy means 10×0.15 mS/cm=1.5 mS/cm. Therefore, the difference in electrical conductivities between light phase and heavy phase would be greater than 1.5 mS/cm in this case.

Such a small difference in electrical conductivity between light and heavy phases can be present in polymer/polymer phase systems (ATPS).

For polymer/salt systems (ATPS), the rule of thumb is that the difference in electrical conductivities between the light and heavy phase is more than 2 mS/cm. However, such a difference is regularly present in all known polymer/salt ATPS systems, since the polymer phase with a low conductivity and the salt-rich phase with a high conductivity of the two phases are created by the high concentrations of polymer and salt, respectively, such that a sufficient difference in the electrical conductivities of the two phases is always obtained.

To determine where each phase is present in the separating device, the following procedure can be followed:

According to one embodiment, in a continuous system, the target value range for the light phase can be monitored and controlled as follows:

Continuous measurement of the electrical conductivity of the light phase in the separating device gives a value for electrical conductivity. Such value is compared with a target value range for electrical conductivity. The target value range is the range expected for the light phase. For example, the target value range for a light phase in an aqueous two-phase phosphate system is 10-25 mS/cm, as shown in Table 1. That is, the probe in the light phase measures electrical conductivity in the range of 10 to 25 mS/cm at the position where it is located. If the measurement of electrical conductivity now shows that this range is exceeded, i.e., the electrical conductivity is above 25 mS/cm or even significantly above, this means that the probe in the light phase starts to measure the higher electrical conductivity of an intermediate phase of 40-50 mS/cm or even the heavy phase of 60-80 mS/cm. The intermediate phase and possibly heavy phase therefore begin to build up in the separating device. Therefore, in order for the probe to be back in the light phase, the heavy phase and/or intermediate phase is therefore removed from the separating device; i.e., the removal rate of the heavy phase is increased in continuous operation, for example. This continues until the phase boundary moves downward and thus the measured conductivity of the light phase is again in the target value range. Similarly, the removal rate of the light phase from the separating device can also be reduced or stopped altogether, wherein the upper outlet is throttled or closed, for example.

According to another embodiment, in a continuous system, the target value range for the heavy phase can be monitored and controlled as follows:

Continuous measurement of the electrical conductivity of the heavy phase in the separating device gives a value for electrical conductivity. Such value is compared with a target value range for electrical conductivity. The target value range is the range to be expected for the heavy phase. For example, the target value range for a heavy phase in an aqueous two-phase phosphate system is 60-80 mS/cm. That is, the probe in the heavy phase measures electrical conductivity in the range of 60 to 80 mS/cm. If the measurement of electrical conductivity now shows that it falls below this range, i.e., below 60 mS/cm or even significantly below, this means that the probe in the heavy phase starts to measure the lower electrical conductivity of an intermediate phase of 40-50 mS/cm or even the light phase of 10-25 mS/cm. Therefore, the light phase or intermediate phase approaches the position of the heavy phase probe. For example, in order for the phase boundary interface to increase and the probe to be back in the heavy phase, the removal rate of the heavy phase from the separating device can be reduced. Alternatively, the removal of the heavy phase can be stopped altogether.

It is also possible to add an additional two-phase system, i.e., to increase the flow rate of the two-phase system fed, which again increases the fluid level in the separating device. This continues until the measured conductivity of the heavy phase is again within the target value range.

The control and monitoring of the removal of the phases from the separating device can be performed by the removal quantity, the removal rate and/or the removal duration, and the removal of a phase can also be completely interrupted. The person skilled in the prior art is familiar with measures and devices for this purpose. For example, the control and monitoring of the removal of the phases can be performed by gravity or using positive or negative pressure, for example a vacuum. Positive or negative pressure or a vacuum may be generated in any way. One or more pumps may also be used. Removal devices for changing the removal quantity are, for example, valves, flow limiters, hose pinch clamps and the like, which can also be used to completely stop or interrupt the removal of a phase. Removal devices for changing the removal rate are, for example, pumps. Measures and devices other than those described are also possible.

In addition, an immersion tube can also be used as an additional outlet for one phase. This embodiment of the invention will be explained in more detail.

By measuring the electrical conductivities of the phases, it is therefore possible to monitor and control both the decrease and increase of the individual phases and the phase boundary interfaces in the separating device. The increase of phases may be due to an accumulation of one or more phases or to the continuous feeding of further aqueous two-phase system to be separated. The decrease of the phases can be attributed to the removal of the phases. Therefore, in the continuous method, equilibrium is established between the fed and discharged phases, wherein the monitoring of electrical conductivity is used for this purpose. A very flexible monitoring and control system is therefore provided, which can intervene immediately in the event of phase (volume) changes in the separating device and also operates reliably during continuous extraction.

In step f., the phases are then separated based on the measured electrical conductivity by removing the phases from the separating device. The phases can be separated continuously or discontinuously by removing the respective phases from the separating device based on the measured electrical conductivity. The term “removing the respective phase based on the measured electrical conductivity” means that the respective phase to be separated can be identified based on or by its measured electrical conductivity values at one or more positions in the relevant phase and can therefore be selectively removed from the separating device. Therefore, the removal of the phase is monitored by measuring electrical conductivity. Therefore, the present invention links the known height of the measurement position along with the conductivity information of one or more probes to actively control the phase boundary in the separating device.

According to one embodiment, an outlet is provided in the separating device and the removal of at least one of the phases from the separating device through the outlet according to step f. is carried out by gravity or using positive or negative pressure, wherein a pump may be used. This can be the case, for example, with a discontinuously operated system. For example, if only one batch of material is to be separated via aqueous two-phase extraction, there can be only one outlet, which may be provided in the lower part of the separating device.

There may also be at least 2 outlets in the separating device; this may be the case, for example, in a continuously operated system. There may also be three or more outlets in the separating device.

In a continuous method, according to one embodiment, 2 outlets may be provided, an upper outlet located in the upper part of the separating device and a lower outlet located in the lower part of the separating device. An outlet is an opening through which the fluid exits. This can be designed in any way. The outlet can be closed continuously or in steps, for example by a valve, a flow limiter or a hose pinch clamp and the like.

According to a further embodiment, an upper outlet is provided in the separating device for removing the light phase from the separating device using gravity or positive pressure or negative pressure, wherein a pump can also be used to generate positive or negative pressure; and a lower outlet is provided in the separating device for removing the heavy phase from the separating device using gravity or positive pressure or negative pressure, where a pump can also be used to generate positive or negative pressure. This will be explained in more detail.

According to one embodiment, the removal of a phase according to step f. is performed in continuous operation by means of a height-adjustable immersion tube having an opening that is immersed in the phase to be removed, wherein
the immersion tube is connected to a lower outlet in such a way that the phase can flow out under gravity to the outlet through the opening in the immersion tube; or
the immersion tube has an opening (192) through which the removal of the phase is carried out using positive pressure or negative pressure, generated with or without a pump, or a pump.

According to this embodiment of the invention, an outlet can be configured to be a height-adjustable immersion tube, wherein an opening for removing a phase is provided in the immersion tube. An “immersion tube” is understood to be a tube that is inserted into the separating device from above and through an opening present in the immersion tube fluid of one phase can flow out or be withdrawn. The tube is immersed in the relevant phase in such a way that the phase can be removed from the separating device using the opening in the immersion tube. This can either be done passively, in that the phase exits by gravity, or this can be performed actively, in that pressure, in particular negative pressure, is applied to the immersion tube or the corresponding phase is pumped out by a pump connected to the immersion tube. The immersion tube is used to adjust the phase position(s) and position(s) of the phase boundary interface(s).

Therefore, the immersion tube represents an outlet present in the separating device. Thereby, the continuous or even discontinuous removal of the phases in the separating device according to step f. can be carried out via a height-adjustable immersion tube and thus a height-adjustable opening in the immersion tube.

The immersion tube can be used to remove the light phase, the heavy phase or the intermediate phase. The immersion tube can be used continuously or discontinuously. A semi-continuous mode of operation is also possible, wherein continuous sections alternate with discontinuous sections.

The immersion tube can be used, for example, to maintain a continuous method, wherein the adjusted equilibrium of fed and discharged phase(s) in the separating device is also maintained by use of the immersion tube. The immersion tube can also be used to eliminate temporary interferences, such as the build-up of one of the phases in the separating device, and then ensures that the built-up phase is allowed to flow out.

The immersion tube is immersed in one of the two or three phases in the separating device, by which the opening provided in the immersion tube allows this phase to be removed through the opening. Flowing out can take place simply by gravity; then, the immersion tube with opening is a type of overflow valve. However, the phase can also be actively removed through the opening in the immersion tube, for example using positive or negative pressure or a vacuum. A pump can also be used. If the immersion tube is operating in continuous operation using gravity as an overflow valve, it is expedient to have a lower outlet in the separating device open when the light phase is removed through the immersion tube. When removing the heavy phase through the immersion tube, it is expedient if an upper outlet in the separating device is opened.

The opening in the immersion tube can be permanently open; a closure for the opening, such as a lid or valve, could also be provided.

The height adjustability of the immersion tube means that the immersion tube can be moved up and down in the separating device and thus be immersed in one of the two or three phases, such that one of the phases is selectively removed with the immersion tube. In addition, the immersion tube can be moved up and down during extraction to respond to changes in the phases so that, for example, the opening in the immersion tube does not leave the phase and only the desired phase is removed through the existing opening.

“Driving up” or “moving up” the immersion tube is used interchangeably and means repositioning or readjusting the immersion tube upward while it is in operation, so that the immersion tube remains in the phase from which it is to remove phase fluid, and so that phase fluid can still be removed. The immersion tube can also be completely driven up or moved up. Then, for example, its opening is located above the fluid level or its opening is driven up until no more fluid can flow out of it. The immersion tube can be completely driven up, for example, at the start of the extraction method and at the end of the extraction method.

“Driving down” or “moving down” the immersion tube is used interchangeably and means positioning or readjusting the immersion tube downward while it is in operation, so that the immersion tube remains in the phase from which it is to remove phase fluid, and so that phase fluid can still be removed.

The expression that “the immersion tube is repositioned or readjusted upward or downward” means that the immersion tube is about to leave the relevant phase due to a change in the phase boundary interface(s) in the separating device, such that the height or immersion depth of the immersion tube must be adjusted accordingly in order to remain or reenter the relevant phase so that it can flow out or be withdrawn.

Thereby, the height adjustment of the immersion tube, for example with the aid of a stepper motor, can be controlled directly via the measured values of electrical conductivity. In continuous operation, the immersion tube is usually in constant use. If interferences occur in continuous operation, the operation of the immersion tube can be interrupted or the immersion tube can also be used first to allow the phase boundary interface(s) or the filling level to increase or decrease. In particular at the start of the extraction method (start-up) and at the end of the extraction method (shutdown), the immersion tube is usually not in operation; i.e., no fluid is removed with the opening in the immersion tube.

Thus, by removing one or more phases, a selective lowering of the phase boundary interface position and thus, of course, of the phase positions can be carried out.

The target product can be located in the light or heavy phase. Depending on the embodiment, the immersion tube can be used to remove the phase with or without a target product from the separating device.

The immersion tube can also be used to remove any intermediate phase from the separating device. If one of the phases and the intermediate phase are to be removed simultaneously, for example because the target product is in the other phase, two phases could also be removed from the separating device at the same time with the immersion tube. For this purpose, the opening of the immersion tube could be positioned at the phase boundary interface.

It has been found advantageous if the immersion tube is constructed from an inner tube comprising an opening, and the inner tube is surrounded by an outer tube in the form of a cladding tube or overtube. For example, the overtube is fixed and cannot be moved, whereas the inner tube can be moved up and down. However, the overtube can also be formed to be movable. The overtube, as well as the inner tube, can be made of any material as long as it does not adversely affect the fluids being removed. For example, stainless steel, plastic or glass are possible.

The overtube has an upper end and a lower end that define the length of the overtube. In general terms, the length of the overtube can be selected as desired. Depending on which phase is to be removed, the length of the overtube can be selected. In addition, an overtube extending from top to bottom or an overtube extending from bottom to top can be used.

In the case of an immersion tube with an overtube extending from top to bottom, it is expedient if the heavy phase or, if present, the intermediate phase is removed with such overtube. In this case, the upper end of the overtube is expediently located above the light phase, in particular above the fluid level (=the fluid level of the light phase) in the separating device; i.e., the overtube is mounted above the separating device, in particular above or on the upper wall of the separating device, for example the outer or inner side of the wall, and extends into the interior of the separating device. The lower end of the overtube extends (seen from above) into the heavy phase or, if present, into the intermediate phase, depending on which of the phases is to be removed.

According to one embodiment, the immersion tube therefore comprises an overtube extending from top to bottom, the upper end of which is located above the light phase and the lower end of which is located in the heavy phase.

In the case of an immersion tube with an overtube extending from bottom to top, it is expedient if the light phase or, if present, the intermediate phase is removed with such overtube. In this case, the lower end of the overtube is expediently located below the heavy phase in the separating device; i.e., the overtube is mounted below the separating device, in particular below or on the lower wall of the separating device, for example the outer or inner side of the bottom of the separating device, and extends into the interior of the separating device. The upper end of the overtube extends (as seen from below) into the light phase or, if present, into the intermediate phase, depending on which of the phases is to be removed.

According to one embodiment, the immersion tube therefore comprises an overtube extending from bottom to top, the upper end of which is located below the heavy phase and the upper end of which is located in the light phase.

In one embodiment, therefore, the height-adjustable immersion tube is formed from a height-adjustable inner tube and an overtube, wherein the overtube can extend from top to bottom or from bottom to top. The inner tube has an opening for the removal of the corresponding phase. The diameter of the overtube is larger than the diameter of the inner tube, such that an intermediate space is formed between the inner tube and the overtube. Therefore, the inner tube is surrounded by the overtube, such that an intermediate space is formed between the inner tube and the overtube.

In one embodiment, the immersion tube can be connected to the lower outlet such that the phase can flow out under gravity to the outlet through the opening in the immersion tube. If the immersion tube is now used with the inner tube and overtube as an overflow valve, and the inner tube extends from above the light phase fluid level to the bottom outlet, the overtube in the separating device is shorter than the inner tube, since the inner tube extends the full height of the separating device. The inner tube is then only partially inside the overtube and can be moved up and down there. Advantageously, the opening of the inner tube is moved only inside the overtube.

If the immersion tube with inner tube and overtube is used in such a way that the phase is actively removed, for example by applying negative pressure to the inner tube, then the overtube is a suction tube and it is longer than the inner tube in the separating device. The inner tube is located entirely inside the overtube and can be moved up and down there. Advantageously, the opening of the inner tube is moved only inside the overtube.

All embodiments of the immersion tube with an overtube have in common that, in each case, the overtube is formed so that it projects beyond the opening of the inner tube. The opening of the inner tube is thus located inside the overtube. The length of the overtube can be varied here depending on which phase is to be removed. How far the overtube projects beyond the opening of the inner tube depends on the individual case and can be determined and adjusted by the skilled person in a few tests.

The diameter of the overtube is adjusted to be larger than the diameter of the inner tube, such that an intermediate space is formed between the inner tube and the overtube. The phase into which the overtube extends flows into such intermediate space and can pass from the intermediate space to the opening in the inner tube, where the phase fluid can then exit. Therefore, the phase fluid does not reach the opening in the inner tube directly, but only via the intermediate space, such that the overtube provides inlet protection.

In the case of the overtube extending from top to bottom, for example, into the heavy phase, the heavy phase rises in the intermediate space and only then reaches the opening in the inner tube. Since the overtube extends beyond the opening of the inner tube, this ensures that any phase present above the lower end of the overtube, here the intermediate phase and the light phase, cannot readily reach the opening.

In the case of the overtube extending from top to bottom, for example, into the intermediate phase, the intermediate phase rises in the intermediate space and only then reaches the opening in the inner tube. Since the overtube extends beyond the opening of the inner tube, this ensures that a phase present above the lower end of the overtube, here the light phase, cannot readily reach the opening.

In the case of the overtube extending from the bottom to the top, for example, into the light phase, the light phase flows by gravity into the intermediate space between the inner tube and the overtube and only then reaches the opening in the inner tube. Since the overtube extends beyond the opening of the inner tube, this ensures that any phase present below the upper end of the overtube, here the intermediate phase and the heavy phase, cannot readily reach the opening. Therefore, the overtube extends the path of the undesired phase(s) to the opening.

In the case of the overtube extending from the bottom to the top, for example, into the intermediate phase, the intermediate phase flows by gravity into the intermediate space, which is filled, and only then reaches the opening in the inner tube. Since the overtube extends beyond the opening of the inner tube, this ensures that a phase present below the upper end of the overtube, here the heavy phase, cannot readily reach the opening.

The different variants of the overtube can each be combined with the embodiments of the inner tube, wherein a suitable design can be selected depending on which phase is to be removed and on where the target product is located.

The diameter of the overtube is only important insofar as the diameter of the overtube is larger than the diameter of the inner tube. Therefore, the diameters of the outer tube and inner tube are selected according to the embodiment. The respective diameters of the inner tube and the overtube and thus the size of the intermediate space formed do not play a major role, since the phase fluid will in any case rise up in the intermediate space or flow into it when the overtube is immersed in the phase. The desired rate of phase fluid inflow into the intermediate space, by varying the distance between the inner tube and the overtube to adjust a suitable size for the intermediate space to each application, can be optimized, for example, by a few orienting tests.

Therefore, the diameter of the overtube and thus the distance to the inner tube can be selected at will and selected by the person skilled in the prior art for the particular application. This amounts to for example: (for DN50) 10 mm for the inner tube and 20 mm for the overtube or (for DN150): 35 mm for the inner tube and 60 mm for the overtube.

If the overtube is not movable but rigid, it is expedient to determine prior to the start of removal which phase is to be removed with the immersion tube, since the length of the overtube determines which phase it is immersed in and the overtube cannot be easily replaced during operation.

If the overtube is formed to be movable, this can also be changed in length during removal if this becomes necessary.

Therefore, the overtube overhangs the opening in the inner tube, such that the overtube serves as additional inlet protection, so that only the phase into which the overtube extends can flow into the intermediate space between the inner and overtubes, and only this phase can also exit from the opening in the inner tube.

According to a further embodiment, the length of the overtube and its diameter are selected to prevent the entry of suspended matter, flocs and other solid or semi-solid components from the phase in which the overtube is immersed to the opening in the inner tube of the immersion tube. The overtube thus allows fluid to enter the opening of the inner tube, but shields the opening from suspended matter and larger components. This has the advantage that undesirable components, such as suspended matter, flocs and the like, are retained by the overtube and thus do not enter the opening to clog it.

According to one embodiment, the immersion tube functions as an overflow valve both with and without an overflow tube; i.e., the phase fluid can flow out of the opening under gravity. In this embodiment, the immersion tube is connected to the lower outlet with or without an overtube in such a way that fluid can flow out through the lower outlet only through the opening in the immersion tube, but fluid cannot flow directly from the separating device into the lower outlet. The immersion tube is connected to the lower outlet in such a way that there is a fluid-tight connection, but the immersion tube can still be moved up and down without impairing the connection to the outlet. Such connections are known to the skilled person and need not be explained in detail.

The immersion tube is in operation when fluid is removed or withdrawn from the separating device through the opening of the immersion tube. The immersion tube is not in operation if no fluid is removed or withdrawn from the separating device through the opening in the immersion tube.

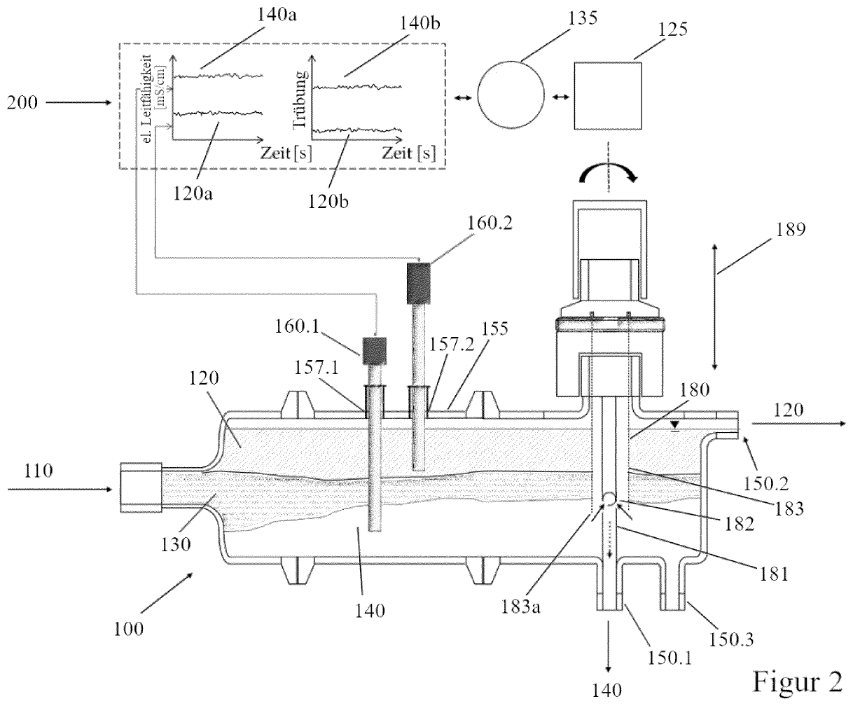

Based on the mobility of the inner tube, it could also be moved out of the overtube; however, this would eliminate the advantages of the overtube, so this is avoided. Details of the overtube are explained again at FIGS. 2 to 4.

According to another embodiment, the electrical conductivity values measured in step e. are used to control the immersion depth of the height-adjustable immersion tube through the immersion tube.

In a continuous extraction method, the continuous measurement of the electrical conductivities in the separating device, as described in step e., can therefore be used to control the height-adjustable immersion tube and regulate its height or immersion depth as a function of the position of the phase boundary interface and/or the phases, such that the desired phase can be removed through the opening present in the immersion tube. This can be performed in real time, such that the height adjustment of the immersion tube is performed automatically as a function of the measured values for electrical conductivity and thus the position of the respective phase and the position of the phase boundary interface(s). In addition, the discharge speed in the immersion tube can also be varied by the height adjustment, since the lower the opening of the immersion tube is in the respective phase, the faster the phase will discharge by gravity, since the hydrostatic pressure is higher.

Therefore, the immersion tube can be used in the continuous method, where it can be used continuously. However, it can also be used only temporarily and then eliminate the interferences that occur during continuous operation due to the accumulating phase over a limited period of use. The immersion tube is then reinserted if the interference develops and this is repeated until the method is terminated. The immersion tube would then be used discontinuously. Thereby, the respective phases flow out of the upper and, if applicable, lower outlet of the separating device, for example, driven by and according to the flow of the aqueous two-phase system (ATPS) into the separating device. The immersion tube can then serve as a fluid equalizer.

For example, there can also be 2 lower outlets in the separating device: one is connected to the immersion tube, and the heavy phase can be recovered from the separating device via the other.

In the present invention, the removal of the phases, and thus the general upward or downward shifting of the phase boundary interface(s) in the separating device, is monitored by one or more probes based on the continuously measured electrical conductivity values. Therefore, the present invention links the known height of the measurement position of the one or more probes and the conductivity information of the one or more probes to determine, actively control and be able to regulate the phase boundary(s) in the separating device in continuous operation. Thus, the phase boundary interface(s) can be readjusted upward or downward. This can be performed in a very general way by monitoring and controlling the aqueous two-phase system (ATPS) feeding to the separating device and/or the phases (that is, fluids, suspensions, precipitates and the like, in the form of the light, heavy and intermediate phases) discharging from the separating device. This can be performed, for example, by varying the feed quantity and/or feed rate of the incoming two-phase system (ATPS) (prepared with the starting solution) to the separating device and/or varying the removal quantity, removal rate and/or removal duration of light phase/heavy phase/intermediate phase from the separating device, and the removal of a phase can also be completely interrupted. This can be carried out in detail by
the removal rate for the heavy phase being increased or decreased;
the removal rate for the light phase being increased or decreased;
the removal quantity for the heavy phase being increased or decreased;
the removal quantity for the light phase being increased or decreased;
the removal duration for the heavy phase being increased or decreased;
the removal duration for the light phase being increased or decreased;
the removal of the light or heavy phase being interrupted;
the feed quantity of the aqueous two-phase system flowing into the separating device, with or without the target product, being increased or decreased;
the feed rate of the aqueous two-phase system (ATPS) flowing into the separating device, with or without a target product, being increased or decreased and/or
the immersion tube being used to allow to flow out or suck away a phase and being repositioned upward or downward if necessary.

The feed quantity and feed rate or removal quantity and removal rate are interrelated, such that, for example, the flow rate (volume per time period or quantity per time) can also be increased or decreased.

Varying the feed quantity/feed rate of the incoming aqueous two-phase system without the target product represents, for example, a flushing solution that can be used, in particular at the end of the extraction, to displace the light phase or the heavy phase from the separating device.

The individual measures described above can also be combined with one another. With the exception of the immersion tube described here, this is part of the knowledge of the skilled person.

The separating device may employ one or more removal devices and/or means for regulating and/or controlling the removal quantity, the removal rate and/or removal duration of light phase/heavy phase/intermediate phase from the separating device based on the measured values of the electrical conductivity values. These are known to the person skilled in the prior art, except for the use of an immersion tube, and comprise for example
the use of gravity in connection with one or more outlets along with the immersion tube: for example, allowing the heavy phase to flow out at the bottom outlet of the separating device or using the immersion tube as an additional outlet, which then acts as an overflow valve;
application of positive pressure in connection with an inlet, wherein the strength of the positive pressure can be controlled (driving force for the outflow of the phase(s) is positive pressure here): for example, feeding the aqueous two-phase system under pressure into the separating device;
application of a negative pressure in connection with one or more outlets along with the immersion tube, wherein the strength of the negative pressure can be controlled (driving force for the outflow of the phase(s) is here negative pressure): for example, at one or more outlets for drawing off the light and/or heavy phase; or with the immersion tube as an additional outlet, which then functions as a suction tube and sucks away one of the phases;
the use of one or more pumps in connection with one or more outlets along with the immersion tube, wherein their pumping speed can be controlled (driving force for the outflow of the phase(s) is here negative pressure): for example, for the feed of the two-phase system to the separating device and/or the removal at one or more outlets and/or through the immersion tube; and/or
the use of removal devices in connection with one or more outlets, which can regulate and/or control the cross-section of one or more outlets of the separating device, such as controllable valves, controllable flow limiters, controllable hose pinch clamps, controllable shut-off valves and the like (where the driving force for the outflow from the separating device can be gravity), each at one or more outlets of the separating device, for example for the controlled draining or interrupting of the removal of the phases at one or more outlets.

“Regulating and/or controlling the removal quantity, removal rate and/or removal duration” of the phases is understood to mean influencing the variation of the quantity, duration and/or speed of the fluid exiting the at least one outlet or the immersion tube. It involves monitoring and correcting and/or regulating the quantity, speed or duration of the exiting fluid.

In one embodiment, for example, based on the measured values of the electrical conductivity values, a height-adjustable immersion tube with an opening can be used to remove a phase using gravity (driving force for the outflow of the phase(s)). Thereby, the height of the opening in the separating device can be varied, as already described in detail.

The height of the opening of the immersion tube in the separating device can be adjusted continuously or in steps. Similarly, the removal from the separating device can also be stopped completely if the opening of the immersion tube is adjusted to be above the height of the light phase or the height of the phase fluid present in the intermediate space. Due to gravity and hydrostatic pressure, the lower the opening of the immersion tube in the separating device, the higher the removal rate from the separating device.

The one or more pumps used according to the invention may regulate the removal rate continuously or in steps. Likewise, regulation and/or control of the removal rate can be performed by switching the pump on and off (on/off control). Pumps can be feed pumps for conveying fluids or vacuum pumps that generate a pressure difference.

Negative pressure can be generated, for example, by compressing air. The strength of the negative pressure, which is applied to one or more outlets of the separating device, can also be adjusted continuously or in steps, or there is an on/off control. A vacuum can also be created with a pump.

The valves, flow regulators, hose pinch clamps, shut-off valves and the like can also be adjusted continuously or in step, such that the removal from the separating device can be controlled continuously or in steps. However, the valves, flow regulators, hose pinch clamps, shut-off valves can also regulate and/or control the outflow from the separating device by fully opening or closing it.

Such removal devices and means specified herein, including the immersion tube described above, by which the removal quantity, removal duration and/or removal rate of the phases from the separating device can be controlled and/or regulated, can also be combined, as the skilled person will readily understand.

For example, the heavy phase can be removed or withdrawn from the separating device via the immersion tube, and the removal quantity and/or removal rate of the light phase at an outlet can be regulated and/or controlled by a pump.

More generally, the removal of phases from the separating device can be both continuous and discontinuous, wherein the driving force for the outflow of phase fluid from the separating device is:
the force of gravity;
one or more pumps;
suction.

Furthermore, in the continuous method, the driving force for the outflow of the individual phases from the separating device is also the inflow of the aqueous two-phase system (ATPS) into the separating device. This has an influence on the removal rates of the light and heavy phases and, if applicable, the intermediate phase from the separating device. At equilibrium, the quantity of incoming aqueous two-phase system (ATPS)—the sum of the quantities of outgoing light, heavy and, if applicable, intermediate phase from the separating device. At equilibrium, this should actually also keep the phase boundary interface(s) constant in the center of the separating device. Fluctuations, interferences and the like can cause the phase boundary interface(s) to shift. In this case, a regulation is then undertaken.

According to one embodiment, the removal quantity of one or both phases and, if applicable, the intermediate phase from the separating device is controlled by:
the height of the opening of an immersion tube;
a pump the pumping speed of which can be controlled;
application of a negative pressure, wherein the strength of the negative pressure can be controlled;
one or more removal devices that can regulate and/or control the cross-section of one or both outlets of the separating device, such as adjustable valves, adjustable flow limiters, adjustable hose pinch clamps, adjustable shut-off valves and the like.

In addition to the measures and devices already described for controlling and monitoring the removal rate or quantity, in one embodiment the extraction speed of the fluid, for example of the light and/or heavy phase, is regulated or controlled by the adjusted pumping speed. For certain particularly sensitive target products, it may be advantageous to use pumps that enable low mechanical stress. This has the advantage that undesirable shear forces in the phases are avoided, making the method suitable for such target products.

The electrical conductivity values measured in step e. can thus be used to regulate and/or control the removal quantity, removal rate and/or removal duration of one or more phases, wherein the removal of a phase can also be interrupted entirely, for example by the removal devices and/or means described herein. In this way, the position of the phases and thus of the phase boundary interface(s) in the separating device can be selectively influenced.

For the position of the conductivity probes with regard to the position of the opening in the height-adjustable immersion tube, the above remarks on the position of probe/outlet may also be relevant here: that is, the opening in the immersion tube is nothing more than an outlet in the separating device.

In the embodiment without an overtube, if the immersion tube is immersed in the heavy phase to remove it, it can be expedient if the opening of the immersion tube for removing the heavy phase is located lower than the probe(s) in the heavy phase; i.e., the position where the probe measures the electrical conductivity is then located above the position where the opening of the immersion tube is located in the separating device. This makes it possible to detect an excessive decrease of the light phase (upper phase) and/or, if necessary, the intermediate phase with the electrical conductivity values before it flows out via the opening of the immersion tube.

In the embodiment without an overtube, if the immersion tube is immersed in the light phase to remove it, it is expedient if the opening of the immersion tube for removing the light phase is located higher than the probe(s) in the light phase; i.e., the position where the probe measures the electrical conductivity is located below the position where the opening of the immersion tube in the separating device is located. This makes it possible to detect an excessive increase of the heavy phase (lower phase) and/or, if necessary, the intermediate phase with the electrical conductivity values before it is withdrawn via the opening of the immersion tube.

According to another embodiment, when using an immersion tube with an overtube extending from top to bottom, when the heavy phase is removed through the opening of the inner tube, the lower end of the overtube for removing the heavy phase can be located lower than the probe(s) in the heavy phase; i.e., the position at which the probe measures electrical conductivity is above the position where the lower end of the overtube is located in the separating device. This makes it possible to detect an excessive decrease of the light phase (upper phase) and/or, if necessary, the intermediate phase with the electrical conductivity values before they enter the intermediate space between the inner tube and the overtube and flow out via the opening of the immersion tube.

According to another embodiment, when using an immersion tube with an overtube extending from the bottom to the top, when the light phase is removed through the opening of the immersion tube, the upper end of the overtube for removing the light phase can be located higher than the probe(s) in the light phase; i.e., the position at which the probe measures electrical conductivity is below the position where the upper end of the overtube is located in the separating device. This makes it possible to detect an excessive increase of the heavy phase (lower phase) and/or, if necessary, the intermediate phase with the electrical conductivity values before they enter intermediate the space between the inner tube and the overtube and flow out via the opening of the immersion tube.

Therefore, the present invention links the known height of the measurement position and the conductivity information of one or more probes to actively control the phase boundary in the separating device. In one possible embodiment, in the case of a horizontal separating device, such as a separator, with an immersion tube, for example in the form of a discharge tube or overflow valve, the phase boundary interface(s) would be readjusted upward or downward by automatically repositioning the immersion tube upward or downward to remove one of the phases.

Therefore, the control and monitoring method described, based on the measured conductivity values, can be used to ensure that one phase at a time is selectively removed only via one outlet at a time, e.g., the light phase only via the upper outlet and the heavy phase only via the lower outlet of the separating device or via the opening of the immersion tube with or without an overtube. Furthermore, the control and monitoring method can be used to perform a selective raising or lowering of the phase boundary interface(s). This is particularly advantageous during continuous extraction, since it prevents individual phases from accumulating or falling away.

In addition or as an alternative to measuring electrical conductivity, turbidity can also be measured and used as described for electrical conductivity.

The control and monitoring method can be carried out manually, automatically or using software.

In addition, selectively increasing the phase boundary interface position(s) in the separating device may be advantageous. This can be performed, for example, for the complete emptying of the separating device at the end of a continuous extraction, since otherwise a large part of a phase would possibly remain in the separating device and could not be recovered. For example, in the case of particularly valuable target products, it can be expedient to carry out a complete residual emptying of the separating device.

To enable the phase to be recovered as completely as possible and therefore the separating device to be emptied of residues, a flushing solution, for example composed of the heavy phase or light phase or another suitable solution, is added.

For example, by adding an appropriate flushing solution, the proportion of the heavy phase in the separating device can then be increased, such that the light phase is displaced upward and can thus be removed in a simple manner through the outlet arranged at the top. Alternatively, the proportion of the light phase in the separating device can be increased by adding an appropriate flushing solution in such a way that the heavy phase is displaced downward and can thus be removed in a simple manner through the outlet located at the bottom.

In another embodiment, the light phase can be displaced upward in the separating device by driving up the immersion tube that had removed heavy phase and therefore no further heavy phase is removed. This causes the heavy phase to accumulate and thus pushes the light phase upward, which can then be easily removed from the outlet.

In still another embodiment, the heavy phase can be displaced downward in the separating device by driving up the immersion tube that had removed light phase and therefore no further light phase is removed. This causes the light phase to accumulate and thus pushes the heavy phase down, which can then be easily removed from the outlet.

A “selective lowering or raising” means that this can be monitored in real time with the continuously measured conductivity values and controlled accordingly from the outside by the measures already explained in detail.

The desired phase with the target product is then obtained in step g.

The method of the invention can be carried out without intermediate steps, such that the individual steps a. to g. follow one another in each case.

It goes without saying that the method according to the invention can also be part of a large-scale industrial method. For example, the method according to the invention can be integrated as an automated cell harvesting and purification step (capture step) in the reprocessing, for example, of a monoclonal antibody as a target product on an industrial scale. In the first method stage, an upstream method, a cell culture is then cultivated in a bioreactor to produce the target product. In a second method stage, aqueous two-phase extraction is carried out according to the method according to the invention to purify and enrich the target product accordingly. In a third method stage, further purification steps for the target product could follow.

It is also an object of the invention to provide a method for the preparation of plasmid DNA, wherein
a plasmid DNA is provided;
this is transformed in a microorganism selected from eukaryotes and prokaryotes, propagated there and then recovered by alkaline lysis;
the method of the present invention is carried out;
subsequently, the plasmid DNA obtained is further purified, modified if necessary, and formulated if required.

In one embodiment, the method according to the invention can therefore also be used in the production of plasmid DNA on an industrial scale. Here, the plasmid DNA is produced intracellularly in a microorganism, such as in a eukaryotic or a prokaryotic organism, for example in yeast, in particular in a bacterium, for example in E. coli. Thereby, the microorganism is transformed with the plasmid DNA to be produced and the transformed microorganism is cultured under conditions that result in an increase of the quantity of plasmid DNA in the culture broth. After separation of the biomass (microorganisms containing plasmid DNA) and its alkaline lysis with subsequent neutralization, the lysate is transferred to an aqueous two-phase system (ATPS). A light and a heavy phase are formed and an intermediate phase containing precipitate of cell components, proteins, genomic DNA, RNA precipitated potassium dodecyl sulfate (and other impurities). Aqueous two-phase extraction (ATPE) according to the present invention purifies the plasmid DNA and enriches it in the heavy phase. The alkaline lysis and the immediately following aqueous two-phase extraction (ATPE) can be performed continuously and automatically. Additional steps in the reprocessing of the heavy phase following aqueous two-phase extraction (ATPE) comprise, for example, diafiltration, concentration by ultrafiltration, precipitation by ammonium sulfate or other salts, and/or two or more chromatography steps [3], for example, anion exchange chromatography and hydrophobic interaction chromatography. In further steps, the plasmid DNA can be concentrated to a specific concentration and ultradiafiltrated against a formulation buffer. After that, the plasmid DNA can be further formulated.

According to the invention, it is therefore possible to also carry out an automated phase separation, wherein a reliable detection of the two aqueous phases, without or with an intermediate phase, is carried out by continuous measurement(s) of the electrical conductivity at suitable position(s) in the separating device. Reliable detection of the phase boundary interface level, as achieved according to the invention, is necessary to provide a robust automation method. By automating the phase separation, based on the continuously measured conductivity values, a reliably operating system can thus be created.

The method according to the invention is also suitable for an industrial scale. Large-scale production with separating device volumes of more than 1 m³ is possible.

According to a further embodiment, the different turbidity of the different phases can be measured instead of or in addition to electrical conductivity. For example, electrical conductivity and/or turbidity can be continuously measured in at least one of the phases in the separating device.

The invention is then directed to a method for purifying and enriching a target product selected from
immunoglobulins or other proteins; or
plasmid DNA, genomic DNA, RNA or other nucleic acids; or
viruses,
comprising the following steps:
a. Providing a starting solution containing the target product;
b. Converting the starting solution into an aqueous two-phase system by adding at least one polymer and at least one salt in a suitable concentration, or by adding at least two polymers in a suitable concentration;
c. Mixing the aqueous two-phase system while maintaining a mass transfer, such that the target product is enriched in the light phase or in the heavy phase;
d. Allowing the phases to segregate in a separating device into alight phase, a heavy phase and, if an intermediate phase is present;
e. Continuously measuring electrical conductivity and/or turbidity in at least one of the phases in the separating device to determine the position of the two phases and the phase boundary interface, or in the presence of an intermediate phase, the position of the intermediate phase and the two phase boundary interfaces;
f. Removing each of the light phase, the heavy phase and, in the presence of an intermediate phase, the intermediate phase from the separating device based on the continuously measured conductivity and/or turbidity of at least one of the phases in step e.; and
g. Extracting the phase with the target product.

Turbidity can be measured with one or more turbidity measuring devices extending into the separating device in the form of one, two, three or more turbidity probes.

By way of example, the continuous measurement of turbidity in at least one of the phases in the separating device can be carried out with one or more turbidity measuring devices in the form of one, two, three or more probes, in particular, for continuous measurement of turbidity of the heavy phase at least one probe and for continuous measurement of turbidity of the light phase, at least one probe is used at a suitable position in the separating device.

Turbidity is measured optically and can be carried out with transmitted light measurement or scattered light measurement. According to the invention, a transmitted light measurement is preferably performed.

Turbidity is measured in FAU units (formazine attenuation units). However, other units for turbidity have become known (NTU, FTU) that could be used. As a rule of thumb, the difference in the measured turbidity values should preferably be approximately 100 FAU between the different phases, in order to distinguish the different phases from one another. In individual cases, however, lower values are also possible.

In the present disclosure, all embodiments are also to apply to the alternative or additional measurement of turbidity. In other words, the term “electrical conductivity” or comparable terms in the overall disclosure can each be replaced by or supplemented with the term “turbidity.” “Alternatively or additionally” means that electrical conductivity and/or turbidity are measured.

According to the invention, only electrical conductivity or only turbidity can be measured in a separating device. However, it is also possible that a combination of measurement of electrical conductivity and measurement of turbidity is used in a separating device according to the invention.

In the following, the invention will be explained and illustrated in detail with reference to the accompanying drawings, without limiting the invention herein. Reference is made in detail herein to various embodiments of the disclosure, which are illustrated in the accompanying drawings. Wherever possible, the same or similar reference marks and symbols are used throughout the drawings to refer to the same or similar parts. The various elements shown in the drawing are representative only and not necessarily drawn to scale. The drawings are intended to illustrate only exemplary embodiments of the disclosure of the invention, and a person skilled in the prior art will readily recognize where the drawings have been simplified to illustrate key aspects of the disclosure.

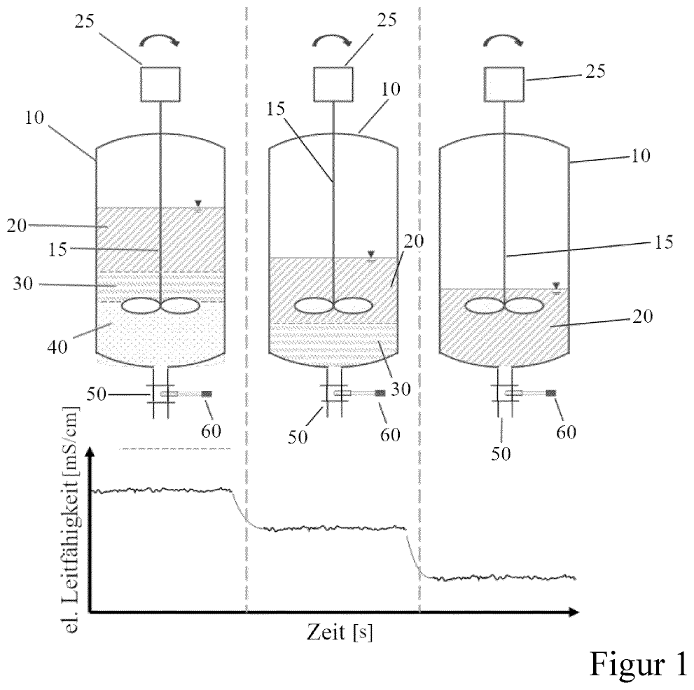

FIG. 1 is a schematic side view in section of an exemplary embodiment of a separating device 10 according to the invention, showing the course of an extraction of a batch (batch extraction) at 3 points in time of the separation with a probe 60 for measuring the electrical conductivity of the phases, plotted against time. In the example shown, the separating device 10 is a stirrer tank. This can have any suitable shape and size and be constructed of a suitable material. For example, a single-use bag vessel can also be used. The separating device 10 comprises an agitator, in this case an agitator shaft with a propeller agitator 15, which is driven by a motor 25. Other stirring devices are possible or can also be omitted depending on the application. The preparation and mixing of the aqueous two-phase system (ATPS) can be performed in this separating device 10 or outside the separating device 10, and the obtained aqueous two-phase system (ATPS) can then be transferred to the separating device 10. An outlet 50 is located at the bottom of the separating device 10. A probe 60 is mounted in the outlet 50 to measure the electrical conductivity of the fluid flowing past. In the graph below the 3 separating devices, the electrical conductivity in [mS/cm] of the respective phases measured continuously by probe 60 is plotted against time in [s].

The separating device 10 in the left image of FIG. 1 is already filled with all 3 phases. Accordingly, a starting solution has already been provided (step a.); this has been converted into an aqueous two-phase system in the separating device 10 by adding at least one polymer and at least one salt, or at least two polymers, in each case in suitable concentration (step b.) and has been mixed accordingly with the aid of the agitator 15 (step c.). In the example case shown, the target product is a protein that is enriched in, and in particular is substantially present in, the light phase. The phases obtained after the subsequent segregation of the phases (step d.) are illustrated in the left image of FIG. 1. The top phase 20 is the light phase containing the protein; the intermediate phase 30 and the bottom heavy phase 40 contain undesired components in the form of impurities and the like.

The three images shown in FIG. 1 illustrate the course of aqueous two-phase extraction of an exemplary protein (steps e., f. and g.) in an exemplary aqueous two-phase system for discontinuous or batch extraction in three steps:

The left image shows all three phases 20, 30, 40 in the separating device 10 after the phases have been segregated, wherein the heavy phase 40 is discharged from the outlet 50. The graph below the 3 separating devices 10 shows the electrical conductivity values measured by the probe 60 over the course of the extraction. For the separating device 10 in the left image, the graph indicates a high electrical conductivity for the heavy phase 40 measured with the probe 60, which remains constant for virtually the entire time that the heavy phase 40 is being drained from the separating device. Therefore, the heavy phase 40 can be unambiguously assigned by its electrical conductivity and thus distinguished from the other phases 30, 20 and thus precisely separated.

In the middle image of FIG. 1, the heavy phase 40 has already been separated and the intermediate phase 30 is now being drained from the outlet 50. The bottom graph now shows an average electrical conductivity for intermediate phase 30. Even when the intermediate phase 30 is drained from the separating device 10, the electrical conductivity measured with the probe 60 is nearly constant over time. The intermediate phase 30 shows different electrical conductivities than the light phase 20 or the heavy phase 40, such that an exact separation of the phases 20, 30, 40 from one another can be made based on the continuously measured conductivity.

In the right image of FIG. 1, the intermediate phase 30 has already been separated and the light phase 20 now remains in the separating device 10, which contains the target protein. Draining the light phase 20 from the outlet 50 indicates a low electrical conductivity for the light phase 20 measured with the probe 60 in the lower graph on the right, which remains nearly constant throughout the discharge of the light phase 20 from the outlet 50. Thus, the target protein in light phase 20 without the impurities is obtained.

Therefore, the separate draining of the phases can be controlled based on the measurement of the electrical conductivity: In this example case, with the target product in the light phase 20, the heavy phase 40 and the intermediate phase 30 are drained first and discarded if necessary. For example, as soon as conductivity falls below a certain threshold (target value range) after the intermediate phase 30 is drained, a valve provided below or downstream of the probe 60 (not shown) is automatically closed immediately. A line to a collection vessel or the like for the light phase 20 can be connected to the lower outlet 50 downstream or downstream of the valve, the valve can be opened again and the light phase 20 can be recovered in this way. Alternatively, a valve can be used to switch the flow from the separating device 10 from one line through which the heavy phase 40 and intermediate phase 30 are diverted to another line leading to a collection vessel or the like for the light phase 20. This line can also carry the light phase 20 directly to further processing, which can then be continuous, in batches or discontinuous.

Therefore, for discontinuous extraction or extraction in batches in a separating device 10 according to FIG. 1, phase detection with only a single probe 60 is possible, wherein phase separation can be carried out during emptying of the separating device 10, based on the continuously measured electrical conductivity.

FIG. 2 shows a schematic side view in section of an exemplary embodiment of a separating device 100 according to the invention, which represents a continuous extraction with two probes (160.1, 160.2) for measuring the electrical conductivity of the phases.

A starting solution has already been provided (step a.). In the example case shown, the starting solution is a cell culture. This has already been converted into an aqueous two-phase system by adding at least one polymer and at least one salt, or by adding at least two polymers, each in a suitable concentration (step b.) and mixed accordingly (step c.). The target product to be purified and enriched can be a protein, a nucleic acid or a virus. In the example case shown, the target product is a protein that is mostly in the light phase. However, it is also possible to purify and enrich a protein or nucleic acid that, for example, is enriched in the heavy phase after mixing and segregating of the phases, and in particular is substantially present in the heavy phase.

In the exemplary embodiment of the invention shown, the mixed aqueous two-phase system containing the target protein is transferred to the separating device 100. In the separating device, the phases are then segregated (step d.). The result is an upper light phase 120 in which the target product has been enriched, in particular is substantially present therein, for example in the form of a protein, an intermediate phase 130 and a lower heavy phase 140, in which the undesirable components have been enriched, in particular substantially have them.

The separating device 100 can be of any shape and size to carry out the phase separation. In the embodiment shown in FIG. 2, the separating device 100 is a type of side-lying separator that has three outlets 150.1, 150.2, and 150.3. Other shapes for the separating device 100 are possible.

Two probes 160.1 and 160.2 are provided in the separating device 100 to measure electrical conductivity of one phase at a time. The probe 160.1 is immersed in the heavy phase 140 and therefore measures the electrical conductivity of the heavy phase 140 in such position. The probe 160.2 is immersed in light phase 120 and therefore measures the electrical conductivity of the light phase 120 in such position. The probes 160.1 and 160.2 are respectively located in insertion openings 157.1 and 157.2, which are axially arranged in the wall 155 of the separating device 100, such that an axial arrangement results. The insertion openings 157.1 and 157.2, and thus also the probes 160.1 and 160.2 present therein, are arranged in the wall 155 of the separating device 100 such that they lie on a straight connecting line. In FIG. 2, the straight connecting line extends into the wall 155 of the separating device 100. Other embodiments are also possible.

The electrical conductivities measured with the probes 160.1 and 160.2 are shown schematically in a graph 200 above FIG. 2. Thus, the probe 160.1 continuously measures the electrical conductivity of the heavy phase 140, which is indicated in the curve 140a. The probe 160.2 continuously measures the electrical conductivity of the light phase 120, indicated by curve 120a.

The curves 120a and 140a in graph 200 show nearly constant values for electrical conductivity in the course of time; i.e., the two probes 160.1 and 160.2 are each in the phases that can be unambiguously assigned by their electrical conductivities.

In addition to the measured electrical conductivities in the left diagram of graph 200, the right diagram shows the measured turbidity of the phases plotted against time. Turbidity is measured in formazine attenuation units (FAUs) with a turbidity sensor. The curve 140b shows high turbidity of the heavy phase 140, since the heavy phase 140 is from a cell culture and therefore contains components that cause the high turbidity. The light phase 120b has low turbidity. Therefore, due to the high turbidity, phase separation is practically impossible with the measuring devices described in the prior art.

The separating device 100 is designed for a continuous mode of operation. Therefore, the solution 110 (=the aqueous two-phase system (ATPS)) to be separated can be fed in a constant flow (continuously) and the phases 120, 130 and 140 separated from one another can each be discharged continuously. The upper outlet 150.2 is used to remove the light phase 120; the lower outlet 150.1 or 150.3 is used to remove the heavy phase 140 and also the intermediate phase 130. The target product here is located in the light phase 120.

According to this exemplary embodiment of the invention, the immersion tube 180 is provided as an additional outlet. In the exemplary embodiment shown, it is constructed of an outer tube in the form of a cladding tube or overtube 183 and an inner tube 181. An opening 182 is located in the inner tube 181.

The overtube 183 has an upper end above the separating device 100, and its lower end 183a extends into the phase to be removed, here the heavy phase 140. For example, in the embodiment shown, the overtube 183 is, for example, not movable, but the inner tube 181 is. However, the overtube 183 could also be designed to be movable.

In the illustrated embodiment of FIG. 2, the diameter of the overtube 183 is shown to be larger than the diameter of the inner tube 181, such that the overtube 183 surrounds the inner tube 181 and only in an upper section of the inner tube 181. The opening 182 is located in this upper section of the inner tube 181, such that the overtube extends beyond the opening 182. Thus, the overtube 183 surrounds the height-adjustable inner tube 181 only in an upper section, wherein the length of the overtube 183 is selected such that the overtube 183 projects beyond the opening 182 of the inner tube 181.

This shields the opening 182 of the inner tube 181 from direct fluid access, here the heavy phase 140, from the separating device 100. The inner tube 181 is surrounded by the overtube 183 only in the upper section, where the opening 182 is located, but not in the lower section of the inner tube 181, such that the inner tube 181 is partially exposed in the heavy phase 140.

The overtube 183 and the inner tube 181 form an intermediate space into which the fluid of the phase in which the lower end 183a of the overtube 183 is immersed rises (not shown) and can then exit through the opening 182 in the inner tube 181. Thereby, the upward and downward movement of the opening 182 in the inner tube 181 can be illustrated as follows: The opening 182 can be positioned by moving it up and down to allow phase fluid rising in the intermediate space to flow out. If the opening 182 is positioned higher, the phase fluid can no longer flow out through the opening 182 and the immersion tube 180 is out of function. In addition, the position of the opening 182 determines how fast the phase fluid flows out due to the hydrostatic pressure.

Therefore, the phase fluid (here: heavy phase 140) must initially flow into the intermediate space between the inner tube 181 and then the overtube 183 before it can reach the opening 182 in the inner tube 181 (solid arrows in FIG. 2 pointing to the opening 182) and then flow out through it (dotted arrow in the inner tube 181 in FIG. 2).

The presence of an overtube 183, the end 183a of which is located in the phase 140 to be removed, is advantageous. The opening 182 in the inner tube 181 is shielded and protected by the overtube 183. The phase 140 to be removed rises into the intermediate space between the inner tube 181 and the overtube 183 and only then reaches the opening 182. Direct access of the fluid to the opening 182 is thus prevented. The overtube 183 thus increases the certainty that only the desired phase 140 can actually flow out.

By way of example, the length of the overtube 183 and its diameter, i.e., in particular the size of the intermediate space between the inner tube 181 and the overtube 183, can also be selected in such a way that the entry of suspended matter, flocs and other solid and semi-solid components to the opening 182 can be prevented so that no dogging of the opening 182 or the inner tube 181 occurs.

The skilled person can easily select and adjust the dimensions of the overtube 183 accordingly.

Other versions of the immersion tube 180 are also possible.

The inner tube 181 of the immersion tube 180 is connected to the outlet 150.1 in a fluid-tight manner, wherein, however, the inner tube 181 remains movable. The immersion tube 180 is thus still height-adjustable. The height adjustability or change in height is shown by the arrow 189 in FIG. 2 and is achieved by the motor 125, for example a stepper motor, which undertakes the height adjustment of the immersion tube 180 in the separating device 100. In the example case shown, height adjustability refers only to the inner tube 181, more specifically the opening 182 in the inner tube 181, which can be raised and lowered as required. The heavy phase 140 enters the intermediate space between the overtube 183 and the inner tube 181 and rises up in the overtube 181 to the opening 182 in the inner tube 181, where the fluid can then flow out of the separating device 100 into the outlet 150.1. The arrows at the opening 182 symbolize the flow of the heavy phase 140 into such intermediate space to the opening 182, and the dotted arrow in the inner tube 181 symbolizes the flowing out of such heavy phase 140 down through the inner tube 181 and out to the outlet 150.1. Therefore, the inner tube 181 is directly connected to the outlet 150.1. Other embodiments are also possible.

In the embodiment shown, the flowing out of the heavy phase 140 takes place with the aid of gravity alone.

In the illustrated embodiment, the end 183a of the overtube 183 is located within the heavy phase 140, such that only the heavy phase 140 can enter the intermediate space and flow out from the opening 182.

In FIG. 2, the probe 160.2 for the light phase 120 is positioned lower in the separating device 100 than the upper outlet 150.2. This is expedient so that the electrical conductivity is measured first and it is confirmed that only the light phase 120 is present and only then the light phase 120 exits the upper outlet 150.2.

In FIG. 2, the probe 160.1 for the heavy phase 140 is positioned lower than the lower end 183a of the overtube 183. However, it can also be advantageous for the probe 160.1 to be higher in the heavy phase 140 than the lower end 183a of the overtube 183. This can be used to detect excessive decrease of the light phase 120 and/or the intermediate phase 130 with the electrical conductivity values before they rise up into the intermediate space between the inner tube 181 and the overtube 183 and flow out through the opening 182 of the immersion tube 180.

In the described continuous extraction method according to FIG. 2, the continuous measurement of the electrical conductivities of the phases 120 and 140 according to step e. of the method according to the invention serves to control the height-adjustable immersion tube 180 and to regulate its height or immersion depth and, if necessary, also the duration of the removal as a function of the position of the phases or their phase boundary interfaces in such a way that the heavy phase 140 can flow out through the opening 182 present in the immersion tube 180. A controller 135 is used to automatically control the height adjustment of the immersion tube 180 as a function of the measured electrical conductivity values.

The regulation of the height-adjustable immersion tube 180 functions as follows: The respective probes 160.1 and 160.2 are each assigned target value ranges for the electrical conductivities of the phases resulting from the aqueous two-phase system used. As shown in Table 1 above, for example, an aqueous two-phase phosphate system has electrical conductivities in the range of 10-25 mS/cm for the light phase, and 10-25 mS/cm for the intermediate phase: 40-50 mS/cm and for the heavy phase: 60-80 mS/cm. Such target value ranges are used to determine and control where each phase is located in the separating device 100 with respect to the end 183a of the overtube 183.

The inner tube 181 of the immersion tube 183 is connected to the lower outlet 150.1 in the separating device 100, in order to allow the heavy phase 140 to flow out. In continuous operation, in FIG. 2, the heavy phase 140 can now be continuously removed with the immersion tube 180. Since the end 183a of the overtube 183 is located in the heavy phase 140, the heavy phase 140 rises into the intermediate space between the inner tube 181 and the overtube 183. In the embodiment shown in FIG. 2, therefore, there is always only the heavy phase 140 in the intermediate space between the inner tube 181 and the overtube 183 in continuous operation. As a result, the heavy phase 140 continuously flows out through the opening 182 of the inner tube 181 in continuous operation and exits the separating device 100 through the outlet 150.1. At the same time, the aqueous two-phase system (ATPS) 110 continuously flows into the separating device 100 and the light phase 120 continuously flows out of the separating device 100 via the upper outlet 150.2.

Ideally, there is an equilibrium between the quantity of fluid flowing in (aqueous two-phase system) and the quantity of fluid flowing out (the heavy phase 140 flows out via the immersion tube 180 and the light phase flows out via the outlet 150.2). The system runs continuously.

During continuous operation, if the phase boundary interface(s)/intermediate phase 130 shifts up or down, which is a regular occurrence, the inner tube 181 of the immersion tube 180, and thus the opening 182, can be adjusted, thereby controlling the removal rate of the heavy phase 140 from the separating device 100:

If the phase boundary interface(s)/intermediate phase 130 decreases, the value for the electrical conductivity of the probe 160.1 of the heavy phase 140 decreases. The opening 182 of the inner tube 181 is moved upward. A lower hydrostatic pressure is then present and the removal rate of the heavy phase 140 decreases.

Alternatively, the removal of the heavy phase 140 with the immersion tube 180 could be stopped altogether, for example by placing the opening 182 above the fluid level in the separating device 100. Then, the heavy phase 140 would accumulate again and the phase boundary interface(s)/intermediate phase 130 would increase again. Then, the opening 182 of the inner tube 181 could be returned to its original position if the value of electrical conductivity at the probe 160.1 for the heavy phase 140 is back within the target value range and indicates the expected conductivity value for the heavy phase 140.

If the probe 160.2 for the light phase 120 detects an increased value for electrical conductivity, the opening 182 of the inner tube 181 can be shifted downward. This results in a higher hydrostatic pressure. The removal rate of the heavy phase 140 increases. As a result, the phase boundary interface(n)/intermediate phase 130 decreases again. Thereafter, the opening 182 can be set back to its original position.

Therefore, in order to maintain equilibrium, there is a constant up and down movement of the immersion tube 180 (more precisely, the inner tube 181) during continuous operation, which corresponds to a typical zigzag movement. The immersion tube 180 is used continuously. Advantageously, the opening 182 of the inner tube 181 is moved up and down only within the overtube 183, in order to take advantage of the inlet protection of the overtube 183.

In an alternative embodiment, the immersion tube 180 can be used discontinuously. For example, in continuous operation, equilibrium can be established by the light phase 120 continuously flowing out of the upper outlet 150.2 and the heavy phase 140 out of the outlet 150.3.

If an interference of the equilibrium occurs here now, the immersion tube 180 can be used to compensate for the interferences. Thereby, the immersion tube 180 can be present with or without an overtube 183.

In this example case, a phase is not continuously removed through the immersion tube 180, but only in the event of an interference. The immersion tube 180 is again connected to the outlet 150.1 at the bottom of the separating device 100.

If the phase boundary interface(s)/intermediate phase 130 increases, the immersion tube 180 is moved from the initial position, for example, above the fluid level of the light phase 120 or above the fluid level in the intermediate space, for example, into the heavy phase 140, such that, in addition to the discharge from the opening 182 in the immersion tube 180, can also be discharged from the outlet 150.3. Thus, the overall outflow volume and speed of the heavy phase 140 from the separating device 100 increases and the phase boundary interface(s)/intermediate phase 130 decreases again. Then, the opening 182 of the immersion tube 180 can be moved back up to the initial position, for example, above the level of the light phase 120 or simply above the fluid level in the intermediate space.

In order for the phase boundary interface(n)/intermediate phase 130 to decrease, the immersion tube 180 can also be moved upward only in the heavy phase 140 and then the less heavy phase 130 flows out and the phase boundary interface(n)/intermediate phase 130 increases again.

If an overtube 183 is used for the immersion tube 180, the overtube 183 may or may not be variable in length. If the length of the overtube 183 is not variable during operation, it is expedient to select the corresponding length of the overtube 183 before starting the extraction. Depending on the desired embodiment, the overtube 183 can then be selected in the appropriate size and dimensioned to extend into the relevant phase. If a variable length overtube 183 is used, its length can be suitably changed during operation. The skilled person can readily implement this.

The overtube 183 prevents in particular the entry of the undesired phase(s) (here: the intermediate phase 130 and/or the light phase 120), provided that the phase boundary interface(s) do not decrease too far.

Therefore, the immersion tube 180 is used during continuous operation of the separating device 100 either in a continuous or discontinuous manner to restore the equilibrium between the fed and discharged phases.

In the example case shown in FIG. 2, the height-adjustable immersion tube 180 with an overtube 183 extending from top to bottom therefore serves as an outlet for the heavy phase 140.

The target product, here a protein, is obtained in the light phase 120 removed through the outlet 150.2 in the embodiment shown of FIG. 2 (step g. of the method).

In an alternative embodiment to FIG. 2, instead of extending from top to bottom, the overtube 183 could extend from bottom to top (not shown) and then extend into the light phase 120, wherein all other components in FIG. 2 are able to be taken over identically.

In this embodiment, the immersion tube would serve as an outlet for the light phase 120. Thereby, the light phase 120 initially flows into the intermediate space between the inner tube 181 and the overtube, and the intermediate phase fills from bottom to top only with light phase 120 until it arrives at the opening 182 in the inner tube 181, where it can then flow out into the outlet 150.1.

In this embodiment (not shown), it would be expedient for the probe 160.2 in the light phase 120 to be positioned lower than the upper end of the overtube extending from bottom to top. This would allow the electrical conductivity values to detect an excessive increase of the heavy phase 140 (lower phase) and/or, if applicable, the intermediate phase 130, before they could flow into the intermediate space between the inner tube 181 and the overtube and reach the opening 182 of the immersion tube.

An overtube extending from the bottom to the top (not shown) would prevent the entry of undesired phase (here: intermediate phase and/or heavy phase), provided the phase boundary interface(s) do not increase too far.

Therefore, the above remarks on FIG. 2 apply analogously to this embodiment with the overtube extending from bottom to top.

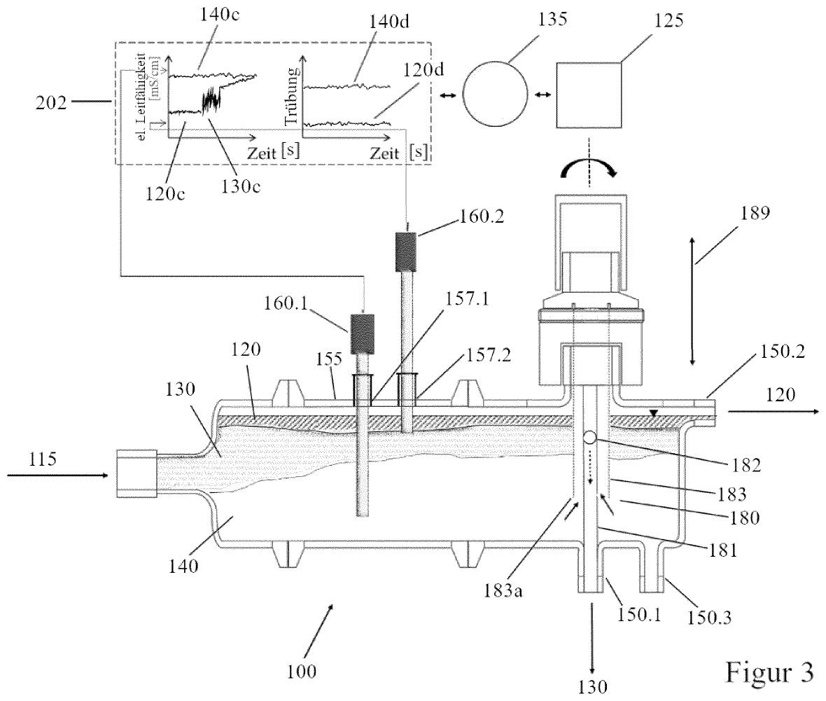

FIG. 3 shows a schematic side view in section of a further exemplary embodiment of a separating device 100 according to the invention, representing a continuous extraction with two probes (160.1, 160.2) for measuring the electrical conductivity of the phases at a point in time different from FIG. 2.

FIG. 3 illustrates how, by selectively increasing or raising the phase boundary interface position in the separating device 100, a majority of the light phase 120 that would otherwise remain in the separating device 100 can be recovered. To enable the light phase 120 to be recovered as completely as possible, the remaining light phase 120 is displaced upward by flushing with a flushing solution 115, for example by a proportion of the aqueous two-phase system used.

The selective increasing or raising of the fluid level in the separating device 100 is achieved, for example, by adding more heavy phase 140 to the separating device 100, such that the light phase 120 is forced upward and can thus be easily removed through the outlet 150.2. The displacement of the light phase 120 can also be achieved in a continuous extraction by driving up the immersion tube 180, thereby removing any heavy phase 140 and allowing it to accumulate in the separating device 100, thereby displacing the light phase 120 upward.

Analogous to the above procedures, heavy phase 140 could also be displaced downward.

The shifting of the phase boundary interface, if no intermediate phase is present or—as in the example case shown—if an intermediate phase is present, the shifting of the entire intermediate phase 130 and the heavy phase 140 upward in the separating device 100, is again monitored based on the continuously measured values for the electrical conductivity by the 2 probes 160.1 and 160.2.

Alternatively, it can also be expedient to selectively shift downward the phase boundary interface between the upper and lower phases, or even any intermediate phase that may be present (and thus also the two phases). This can be achieved, for example, as already explained, by the selective draining or removal of the corresponding phase(s).

In FIG. 3, insertion openings 157.1 and 157.2 are provided in the wall 155 of the separating device 100, wherein the probe 160.1 is present in the insertion opening 157.1 and the probe 160.2 is present in the insertion opening 157.2. Thereby, the insertion openings 157.1 and 157.2 and thus also the probes 160.1 and 160.2 are arranged axially in the wall 155 of the separating device; i.e., the insertion openings 157.1 and 157.2 are arranged in the wall 155 of the separating device 100 in such a way that they lie on a straight connecting line. In FIG. 3, the straight connecting line extends into the wall 155 of the separating device 100.

The measured electrical conductivities are shown schematically in graph 202 above FIG. 3. One can see from this the course of the continuous extraction, wherein the curve for the electrical conductivity of the light phase 120c initially remains constant until the probe 160.2 encounters the intermediate phase 130, shown with the curve 130c. The probe 160.1 detects the electrical conductivity of the heavy phase 140, which remains constant, with the curve 140c. The curve 140d and 120d again show the turbidity of the heavy phase 140 and the light phase 120.

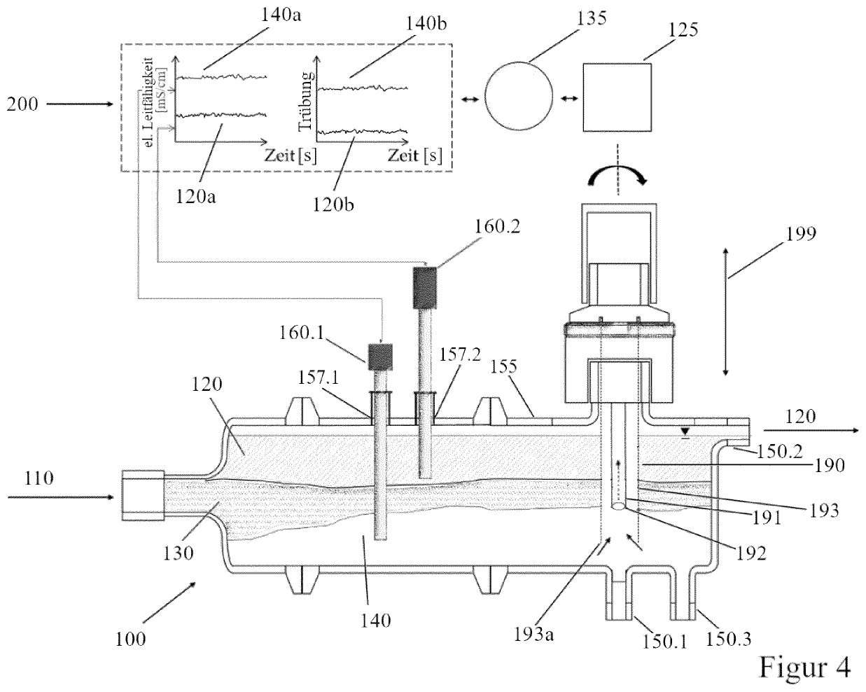

FIG. 4 shows a schematic side view in section of an exemplary embodiment of a separating device 100 according to the invention, which represents a continuous extraction with two probes (160.1 160.2) for measuring the electrical conductivity of the phases.

In the further exemplary embodiment of the invention shown in FIG. 4, the immersion tube 190 is shown in a different variant than in FIGS. 2 and 3. In this embodiment, the immersion tube 190 has an opening 192 at the end of the inner tube 191 and an overtube 193. Unlike in FIGS. 2 and 3, the immersion tube 190 is not connected to the lower outlet 150.1.

The overtube 193 has advantages because, in the example case shown, it allows only the heavy phase 140 to enter the opening 192. According to one embodiment, the overtube 193 can extend as far downward as possible, nearly to the bottom of the separating device 100, to prevent the light phase 120 and/or the intermediate phase 130 from entering the the intermediate space between the overtube 193 and the inner tube 191, even if the phase boundary interface(s) briefly decrease. The end 193a of the overtube 193 is located in the heavy phase 140. The position of the opening 192 then only plays a role for the removal rate.

The arrows in FIG. 4 symbolize how the heavy phase 140 first flows into the intermediate space between the inner tube 191 and the overtube 193 and is then sucked into the opening 192. The dotted arrow in the inner tube 191 of the immersion tube 190 indicates the direction of flow of the fluid in the inner tube 191, leading to an upper outlet (not shown).

In continuous operation, for example, the light phase 120 discharges at the upper outlet 150.2 and the heavy phase 140 is withdrawn through the immersion tube 190, for example, by a pump (not shown) or application of negative pressure. For equilibrium, the pump or vacuum is adjusted so that the sum of the outflow volume of the light phase 120 and the heavy phase 140 and, if necessary, the intermediate phase 130 is adjusted to be equal to the volume of the incoming two-phase system (ATPS) 110.

If fluctuations now occur and the phase boundary interface(n)/intermediate phase 130 drops, for example, then the pumping speed can be slightly reduced and the less heavy phase 140 is withdrawn until the phase boundary interface(n)/intermediate phase 130 rises, which can be determined by a value for the electrical conductivity of the probe 160.1 of the heavy phase 140 (=lower probe in FIG. 4), which is then again in the target value range. Then, for example, the original pumping speed/vacuum is again applied to the immersion tube 190.

If the phase boundary interface(s)/intermediate phase 130 increases, the pumping speed or vacuum can be increased and more heavy phase 140 is withdrawn. It decreases the phase boundary interface(s)/intermediate phase 130 again until the probe 160.2 for the light phase 120 indicates the electrical conductivity in the target value range again. The pumping speed or the vacuum at the immersion tube 190 can then be adjusted back to initial speed, for example.

The arrows in FIG. 4 symbolize how the heavy phase 140 first flows into the intermediate space and then is drawn out to the opening 192. The dotted arrow in the inner tube 191 of the immersion tube 190 indicates the direction of flow of the fluid in the inner tube 191, leading to an upper outlet (not shown).

The immersion tube 190 operates continuously in this case.

According to an alternative embodiment, the immersion tube 190 can also be used discontinuously to temporarily withdraw the light phase 120, intermediate phase 130 or heavy phase 140, depending on the state of the phase boundary interface(s). This does not require the presence of an overtube 193 in the immersion tube 190; however, this is possible.

For example, the light phase 120 flows out of the upper outlet 150.2 and the heavy phase 140 flows out of the separating device 100 continuously via the lower outlet 150.1. The continuous inflow of the aqueous two-phase system (ATPS) 110 into the separating device 100 can also play a role in the outflow behavior at the outlets 150.1 and 150.2. The opening 192 of the immersion tube 190 is then not immersed in any of the phases, for example.

If the phase boundary interface(s)/intermediate phase 130 increases (the conductivity probe 160.2 for the light phase 120 indicates a higher electrical conductivity), the opening 192 of the immersion tube 190 into the heavy phase 140 is adjusted (the immersion tube 190 is lowered) and the heavy phase 140 can be additionally pumped out/sucked away (for example, using a pump that generates a vacuum) until the value measured by the conductivity probe 160.2 in the light phase 120 is back within the target value range. The immersion tube 190 can then be returned to the initial position and the pump can be switched off.

If the phase boundary interface(s)/intermediate phase 130 drops (the conductivity probe 160.1 for the heavy phase 140 indicates a lower electrical conductivity than for the heavy phase 140), the immersion tube 190 can be moved into the light phase 120 (the immersion tube is driven up and repositioned) and the light phase 120 is pumped out (for example, sucked away with negative pressure generated by a pump) until the value measured by the conductivity probe 160.1 in the heavy phase 140 is again in the target value range. The immersion tube 190 can then return to its initial position and the pump can be switched off. Alternatively, the immersion tube 190 does not always have to return to the initial position after the interference has been eliminated. It is also sufficient here if the pump is switched off/the vacuum is removed.

Therefore, in the embodiment shown in FIG. 4, an overtube 193 that extends from the top to the bottom is used. In an embodiment analogous to FIG. 4, the overtube could also extend from the bottom to the top, wherein the immersion tube could serve as an outlet for the light phase 120. FIG. 4 would then be identical except for the overtube.

An overtube extending from the bottom to the top is advantageous, since only the light phase 120 would flow into the intermediate space between the inner tube 191 and the overtube, making it difficult for the intermediate phase 130 or the heavy phase 140 to enter the opening 192 in the inner tube 191.

According to this further embodiment (not shown), if the overtube were to extend from the bottom to the top and extend into the light phase 120 in order to remove the light phase 120 with the opening 192 in the inner tube 191, it would be expedient if the probe 160.2 were positioned lower in the light phase 120 than the upper end of the overtube. This would allow the electrical conductivity values to detect an excessive increase of the heavy phase 140 (lower phase) and/or, if applicable, the intermediate phase, before they could flow into the intermediate space between the inner tube 191 and the overtube and reach the opening 192 of the immersion tube.

An overtube extending from the bottom to the top (not shown) would therefore prevent the entry of the undesired phase (here: the intermediate phase 130 and/or the heavy phase 140), provided the phase boundary interface(s) do not increase too far.

Therefore, the above remarks on FIG. 4 apply analogously to this embodiment with the overtube extending from bottom to top.

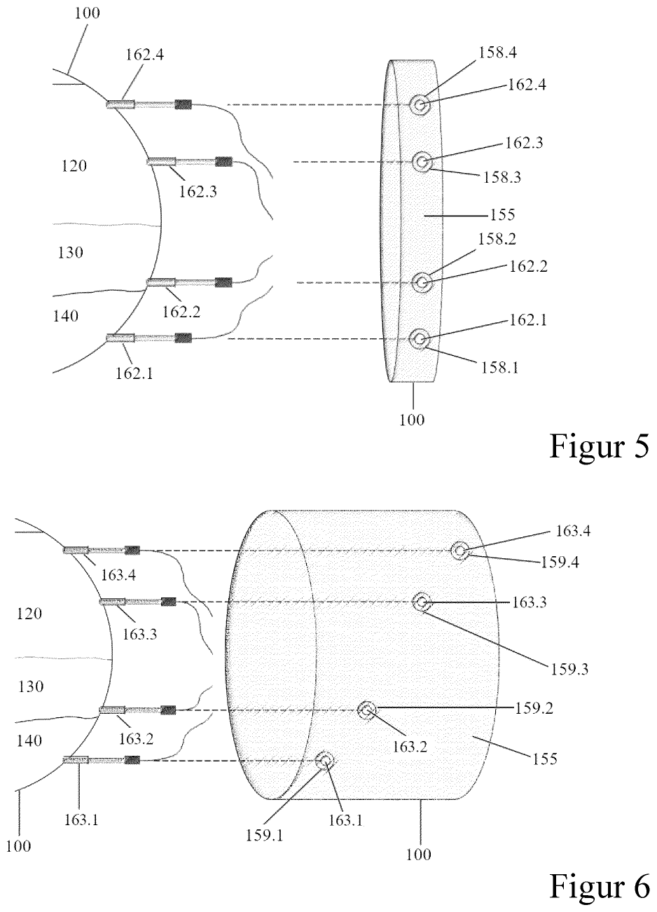

FIG. 5 shows a cross-section of an exemplary embodiment of a separating device 100 according to the invention with 4 insertion openings 158.1, 158.2, 158.3, 158.4, to each of which 4 probes 162.1, 162.2, 162.3, 162.4 are assigned, in a radial arrangement (left) and a side view thereof (right);

In the embodiment shown in FIG. 5, 4 probes 162.1, 162.2, 162.3 and 162.4 are shown in 4 insertion openings 158.1, 158.2, 158.3, 158.4 to be used in the separating device 100. On the left side of FIG. 5, a side view in section of a separating device 100 with a light phase 120, an intermediate phase 130 and a heavy phase 140 is shown, wherein the 4 insertion openings 158.1, 158.2, 158.3, 158.4, in each of which one of the probes 162.1, 162.2, 162.3 and 162.4 is located, are arranged in a radial arrangement in the outer wall 155 of the separating device 100. The probe 162.1 is provided for measuring the electrical conductivity of the heavy phase 140, the probe 162.2 for the intermediate phase 130, and the probes 162.3 and 162.4 are for the light phase. As can be seen from FIG. 5, the 4 insertion openings 158.1, 158.2, 158.3, 158.4, are arranged on an arc, which in the example shown corresponds to the shape of the outer wall 155 or the circumference of the separating device 100.

The right side of FIG. 5 shows a side view of the 4 insertion openings 158.1, 158.2, 158.3, 158.4 with the 4 probes 162.1, 162.2, 162.3 and 162.4 of the separating device 100. This arrangement is advantageous in terms of the more accurate determination and more reliable detection of the position of the individual phases based on the measured conductivity values and therefore enables the faster and more robust detection, control and monitoring of the method.

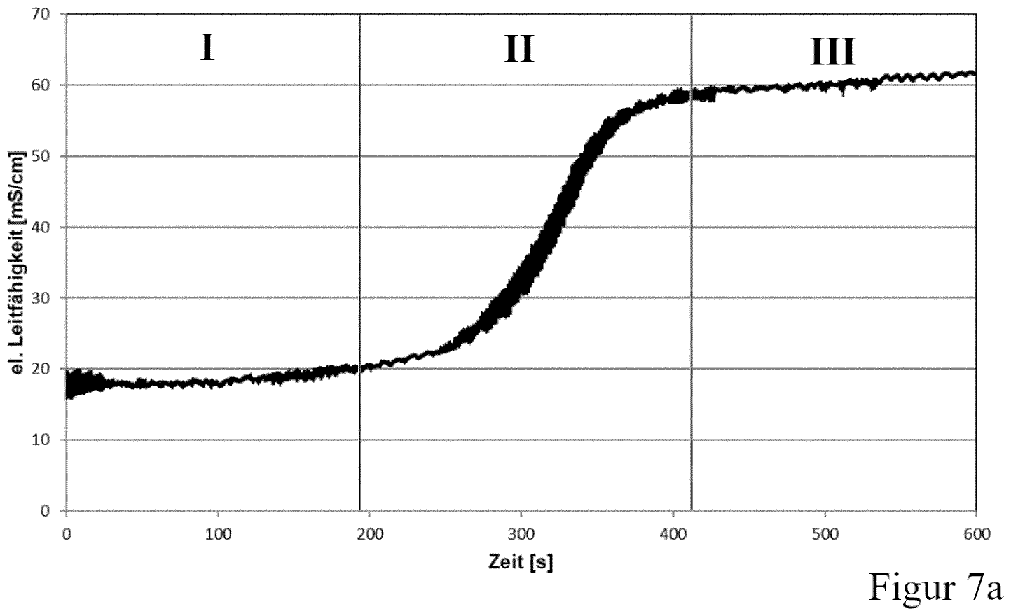

FIG. 6 also shows a cross-section of an exemplary embodiment of a separating device 100 according to the invention with the 4 probes 163.1, 163.2, 163.3 and 163.4 in 4 insertion openings 159.1, 159.2, 159.3, 159.4.

However, the 4 insertion openings 159.1, 159.2, 159.3, 159.4 are shown in a radial arrangement with horizontal distribution (left) and a side view thereof (right). A radially offset arrangement of each of the insertion openings 159.1, 159.2, 159.3, 159.4 also leads to more reliable detection of the individual phases and thus faster and more robust method control and monitoring.

In FIGS. 1 to 6, the one or more probes shown for measuring electrical conductivity may also be replaced by probes for measuring turbidity.

According to one embodiment, only probes for measuring electrical conductivity can be used in the separating device. According to a further embodiment, only probes for measuring turbidity can also be used in the separating device. According to another embodiment of the invention, a combination of probes for measuring electrical conductivity and probes for measuring turbidity can also be provided in the separating device.

In the following, the method of the invention is further illustrated by examples without limiting the invention herein.

### EXEMPLARY EMBODIMENTS

The method of the invention is explained in detail in the examples with reference to various embodiments.

### Preliminary Remarks

In the examples, a separator is filled with an aqueous two-phase system as the separating device, and the electrical conductivities or turbidities of the phases at the positions of the probes are recorded over time in each case. The tests are carried out with a separator filling volume of 920 ml and 470 ml. Aqueous two-phase phosphate, two-phase citrate and two-phase ammonium systems from the starting materials of polymer/salt/water are investigated. A polymer-rich light phase and a salt-rich heavy phase are formed. The phase-forming components or separate phases are preset in containers from which corresponding pumps deliver the components.

Before the fluids are added to the separator, the fluids pass through a mass flow meter and reach a static mixer. After passing the mixer, the fluid mixture runs into the separator, where the specific phases settle. The light phase can be removed at the top of an outlet. When an immersion tube is used, for example, it can remove the heavy phase through an opening in the form of an overflow valve. Two conductivity probes protrude into the separator to monitor the respective phase levels. Electrical conductivities are acquired and recorded with the aid a device manager for measured value detection and control. In the tests where cell culture was used as the starting material to be separated, a turbidity probe was also used, which was placed within the feed stream.

If an immersion tube is used in the examples, such immersion tube is used with an overtube.

### Continuous Extraction Tests

The method according to the invention was carried out with three different scales: two different laboratory scales with a DN35 separator and a DN50 separator and a pilot scale with a DN150 separator. “DN” is the nominal size and indicates the inner diameter of a tube and thus defines the size in device construction. Therefore, DN35 and DN50 represent laboratory scale and DN150 (80L scale) is already pilot scale, i.e., a larger scale that enables the production of larger product quantities.

Geometrically similar separators are used for each of the three scales investigated. Unless explicitly stated otherwise, static mixing elements (Kenics model) are used for phase mixing. For the different scales, correspondingly geometrically similar mixing sections are used, but adapted to the throughputs. The monitoring of the flows is carried out by means of mass flow meters. The measurement of electrical conductivity at suitable height positions is carried out by using two conductivity measuring probes. The main components and specifications of the test setup for carrying out the continuous extraction tests in the test facilities are listed in Table 2 below.

TABLE 2

| Number | Designation | | Comment |
|---|---|---|---|
| 1 | Gear pump (Ismatec) | — | Replaced by peristaltic |
| | | | pump in 80 L scale |
| 2 | Mass flow meter | — | Version M14, laboratory |
| | (Bronckhorst) | | scale |
| 3 | Mass flow meter | — | 80 L scale |
| | (Endress + Hauser) | | |
| 4 | Mixing section laboratory | — | Kenics-Helices |
| | (own production) | | |
| 5 | Mixing section Pilot | — | Kenics-Helices |
| | (own production) | | |
| 6 | Separator (DN35) | — | Smallest scale, no probe |
| | | | connection |
| 7 | Separator (DN50) | — | Typical laboratory scale, |
| | | | with probe connection |
| 8 | Separator (DN150) | — | Pilot scale, with probe |
| | | | connection and automatic |
| | | | filling level control |
| 9 | Conductivity probe | — | Part of the detection |
| | (Hamilton) | | system |
| 10 | Turbidity probe (Exxner) | — | Transmission, |
| | | | alpha = 880 nm |
| 11 | Labbox (Hitec Zang) | — | For measured value |
| | | | detection and control |

All process variables to be regulated and/or measured are detected and, if necessary, controlled by the device manager (Labbox). The implementation of the graphical user interface and the operation are performed by the software Labvision (HiTec Zang GmbH, Herzogenrath, Germany).

Laboratory scale DN35 and DN50

For this purpose, in Examples 5.1 to 5.3, the phase-forming components and a cell culture are initially conveyed into a static mixing section. Due to the mixing and the simultaneous presence of both aqueous phases, a mass transfer of target product and minor components takes place. Similarly, continuous mixing results in dispersion of bioparticles in one of the phases, creating an intermediate phase that binds bioparticles between its two phase boundary interfaces but no longer contains the target product.

After passing through the mixing section, the separator immediately follows, which is constructed as shown in FIG. 2. The entering mixture separates there along the flow direction into a light and a heavy phase. At the same time, a characteristic intermediate phase, which is a dispersion of the heavy phase stabilized by the bioparticles, is formed. By means of selective height adjustment of the immersion tube, which has an opening and therefore acts like an overflow valve, the heavy phase along with the intermediate phase is continuously removed from the separator. The light phase flows out of the upper outlet for the light phase from a critical filling level. All phases are collected in vessels.

Only for the sake of completeness, it is pointed out again that the “driven up” state of the immersion tube means that readjustment of the height of the immersion tube, namely upward, takes place. In the event that the immersion tube is fully driven up, the opening is, for example, above the fluid level in the separating device or the opening is driven up to such an extent that no more fluid can flow out of it. The immersion tube is then out of function. This is explained in detail in each of the examples.

The “driven down” state of the immersion tube means that a readjustment of the height of the immersion tube, namely downward, takes place. The immersion tube is in function during both driving up and driving down, unless otherwise specified.

The figures do not always show all the methods involved in driving up and driving down the immersion tube, as this would be too confusing and can be seen from the explanations.

### DN150 Pilot Scale

The test setup at the pilot scale, as described in Example 5.4, corresponds fundamentally to the information given above for the laboratory scale. The pilot-scale test setup differs from the laboratory-scale setup in terms of the pumps, mass flow meters, static mixing sections and size of the actual separator listed in Table 2.

### Susceptibility of the Method to Interference

Tests are also carried out on the susceptibility of the method to interference (see Example 4). The interference tests are carried out to determine to what extent the detection of the phase boundary interface(s) with the aid of the conductivity probes is suitable to draw conclusions about the adjusted system. For this purpose, varying systems were investigated upon start-up and in continuous operation after start-up with an aqueous two-phase phosphate system in the separators. In the tests, start-up is the period of time during which the phases settle in the separator and before and during which the phases segregate and are removed from the separator.

In Example 4.1, where the event of interference occurs in continuous operation, the separator was first filled and then continuous operation was adjusted with the aid of an immersion tube. Then, there was a switch to an interference system and the change in electrical conductivity was recorded.

In the start-up interference tests of Examples 4.2, 4.3, and 4.4, the interference system was placed in the separator from the beginning and continuous operation was stopped. Once this was reached, the system was switched to the correct system composition and the electrical conductivities were recorded.

Here, an interference system is a system that is either single-phase instead of two-phase, or has a composition that deviates from the usual composition by using a component of the aqueous two-phase system with 10% more or 10% less.

In the interference tests, the cell culture is replaced by deionized water.

The abbreviations used in the examples are summarized below:

List of abbreviations used

| Abbreviation | Meaning |
|---|---|
| ATPE | (Aqueous two-phase extraction) |
| ATPS | (Aqueous two-phase system) |
| COG | Cost of goods |
| DK | Dielectric constant |
| DN | Nominal diameter (diameter nominal) |
| DNA | Deoxyribonucleic acid |
| EMA | European Medicines Agency |
| FDA | Food and Drug Administration |
| HCP | Host cell protein |
| I | International Council for Harmonization of Technical |
| | Requirements for Pharmaceuticals for Human Use |
| LF | Conductivity |
| LP | Light phase |
| PEG | Polyethylene glycol |
| SP | Heavy phase |
| VE | Demineralized water |
| WCP | Wet cell paste |

### Example 1

### Illustration of the Start of a Continuous Extraction in a Separator in an Aqueous Two-Phase Phosphate System

Example 1 describes the start of a continuous extraction in a separator using an aqueous two-phase phosphate system. The start of extraction is also referred to as start-up; i.e., the phases settle but are not yet removed.

The aqueous two-phase phosphate system is constructed as follows:

TABLE 1
Mass fractions of aqueous
two-phase phosphate system

| | Proportion |
|---|---|
| Material | [wt. %] |
| Water | 44.5 |
| Buffer (40 wt. % | 40.0 |
| phosphate, pH 6.0) | |
| PEG400 solution | 15.5 |

In this test, the finished and prepared specific phases of the two-phase phosphate system are conveyed into the separator. Thereby, a total mass flow rate of 100 g/min was set, wherein the light phase is conveyed at 60 g/min and the heavy phase at 40 g/min in order to match the phase ratio of the system used. Therefore, the separately present phases were added together so that the two-phase system could be accurately characterized. Therefore, the test serves to illustrate the measurement of electrical conductivity. In addition, a preliminary test can be designed in this way in order to determine the expected electrical conductivities in advance.

With the aid of the conductivity probes, the electrical conductivities are recorded over time. The first probe (e.g., probe 160.1 in FIG. 2) is for measuring the electrical conductivity of the heavy phase and the second probe (e.g., probe 160.2 in FIG. 2) is for measuring the electrical conductivity of the light phase. To make the conductivity courses as comparable as possible, they are plotted so that the first probe detects the light phase at t=0. This makes it easier to compare the courses in different tests. The separator used here has a volume of 920 ml. The start-up behavior is recorded until light phase drips from the upper drain for the first time.

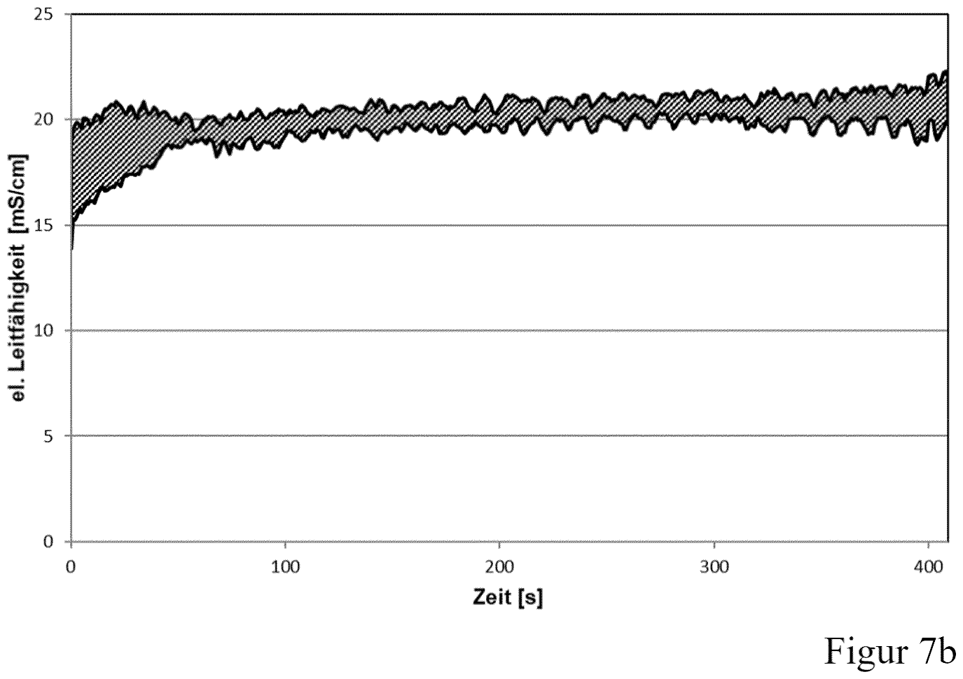

FIG. 7a shows the electrical conductivity measured values of the first probe during start-up, wherein the first probe (e.g., probe 160.1 in FIG. 2) is to detect the heavy phase. FIG. 7a shows an envelope curve comprising the upper and lower limits of the mean values with the respective standard deviations. The mean values and standard deviations represent at least duplicates.

In the curve of FIG. 7a, the start-up of the method is plotted against time using the measured electrical conductivity at the first probe and divided into sections I, II and III. In section I from approximately 0 to 200 s, the light phase is initially detected by the first probe at the start of the start-up. As the fluid of the two-phase system flowing into the separator increases, the probe then is increasingly immersed in the heavy phase in section II in the curve at approximately 200 to approximately 400 s; i.e., the electrical conductivity values increase steadily. In section III of the curve, only the heavy phase is then detected by the first probe.

FIG. 7b shows the electrical conductivity measured values from the second probe (e.g., probe 160.2 in FIG. 2) during start-up plotted against time in [s], wherein the second probe detects the light phase. FIG. 7b again shows an envelope curve comprising the upper and lower limits of the mean values with the respective standard deviations. The mean values and standard deviations represent at least duplicates. The curve in FIG. 7b shows the electrical conductivity of the second probe in the course of time. As soon as the upper light phase reaches the probe, a nearly constant electrical conductivity is measured, which shows that the light phase is nearly constant at the location of the probe measurement.

### Example 2

### Course of a Continuous Extraction in a Separator in an Aqueous Two-Phase Phosphate System

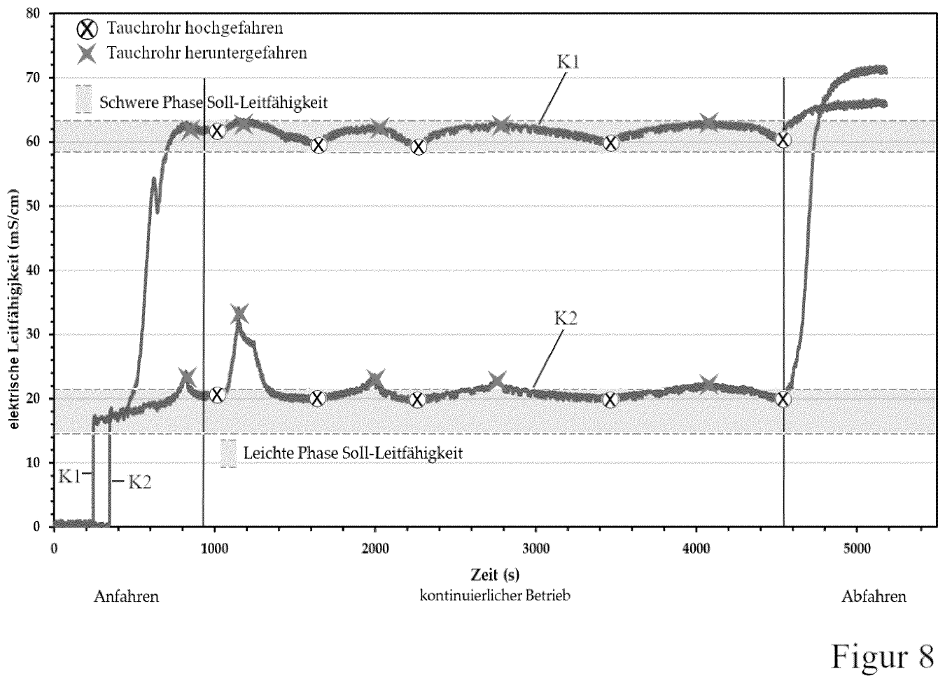

Example 2 describes a course of a continuous extraction in a separator using an aqueous two-phase phosphate system and explains the measured electrical conductivities of two probes, as shown for example in FIG. 2. FIG. 8 shows the measured values of the two probes in two curves K1 and K2. The first probe (e.g., probe 160.1 in FIG. 2) measures the electrical conductivity [mS/cm] of the heavy phase against time [s], shown in curve K1. The second probe (e.g., probe 160.2 in FIG. 2) measures the electrical conductivity [mS/cm] of the light phase against time [s], shown in curve K2.

The composition of the aqueous two-phase phosphate system is as described in Example 1.

As can be seen from FIG. 8, the method is divided into three sections, start-up from 0 to approximately 980 s, continuous operation from approximately 980 s to approximately 4580 s and shutdown from approximately 4580 s.

Continuous operation is shown only for an exemplary time period and is of course also possible for considerably longer periods; for example, continuous operation can be carried out for several hours or even longer.

In FIG. 8, the target conductivity range for the heavy phase (indicated as “target conductivity” in FIG. 8), at the top of FIG. 8, and the target conductivity range for the light phase (indicated as “target conductivity” in FIG. 8), at the bottom of FIG. 8, are each indicated as a range with dashed upper and lower limits. The corresponding phase is present in the target value ranges for the respective electrical conductivities. Leaving the target value ranges means that the probe no longer enters the corresponding phase. Therefore, if the conductivity target value range is left, readjustment is required. This is performed by the immersion tube 180 shown in FIG. 2 with an opening 182 and with an overtube 183. In Example 2, the immersion tube is used to remove heavy phase.

After the appropriate mass flows are set, the feed line is connected to the separator and the start-up of the method begins (0 to approximately 980 s). After 4 minutes, the first conductivity probe detects light phase for the first time (in curve K1, the first increase of electrical conductivity), which reaches the second probe after approximately 6 minutes (in curve K2, the first increase of electrical conductivity). At 7.5 minutes, the conductivity measured value of the first probe begins to rise to the maximum of approximately 62 mS/cm at 15 minutes (curve K1). After 9 minutes, light phase is drained for the first time at the top of the outlet. After 13.5 minutes, the immersion tube is used for the first time and is lowered to withdraw the accumulating heavy phase. In Example 2, the immersion tube therefore acts as a type of overflow valve that is used to selectively drain the heavy phase.

In FIG. 8, the downward movement of the immersion tube is represented by an asterisk, and the upward movement of the immersion tube is symbolized by an asterisk in a circle. More precisely, the opening of the inner tube is moved up and down here. The driving down and up of the immersion tube is indicated in both curves K1 and K2 at the same time in each case, since this can affect both curves to a different extent. Therefore, the immersion tube is not moved differently in the two curves K1 and K2, but an up or down movement of the immersion tube is indicated simultaneously in both curves K1 and K2.

The immersion tube was driven up after 18 minutes in order to determine the change in conductivity in both curves K1 and K2. That is, the immersion tube is driven up until it can no longer extract any fluid. It was found that the conductivity measured value of the second probe (curve K2) increases significantly, while the first probe (curve K1) hardly measures any change. This confirms that the first probe in continuous operation hangs directly in the heavy phase and reflects the conductivity of it.

### Example 3

### Course of a Continuous Extraction in a Separator in an Aqueous Two-Phase Citrate System

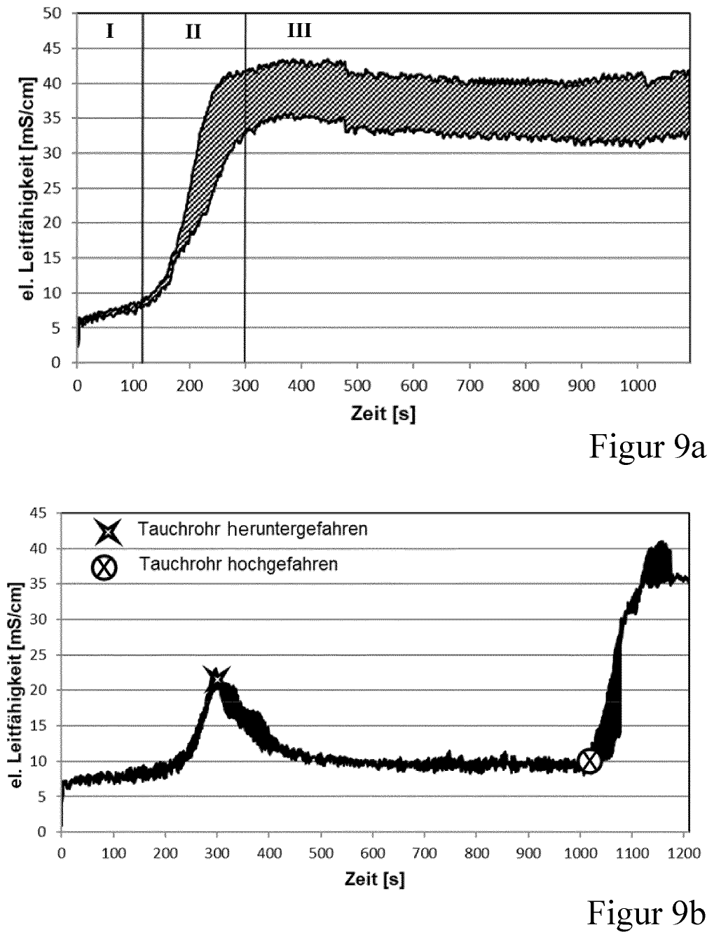

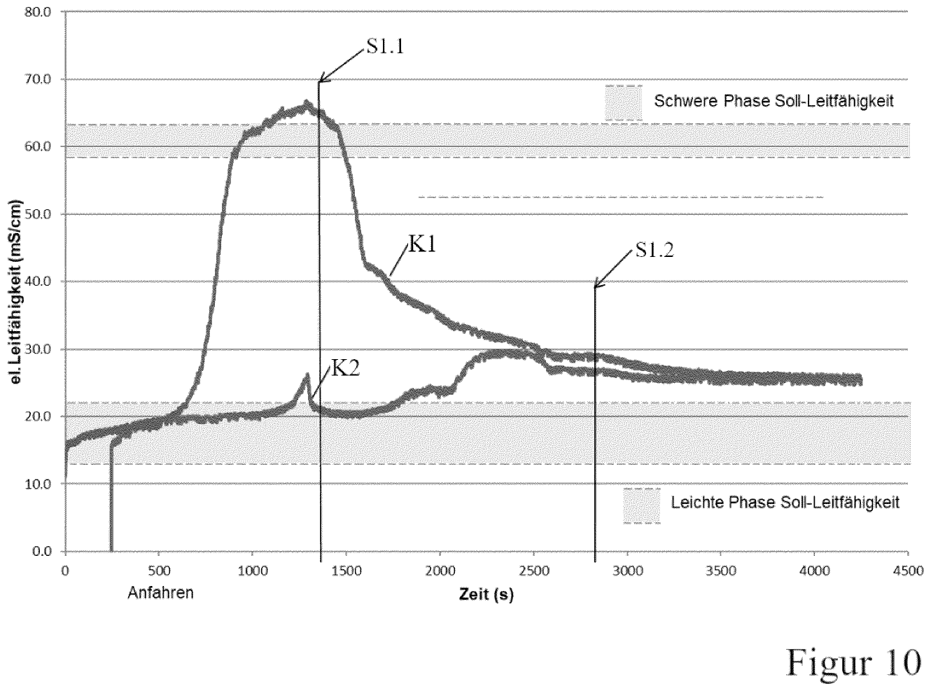

Example 3 describes a course of continuous extraction in a separator in an aqueous two-phase citrate system and shows the measured electrical conductivities of two probes in FIG. 9a and FIG. 9b.

The aqueous two-phase citrate system is constructed as follows:

TABLE 2
Mass fractions of aqueous
two-phase citrate system

| | Proportion |
|---|---|
| Material | [wt. %] |
| Water | 28.06 |
| Buffer (35 wt. % | 50.67 |
| citrate, pH 6.0) | |
| PEG400 solution | 21.27 |

In FIG. 9a, the electrical conductivities in [mS/cm] of the heavy phase are plotted against the time [s] measured by the first probe (e.g., probe 160.1 in FIG. 2). In FIG. 9b, the electrical conductivities in [mS/cm] of the light phase are plotted against the time [s] measured by the second probe (e.g., probe 160.2 in FIG. 2).

FIG. 9a shows the electrical conductivity measured values of the first probe, measured in the heavy phase, during start-up and also during continuous operation thereafter. FIG. 9a again shows an envelope curve comprising the upper and lower limits of the mean values with the respective standard deviations. The mean values and standard deviations represent at least duplicates.

The measurement curve in FIG. 9a, which shows the course of the recorded conductivity measured values of the first probe intended to detect the heavy phase, can again be divided into sections I, II and III. In section I from approximately 0 to approximately 110 s, the first probe initially detects the light phase. As the volume of the two-phase system filled in the separator increases, the probe then is increasingly immersed in the heavy phase in section II in the curve between approximately 110 s and approximately 300 s; i.e., the values for electrical conductivity increase steadily. In section III of the curve starting at approximately 300 s, the heavy phase is then detected by the first probe.

Therefore, as soon as the fluid level reaches the probe in FIG. 9a, the electrical conductivity value suddenly increases to 8 mS/cm. At such point in time, the electrical conductivity of the light phase is detected for the first time (section I in FIG. 9a). In the further course, the value increases first more weakly and then almost linearly to just below 35 mS/cm and then up to 45 mS/cm. At such point in time, pure heavy phase is detected for the first time (section II in FIG. 9a). Such value for electrical conductivity remains almost constant over the further duration of the test, since the first probe always detects the heavy phase and this does not decrease during the tests (section III in FIG. 9a). To ensure that the heavy phase does not increase or decrease too far, an immersion tube used with an opening is inserted and readjusted as necessary as a function of the electrical conductivities recorded.

If the target product is located in the light phase, it is expedient if at the end of continuous operation, during the so-called shutdown, the immersion tube is driven up again (the immersion tube stops removing heavy phase), so that heavy phase is enriched and thereby displaces as much light phase and thus product from the separator as possible. This is also shown, for example, in FIG. 8 at approximately 4580 s, where the immersion tube is driven up. Such procedure of increasing the yield of the light phase by displacing the light phase with the additional heavy phase is described in detail in FIG. 3 above.

The driving up and driving down of the immersion tube can be seen particularly clearly in FIG. 9b. FIG. 9b shows the course of the electrical conductivity measured values of the second probe (e.g., probe 160.2 in FIG. 2), which is to detect the light phase, during start-up and subsequent continuous operation, plotted against time in [s]. FIG. 9b again shows an envelope curve comprising the upper and lower limits of the mean values with the respective standard deviations. The mean values and standard deviations represent at least duplicates.

Thus, the curve in FIG. 9b shows the measured electrical conductivity values of the second probe in the course of time. As soon as the second conductivity probe is immersed in the fluid, the conductivity measured value jumps to approximately 8 mS/cm. At first the measured value increases slowly; as soon as a conductivity of 10 mS/cm is reached, the measured value increases almost linearly to a maximum value. The significant increase can be explained by the fact that as soon as light phase exited at the upper outlet, the immersion tube was not driven down directly. This causes the heavy phase to accumulate, which the second probe indicates by increasing the conductivity measured value.

At such point in time, i.e., at approximately 300 s, the immersion tube is then driven down (indicated by the asterisk in the curve). At such point in time, the electrical conductivity has risen to slightly above 20 mS/cm and has therefore left the target value range for electrical conductivity values of the light phase of aqueous two-phase citrate systems in the range of 5-15 mS/cm. In other words, the heavy phase is approaching the second probe.

Therefore, the immersion tube is driven down (asterisk in the curve in FIG. 9b) and from such point in time on, the heavy phase flows out through the immersion tube, causing the heavy phase in the separator to decrease. At the same time, the light phase also decreases, such that the second probe is again in pure light phase. This is shown in FIG. 9b by the decrease of electrical conductivity values to approximately 10 mS/cm. Once the upper light phase reaches the second probe again, a nearly constant electrical conductivity is measured, which shows that the light phase is nearly constant at the location of the probe measurement. Therefore, at a measured value of approximately 10 mS/cm, the second probe here detects pure, light phase.

The immersion tube in the embodiment with an overtube, the opening of which is in the inner tube, can be operated like an overflow valve, as described for example in FIGS. 2 and 3. Thereby, the fluid is allowed to flow out by gravity. Alternatively, the immersion tube with its opening can also suck away the heavy phase, as explained in FIG. 4. This does not change the actual procedure.

After holding steady state at approximately 10 mS/cm for a relatively short time, as shown in FIG. 9b, in orderly to solely illustrate continuous operation, the shutdown begins. Of course, continuous operation could be maintained much longer, for example, several hours or even longer. However, this would not change the available information and its significance.

For shutdown, the immersion tube is driven up after approximately 1000 s (indicated by the asterisk in the circle in FIG. 9b). This means that the immersion tube is no longer in operation, such that the heavy phase accumulates again. This causes the conductivity measured value to rise again until it reaches the value of the heavy phase. This also shows the large differences in the electrical conductivities of the heavy phase (citrate system, heavy phase: 30-50 mS/cm, see Table 1) compared to the light phase (citrate system, light phase: 5-15 mS/cm, see Table 1). As soon as the second measuring probe indicates the conductivity measured value of the heavy phase, the test is terminated, since a further increase of the heavy phase can no longer be detected.

### Example 4

### Investigation of the Susceptibility of the Method to Interference

The investigation of the susceptibility of the method to failures is intended to determine how reliably the method works and to what extent conclusions can be drawn from the phase detection about the system adjusted in the separator in continuous operation.

### Example 4.1

In Example 4.1, a single-phase system is added to the separator instead of a two-phase system. Such a single-phase system is also called an interference system.

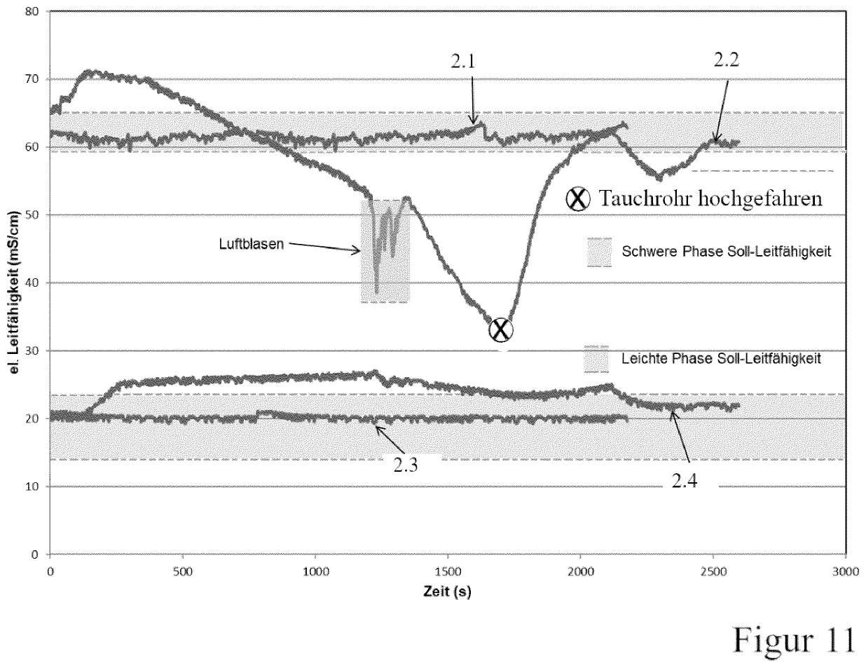

An aqueous two-phase phosphate system is used as the initial system. The interference system is produced by introducing 50% less phosphate buffer. This makes the interference system single-phase. FIG. 10 shows the conductivity measured values from Example 4.1. Curve K1 gives the electrical conductivity values of the first probe, which measures the heavy phase. Curve K2 gives the electrical conductivity values of the second probe, which measures the light phase.

Initially, start-up with a two-phase phosphate system is carried out in the usual manner, as described in Examples 1 and 2. After the start-up with the phosphate system is completed, the interference system is introduced into the separator at (S1.1). As soon as the single-phase interference system is conveyed into the separator, the conductivity of the probe for the heavy phase (curve K1) drops. The drop in conductivity to a value of 60 mS/cm can be explained by the adjusting of the immersion tube (not shown), which here again acts as an overflow valve. The immersion tube was subsequently not adjusted in this test and was held at a constant setting used in normal two-phase phosphate system. The conductivity value then drops almost linearly to 40 mS/cm and then decreases at a slower rate to 25 mS/cm. Re-introducing the interference system at (S1.2) into the separator no longer leads to major changes. The conductivity of the second probe, shown in curve K2, slowly increases from 20 mS/cm to 30 mS/cm.

### Example 4.2

In Example 4.2, the water content of each of the heavy and light phases is varied. An aqueous two-phase phosphate system is used in continuous operation.

The recorded values for conductivities are plotted against time in [s] in FIG. 11. The electrical conductivities of the heavy phase are measured with the first probe and the light phase with the second probe. Two tests were carried out: In the first test with a two-phase phosphate system containing a water content of −10 wt. % compared to a two-phase phosphate system containing the correct water quantity, and in the second test with a two-phase phosphate system containing a water content of +10 wt. % compared to a two-phase phosphate system containing the correct water quantity. Table 3 below shows the quantities used.

TABLE 3
Aqueous two-phase phosphate system
with varying initial water weight

| | −10% | +10% | Two-phase phosphate |
|---|---|---|---|
| | water | water | system with correct |
| Material | [wt. %] | [wt. %] | water quantity [wt. %] |
| Water | 41.9 | 46.9 | 44.5 |
| Buffer (40 wt. % | 41.9 | 38.3 | 40.0 |
| phosphate, pH 6.0) | | | |
| PEG400 solution | 16.2 | 14.8 | 15.5 |

Both tests are shown in FIG. 11. The labeling of FIG. 11 is as follows:
Curve 2.1 . . . heavy phase with −10 wt. % water;
Curve 2.2 . . . heavy phase with +10 wt. % water;
Curve 2.3 . . . light phase with −10 wt. % water;
Curve 2.4 . . . light phase with +10 wt. % water.

The arrow in FIG. 11 indicates air bubbles in the system that cause measurement errors.

The phrase “heavy phase with −10 wt. % water” means that the electrical conductivity for the heavy phase is shown in the curve in FIG. 11, wherein the two-phase phosphate system used has a water content 10 wt. % lower than a conventional two-phase phosphate system with the correct water content.

The phrase “heavy phase with +10 wt. % water” means that the electrical conductivity for the heavy phase is shown in the curve in FIG. 11, wherein the two-phase phosphate system used has a water content 10 wt. % higher than a conventional two-phase phosphate system with the correct water content.

### Example 4.3

In Example 4.3, the polymer content of the polyethylene glycol (PEG) of heavy and light phases is varied, respectively. An aqueous two-phase phosphate system is used. Work is carried out in continuous operation.

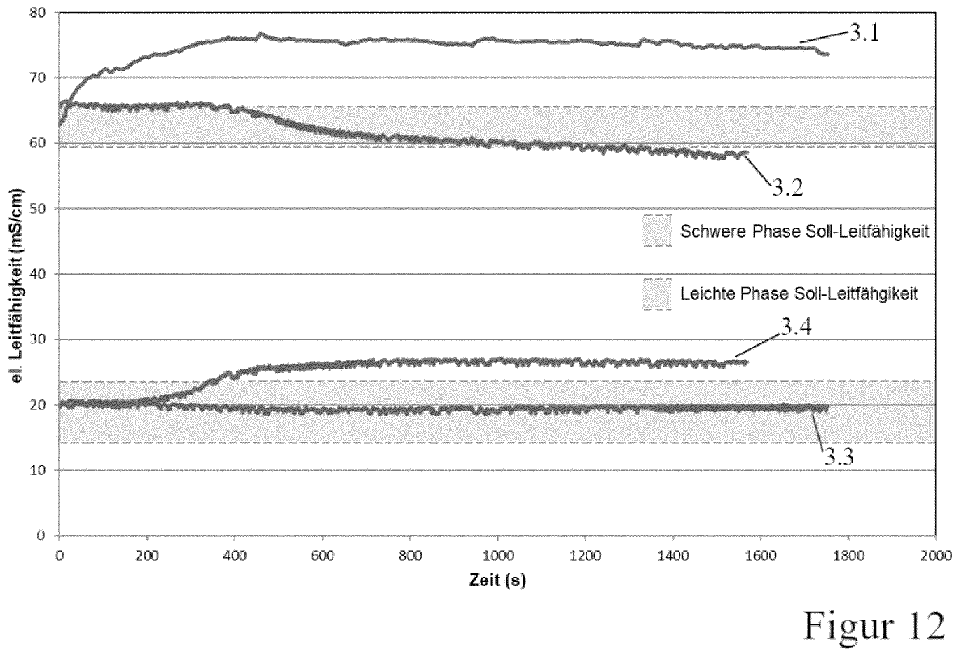

The recorded values for conductivities are plotted against time in [s] in FIG. 12. The electrical conductivities of the heavy phase are measured with the first probe and the light phase with the second probe. Two tests were carried out: In the first test with a two-phase phosphate system containing a polymer content of −10 wt. % compared to a two-phase phosphate system containing the correct polymer quantity, and in the second test with a two-phase phosphate system containing a polymer content of +10 wt. % compared to a two-phase phosphate system containing the correct polymer quantity.

Table 4 below shows the quantities used.

TABLE 4
Aqueous two-phase phosphate system
with varying initial polymer weight

| | −10% | +10% | Two-phase phosphate |
|---|---|---|---|
| | PEG | PEG | system with correct |
| Material | [wt. %] | [wt.%] | polymer quantity [wt. %] |
| Water | 45.0 | 43.8 | 44.5 |
| Buffer (40 wt. % | 40.9 | 39.4 | 40.0 |
| phosphate, pH 6.0) | | | |
| PEG400 solution | 14.1 | 16.8 | 15.5 |

Both tests are shown in FIG. 12. The labeling of FIG. 12 is as follows:
Curve 3.1 . . . heavy phase with +10 wt. % PEG;
Curve 3.2 . . . heavy phase with −10 wt. % PEG;
Curve 3.3 . . . light phase with +10 wt. % PEG;
Curve 3.4 . . . light phase with −10 wt. % PEG;

### Example 4.4

In Example 4.4, the buffer salt is varied in the form of the phosphate salt of an aqueous two-phase phosphate system in each of the heavy and light phases. Work is carried out in continuous operation.

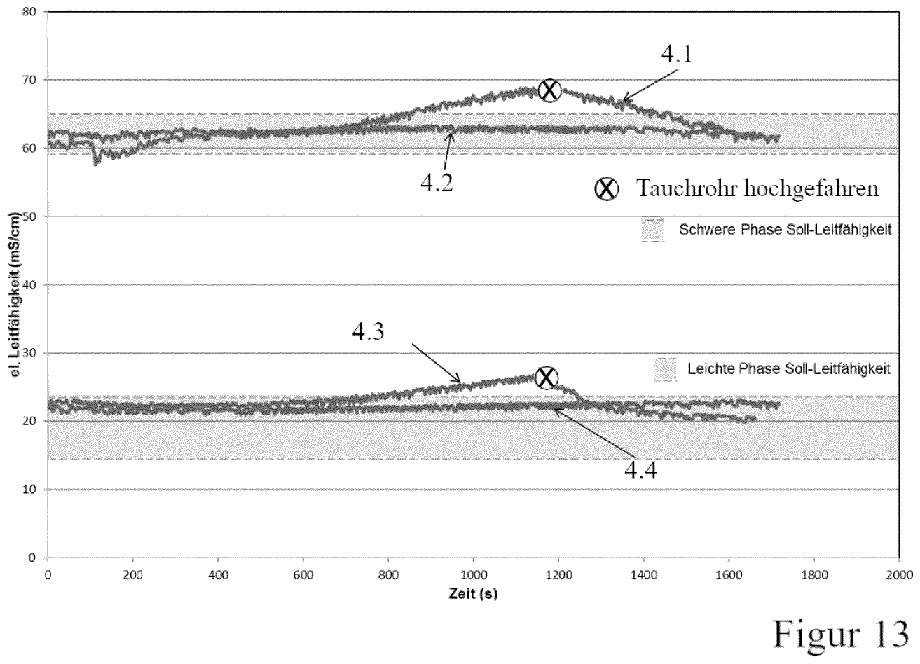

The recorded values for conductivities are plotted against time in [s] in FIG. 13. The electrical conductivities of the heavy phase are measured with the first probe and the light phase with the second probe. Two tests were carried out: In the first test with a two-phase phosphate system containing a buffer salt content of −10 wt. % compared to a two-phase phosphate system with correct buffer salt quantity, and in the second test with a two-phase phosphate system containing a buffer salt content of +10 wt. % compared to a two-phase phosphate system with correct buffer quantity.

In Example 4.4., the separator is filled directly with the interference composition. Table 5 below shows the quantities used.

TABLE 5
Aqueous two-phase phosphate system with varying initial
buffer salt weight

| | −10% | +10% | Two-phase phosphate |
|---|---|---|---|
| | buffer | buffer | system with correct |
| Material | [wt. %] | [wt. %] | buffer salt quantity [wt. %] |
| Water | 46.3 | 42.8 | 44.5 |
| Buffer (40 wt. % | 37.5 | 42.3 | 40 |
| phosphate, pH 6.0) | | | |
| PEG400 solution | 16.2 | 14.9 | 15.5 |

Both tests are shown in FIG. 13. The labeling of FIG. 13 is as follows:
Curve 4.1 . . . heavy phase with +10 wt. % phosphate buffer;
Curve 4.2 . . . heavy phase with −10 wt. % phosphate buffer;
Curve 4.3 . . . light phase with +10 wt. % phosphate buffer;
Curve 4.4 . . . light phase with −10 wt. % phosphate buffer.

Based on all the tests in Examples 4.1 to 4.4 and the four different aqueous systems (variation of the phase number and the content of water, buffer and polymer), shown in FIGS. 10, 11, 12 and 13, even slight deviations from the target state can be detected with the measuring principle based on the conductivity profile. Even over longer process times (>3 hours), the measurement is reliable (as will also be demonstrated below) and thus enables optimum efficiency in the separation of light and heavy phases, maximizing the yield of the process.

### Example 5

Continuous Extraction in the Separator with a Cell Culture for Purification and Enrichment of Antibodies

In the following, several examples will show that the electrical conductivities of the heavy and light phases change in the presence of bioparticles, and that the intermediate phase and heavy phase formed in the method can also have electrical conductivity values that differ from the expected conductivities. The intermediate phase represents a mixture of heavy phase in dispersed form together with cells. Both the intermediate phase and the heavy phase may have, in some cases, significantly reduced electrical conductivity than would be expected, which is attributed to the presence of cells and other conductivity-lowering bioparticles.

Example 5 shows how the detection of electrical conductivity is used to separate the target product in the target phase (in this example, the light phase) from the counterphase (in this example, the heavy phase) and the intermediate phase with cells.

### Example 5.1

### DN50 Scale—Citrate System

Example 5.1 describes a continuous extraction according to the measuring principle of the method according to the invention in a separator with a cell culture. The test serves as an orientation and illustration of the measuring principle and was carried out on a laboratory scale. A two-phase citrate system was used to purify and enrich the target product, in this case an antibody, from a cell culture. The initial weight of phase-forming components and cell culture along with the adjusted mass fractions are summarized in Table 6:

TABLE 6
Initial weight and mass fractions for Example 5.1

| | Initial weight | Actual mass | Target mass |
|---|---|---|---|
| Material | [g] | fraction [wt. %] | fraction [wt. %] |
| Cell culture | 949.78 | 28.0 | 28.2 |
| Citrate buffer | 1696.71 | 50.2 | 50.5 |
| (35 wt. %, pH 6.0) | | | |
| PEG 400 | 736.51 | 21.8 | 21.3 |

The aqueous two-phase citrate system, constructed as indicated in Table 6, has already been suitably mixed, such that the cell culture components have partitioned between the phases. The target product accumulates in the light phase in Example 5.1. The mixture is then conveyed via a pump into a separator at a mass flow rate of 28 g/min. With such conveying rate, it takes approximately 17 minutes for the light phase, comprising the target product in the form of antibodies, to exit at the top of the outlet. The test overview for Example 5.1 is given in Table 7 below.

TABLE 7
Test overview for Example 5.1

| | | |
|---|---|---|
| Mass flow rate | 28 | g/min |
| Total duration | 120 | min |
| Filling time | 17 | min |

| | |
|---|---|
| LPₜₒₜ | 1420 ml ± 71 ml |
| SPₜₒₜ | 820 ml ± 41 ml |
| Antibody content | 4.6 g/l ± 0.1 g/l (cell culture) |

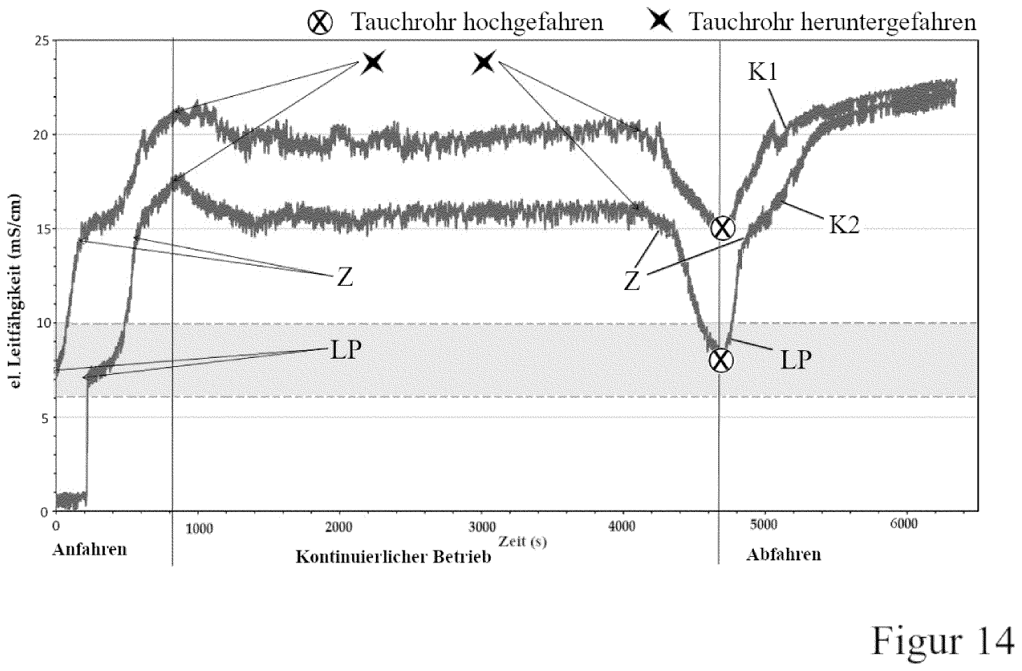

The recorded electrical conductivities of Example 5.1 using two probes, one probe for the heavy phase (e.g., probe 160.1 in FIG. 2) and one probe for the light phase (e.g., probe 160.2 in FIG. 2), are plotted against time in [s] in FIG. 14. The method can again be divided into three sections: start-up from 0 to approximately 810 s, continuous operation from approximately 810 s to 4680 s and shutdown from approximately 4680 s onward.

Upon starting up, the typical course is recognizable. The first deeper probe, which is to detect the heavy phase, shows light phase first with a value for electrical conductivity of approximately 8 mS/cm and then increases almost constantly up to 15 mS/cm (curve K1). At 15 mS/cm, a kink in the slope can be seen. Here, the intermediate phase with the cells is detected. The intermediate phase is designated Z in FIG. 14 and the arrows indicate its detection. The second probe shows a similar course upon start-up (curve K2). Electrical conductivity suddenly jumps to 8 mS/cm. Light phase is detected here for the first time. Initially, electrical conductivity does not increase as much until it changes to an almost linear increase. At 15 mS/cm, it can be seen that the values for electrical conductivity increase less sharply; here, the intermediate phase Z is detected with the cells. As soon as the second probe reaches a conductivity value of 17 mS/cm, the immersion tube is driven down (asterisk in FIG. 14) and a continuous process is set. Thereby, a difference in electrical conductivities between the phases of 5 mS/cm can be seen.

Contrary to expectations, the heavy phase has a much lower conductivity than expected. This is likely due to the presence of cells and other conductivity-lowering bioparticles.

At the end of continuous operation, a drop in electrical conductivities can be seen. This is due to the fact that the immersion tube is adjusted to be deeper here (readjustment of the immersion tube), by which more heavy phase is withdrawn due to the higher hydrostatic pressure. This was to test the response of the system and detect the electrical conductivities. The values for electrical conductivity decrease directly as the immersion tube is lowered (asterisk in FIG. 14): Thereby, the measured value of the second probe (curve K2) drops down to that of the continuous light phase, designated LP in FIG. 14. The detected electrical conductivity of the first probe decreases to the same extent; however, the difference is not as large as for the second probe. The lowest value for electrical conductivity is 15 mS/cm; here, just the intermediate phase Z with the cells is detected. The immersion tube is then driven up completely (asterisk in the circle in FIG. 14) in order to prevent the intermediate phase from being detected due to a further decrease of the heavy phase. Therefore, the shutdown begins with the immersion tube fully ramping up at approximately 4680 s (asterisk in the circle in FIG. 14), causing the electrical conductivity measured values to increase rapidly. At approximately 15 mS/cm, a kink is again visible in the second probe (curve K2). Here as well, the intermediate phase Z is detected with cells; this is indicated in FIG. 14 with arrows to Z (Z means intermediate phase). Contrary to expectations, the values for the electrical conductivity of the intermediate phase with cells are not in the range of 30-40 mS/cm. This is attributed to the presence of cells and other conductivity-lowering bioparticles. The values for electrical conductivity then rise to over 20 mS/cm. The electrical conductivities of the two probes eventually converge rapidly to the same value.

As soon as the immersion tube has been driven down at approximately 4200 s (see asterisk in FIG. 14), the conductivity measured value of the second probe (curve K2) immediately drops to the electrical conductivity of the pure light phase (5 to 15 mS/cm). Thus, the electrical conductivity can be used to locate the intermediate phase in such system. Although the first probe did not detect a pure heavy phase in continuous operation, the electrical conductivity of which should be in the range of 30 to 50 mS/cm, it is still possible to determine which electrical conductivity measured value belongs to the intermediate phase with the aid of the electrical conductivity values from the start-up. The conductivity measured value of the intermediate phase Z can be detected in the first plateau after detection of the light phase LP. In the intermediate phase, which here changes into the heavy phase, a conductivity gradient is present, which means that the start of the intermediate phase can be well estimated with the aid of the detected electrical conductivity value.

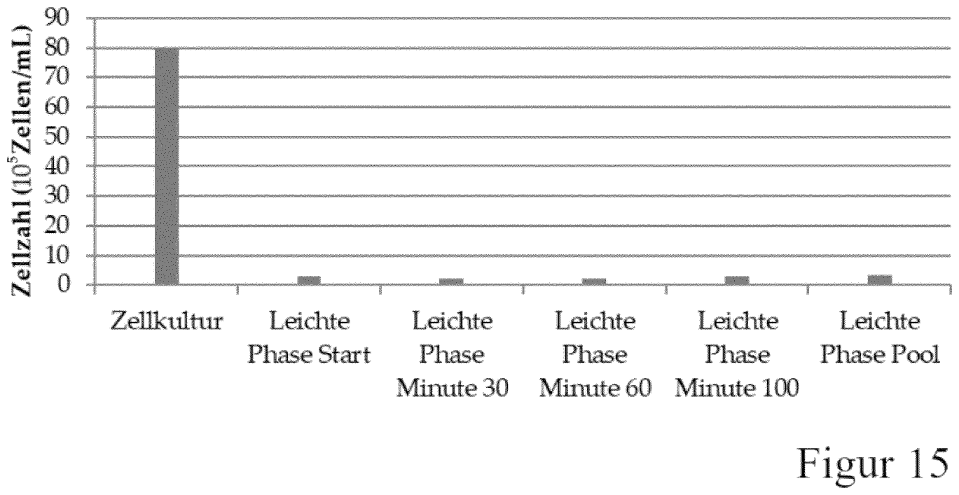

FIG. 15 shows the cell number in [10⁵ cells/mL] of the cell culture used in Example 5.1 and the cell number of the light phase obtained during the method (curve K2), which contains the target product. It can be seen that at all times there are consistently fewer cells in the light phase obtained than in the cell culture. The reduced cell number can also be observed in the light phase obtained after extraction (light phase pool in FIG. 15).

Thus, using Example 5.1, it can be shown that the product phase (light phase) can be uniquely identified with the aid of the measurement of electrical conductivities and constantly separated from the other phases via continuous extraction.

The antibodies are enriched in the light phase separated in Example 5.1. and are present in purified form. The cells could be depleted by 1.4 log steps.

### Example 5.2

### DN50 Scale—Aqueous Two-Phase Phosphate System

### Reference Example

Example 5.2 describes a continuous extraction according to the measuring principle of the present method in a separator using a cell culture. The test is only for orientation and illustration of the measuring principle and was carried out on a laboratory scale. A two-phase phosphate system was used to purify and enrich the target product, in this case antibodies, from a cell culture. The initial weight of phase-forming components and cell culture along with the adjusted mass fractions are summarized in Table 8:

TABLE 8
Initial weight and mass fractions

| Material | Initial weight [g] | Mass fraction [wt. %] |
|---|---|---|
| Cell culture | 970 | 45.5 |
| Phosphate buffer | 833.03 | 39.0 |
| (40 wt. %, pH 6.0) | | |
| PEG 400 | 330.29 | 15.5 |

The aqueous two-phase phosphate system, constructed as indicated in Table 8, has already been suitably mixed in a static mixer, such that the cell culture components partition between the phases. The target product, here: antibody, accumulates in the light phase in Example 5.2. The mixture is then conveyed via a pump into a separator at a total mass flow rate of 50.7 g/min. The reactants are placed in beakers and conveyed by gear pumps into the static mixers and then into the separator. The light phase and the heavy phase are absorbed.

A test overview for Example 5.2 is given in Table 9 below.

TABLE 9
Test overview for Example 5.2

| | | |
|---|---|---|
| Mass flow rate | 50.7 | g/min |
| Total duration | 58 | min |
| Filling time | 10 | min |

| | |
|---|---|
| LPₜₒₜ | 1010 ml ± 50.5 ml |
| SPₜₒₜ | 700 ml ± 35 ml |
| Antibody content | 3.1 g/l ± 0.4 g/l (cell culture) |

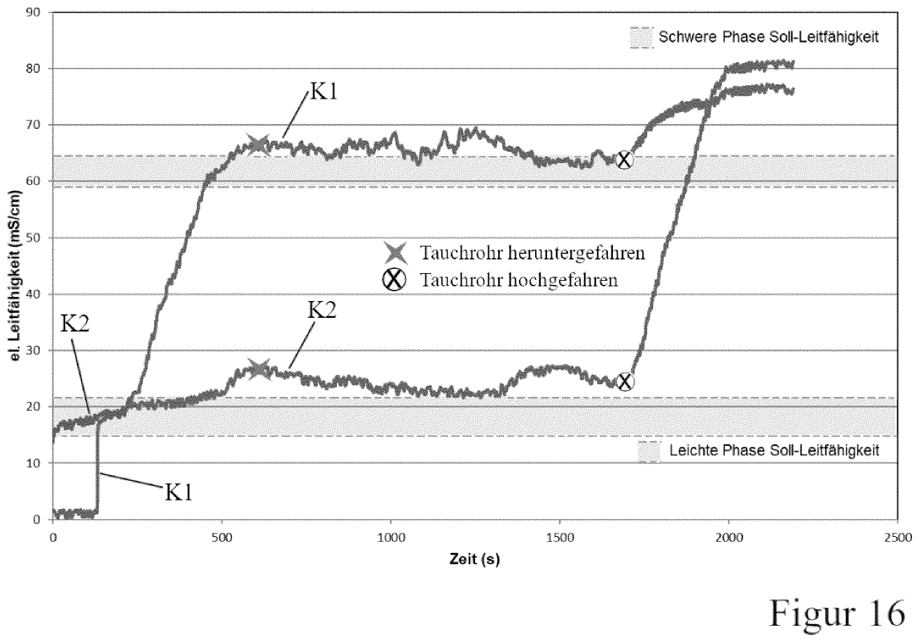

The recorded electrical conductivities of Example 5.2 using two probes, one probe for the heavy phase (e.g., probe 160.1 in FIG. 2) and one probe for the light phase (e.g., probe 160.1 in FIG. 2), are plotted against time in [s] in FIG. 16.

In FIG. 16, curve K1 shows the measured values for the electrical conductivity of the first probe, which is to detect the heavy phase, and curve K2 shows the measured values for the electrical conductivity of the second probe, which is to detect the light phase. The measured values of electrical conductivity of the first probe in curve K1 increases rapidly from 0 mS/cm to 16 mS/cm during start-up. The conductivity curves of the two probes intersect at 19 mS/cm. Thereafter, the measured value for the electrical conductivity of the first probe in curve K1 increases almost linearly to a value of 65 mS/cm. Here, the curve flattens out. The immersion tube is driven down (asterisk in curve K1 in FIG. 16), then continuously removes heavy phase and a continuous method is thus established. The conductivity measured value of the first probe varies between 63.2 mS/cm and 69.3 mS/cm for the heavy phase. This represents the continuous operation of the method.

To shut down continuous extraction, the immersion tube is driven up (asterisk in the circle in curve K1 in FIG. 16) and thus stops removing heavy phase. According to this, the electrical conductivity values of the first probe asymptotically approach a value of 77 mS/cm.

In curve K2, the electrical conductivities measured by the second probe for the light phase, the probe detects a value of 17 mS/cm at the beginning. This then increases to 20 mS/cm. Such value is detected constantly for a short time and increases in the further course to 25 mS/cm. At such point in time, the immersion tube is lowered (asterisk in curve K2 in FIG. 16) and the immersion tube begins to remove the heavy phase. This can be performed by gravity, wherein the opening present in the immersion tube, i.e., in the inner tube, is brought into a position, such that the heavy phase flows out. The immersion tube then works like an overflow valve. According to another embodiment of the immersion tube, the heavy phase can also be sucked away through the immersion tube. Both alternatives are equally suitable and can be used to remove the heavy phase.

The use of the immersion tube can be seen from the decrease of electrical conductivities in curve K2. The measured electrical conductivities by the second probe decrease almost steadily throughout continuous operation until a value of 22 mS/cm is reached at the end. The electrical conductivity then rises to just under 25 mS/cm.

Subsequently, the immersion tube is driven up (asterisk in the circle in curve K2 in FIG. 16) and therefore stops removing heavy phase; the measured conductivities of the second probe then increase almost linearly (curve K2) until the measured value of the first probe is reached (curve K1).

An analysis of the heavy and light phases shows that the partitioning of target product into the light phase and impurities, in particular cells, into the heavy phase and/or intermediate phase has not occurred. It can also be seen from the clear turbidity of the light phase that cell harvesting has not worked in such system and the cells remain in the light phase. The light phase is a disperse phase and should actually be clearer. In contrast, the heavy phase settles faster than would be expected and is also very clear. An intermediate phase with cells is recognizable between the two phases. The cells remain in the light phase and may not be withdrawn from the immersion tube, since it is designed to remove the heavy phase. There are also distinct streaks of cells visible in the inlet of the separator. Thus, a phase inversion has occurred in the system. This is attributed to insufficient mixing. Therefore, in the method according to the invention, mixing of the aqueous two-phase system is carried out while maintaining a mass transfer, such that the target product has enriched and is substantially present in only one of the phases, namely the expected phase, and the impurities have enriched and are substantially present in the counterphase.

The electrical conductivities of the heavy phase in Example 5.1. are in the range of the pure heavy phase (without cells) as obtained in Example 4.2 (FIG. 11) in one of the tests when investigating the susceptibility of the method to interference and varying the water content. Such conductivity is comparable to the conductivity of a cell-free phase with product. Thus, the formation of an intermediate phase of non-coalesced heavy phase and cells has not occurred here. Such undesirable effect serves as a negative example here.

It should be explicitly pointed out in this connection that Example 5.2. does not represent an example according to the invention, since, here, mixing under mass transfer, as indicated in the teaching of the method according to the invention, was not carried out.

It is noteworthy that the measuring principle according to the method of the invention nevertheless works; i.e., the heavy phase and the light phase could be identified and separated by their electrical conductivities. Thereby, the fault lies in the lack of mixing that can be readily avoided.

### Example 5.3

### DN50 Scale—Aqueous Two-Phase Phosphate System

To demonstrate that sufficient mixing in Example 5.2 eliminates the problems encountered there, Example 5.2 was repeated and the two phases were again transferred to the separator. However, the volume flow is reduced from 50.7 g/min to 25 g/min. This is to extend the dwell time in the static mixers to produce higher mixing. 810 ml light phase and 310 ml heavy phase are conveyed into the separator. For the initial weight of phase-forming components and cell culture along with the adjusted mass fractions, please refer to Table 8. A test overview for Example 5.3 is given in Table 10 below.

TABLE 10
Test overview for Example 5.3

| | | |
|---|---|---|
| Mass flow rate | 25 | g/min |
| Total duration | 52 | min |
| Filling time | 20 | min |
| Light phase | 715 | ml |
| Heavy phase | 210 | ml |

| | |
|---|---|
| Antibody content | 4.8 g/l (cell culture) |

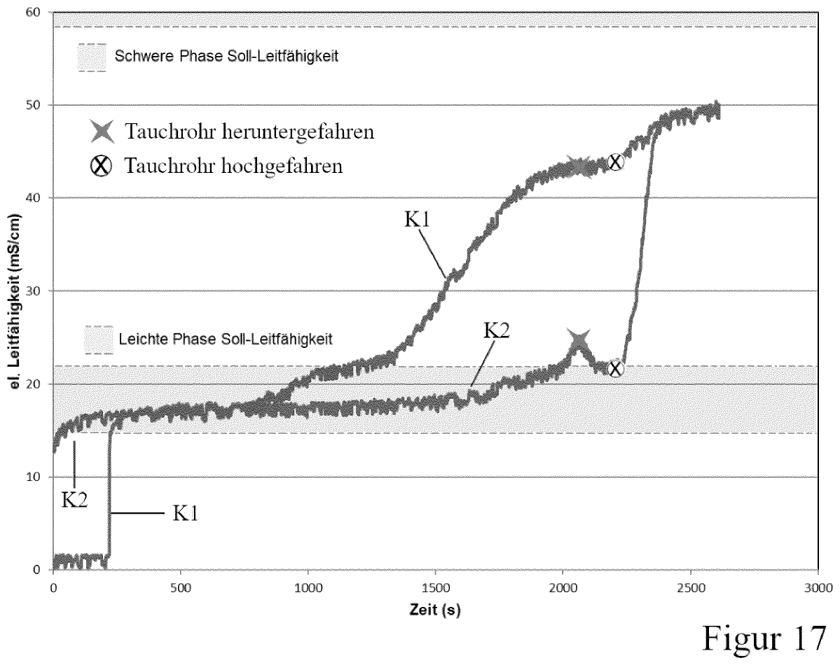

The recorded electrical conductivities of Example 5.3 using two probes, one probe for the heavy phase (e.g., probe 160.1 in FIG. 2) and one probe for the light phase (e.g., probe 160.2 in FIG. 2), are plotted against time in [s] in FIG. 17.

As can be seen in FIG. 17 and the measured curves K1 and K2, the courses of the conductivity measured values are flatter due to the lower adjusted mass flow rate, but all characteristic features can be recognized.

FIG. 17 shows that the values for the electrical conductivity of the first probe in curve K1 increase abruptly from 0 mS/cm to 13 mS/cm. Electrical conductivity then increases to 16-17 mS/cm and is then detected constantly. The second probe (curve K2) shows the same measured value as the first probe, but with a slight time delay. After both probes have detected the light phase, the measured value for the electrical conductivity of the first probe increases (curve K1) and reaches its maximum at 50 mS/cm, wherein a first asymptotic course is recognizable from a measured value of 43 mS/cm. Therefore, the first probe actually detects the intermediate phase (in the present case, heavy phase with cells) with an electrical conductivity in the range of 40-50 mS/cm from about 40 mS/cm. Since no heavy phase was measured at 60-80 mS/cm, it is assumed that all of the heavy phase has effectively transitioned to intermediate phase or that the conductivity probe is only measuring intermediate phase. Therefore, the intermediate phase in this example corresponds to the heavy phase.

The measured value for the electrical conductivity of the second probe (curve K2) remains at the constant value of 15 to 16 mS/cm for longer and then increases very slowly to approximately 24.5 mS/cm. At such point in time, the immersion tube is briefly driven down (asterisk in curves K1 and K2 in FIG. 17) and driven up again (asterisk in the circle in curves K1 and K2 in FIG. 17). For this reason, the start-up and shutdown curves are significantly different in this case.

Eventually, both measured values increase to 50 mS/cm. As soon as the second measuring probe indicates the same value as the first, the test is terminated and the pumps are switched off.

Table 11 below shows the electrical conductivity measured values for Examples 5.2 and 5.3:

TABLE 11
Electrical conductivity for Example 5.2 and 5.3

| | |
|---|---|
| Sample | Conductivity [mS/cm] |

Reference Example 5.2 (insufficient mixing)

| | |
|---|---|
| Light phase start | 20-30 |
| Light phase minute 15 | 20-30 |
| Light phase minute 30 | 20-30 |
| Light phase pool* | 20-30 |
| Heavy phase pool* | 60-70 |

Example 5.3 (sufficient mixing)

| | |
|---|---|
| Heavy phase | 40-50 |
| Light phase start | 20-25 |
| Light phase minute 30 | 20-25 |

*Pool means the phase obtained after extraction

Continuous operation was carried out here only very briefly to demonstrate the measuring principle. This can, of course, be carried out for much longer, e.g., several hours or even longer.

In a bar graph, FIG. 18 shows the cell number in [10⁶ cells/mL] of the cell culture used in Reference Example 5.2 and Example 5.3 (11,1*10⁶ cells/ml) and the cell number of the light phase obtained during the method, which is to contain the target product.

The light phase of Reference Example 5.2. shows no depletion of cells in FIG. 18 (left side of FIG. 18). The electrical conductivity of the heavy phase obtained in Reference Example 5.2 reveals such insufficient separation by the conductivity value of 60-70 mS/cm, since this corresponds to the pure heavy phase without cells. “Pool” in FIG. 18 designates the phase obtained after extraction.

In Example 5.3 on the right side of FIG. 18, it can be seen that there are consistently fewer cells in the light phase in which the product is present than in the cell culture at any point in time during the extraction method. The reduced cell count can also be observed throughout the light phase after extraction has occurred. The successful separation of the cells can again be seen from the electrical conductivity of the heavy phase. This has a significantly reduced electrical conductivity with a conductivity range of 40-50 mS/cm, suggesting the presence of cells and other conductivity-lowering bioparticles.

Analysis shows that the antibodies are enriched in the separated light phase of Example 5.3 and are in purified form. Therefore, the method according to the invention enables the product phase (light phase) to be constantly separated from the heavy phase with the aid of conductivity measurement/phase boundary control via continuous extraction.

By reducing the volume flow, cell harvesting could be successfully carried out with the phosphate system. Thus, it was confirmed that, due to the low mixing of the system in the start-up region, there was a phase inversion of the system in Example 5.2 (reference example), which could be avoided in Example 5.3 (example according to the invention) with mixing with sufficient mass transfer.

### Example 5.4

### DN150 Scale—Aqueous Two-Phase Phosphate System

Example 5.4 shows how the automated adjustment of the height or immersion depth of the immersion tube is carried out as a function of the conductivity values determined for 2 probes. An aqueous two-phase phosphate system is used in continuous operation.

In the example shown, the target product is located in the light phase and the immersion tube in the embodiment with an overtube, which extends into the heavy phase, is to remove heavy phase and intermediate phase from the separator.

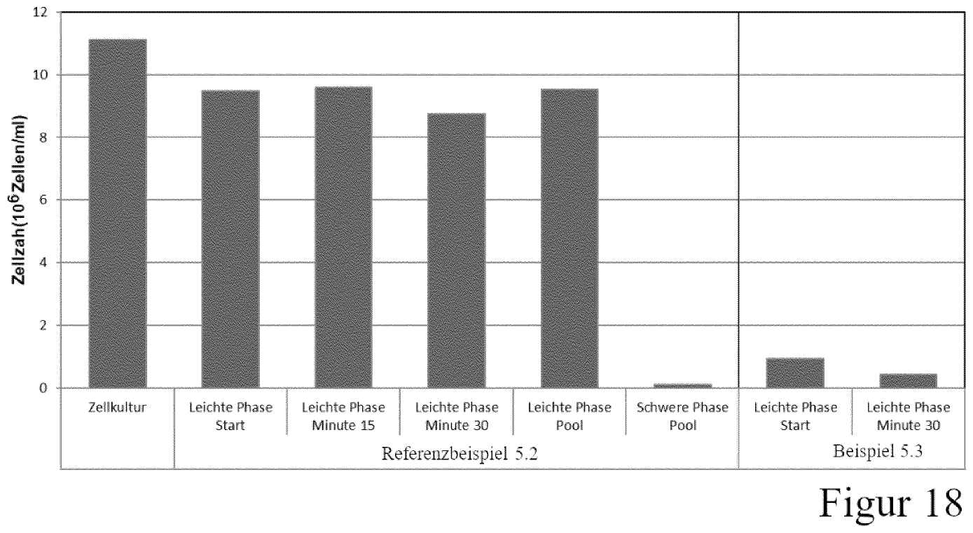

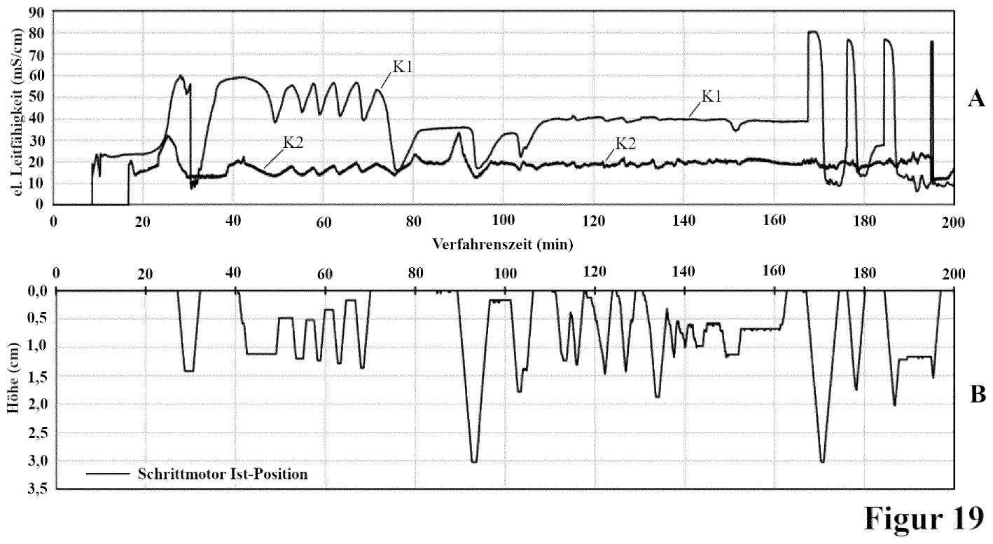

FIG. 19 shows the two electrical conductivity profiles of the two probes (FIG. 19A) and the automated height adjustment for an immersion tube (FIG. 19B) for an extraction process. Curve K1 in FIG. 19A gives the electrical conductivities measured by the first probe in the heavy phase, and curve K2 in FIG. 19A gives the electrical conductivities measured by the second probe in the light phase. FIGS. 19A and 19B are each plotted against the method time in [min].

FIG. 19B shows the driving up and down of the immersion tube based on the position of the opening in the immersion tube, which acts as an overflow valve. Therefore, the height information in FIG. 19B represents the position of the overflow valve, at the height of the opening, where the fluid flows out. At the start, the immersion tube is in its initial position at height 0 cm (along the x-axis in FIG. 19B). Therefore, the immersion tube is in the driven-up state and does not remove any phase from the separating device. After approximately 27 min, the immersion tube is then driven down to approximately 1.5 cm and then driven up again to the initial position (height 0 cm) at approximately 32 min. As can be seen, the automated control shown compensates for the deviation of the measured value of the electrical conductivity from the target value by adjusting the position of the immersion tube, i.e., the position of the opening in the immersion tube.

The light phase, represented by the electrical conductivity values of curve K2, has the conductivity of 20-30 mS/cm typical in such system throughout the test. At the start of the test, the first probe in curve K1 initially measures the electrical conductivity values for the light phase, since the separator is filled (up to approximately 25 min). Once the separator is completely filled with the system, the first probe (curve K1) measures the electrical conductivity values of the pure heavy phase (60-80 mS/cm). The immersion tube is driven down to drain heavy phase until the intermediate phase (in this case, mainly heavy phase with cells) is detected with a lower electrical conductivity in the range of 40-50 mS/cm. Therefore, the automatic adjustment of the height or immersion depth of the immersion tube selectively regulates the position of the intermediate phase in the separator. If no intermediate phase is present, this will lower the phase boundary interface between the heavy and light phases accordingly in the separator.

From approximately 50 min onwards, the characteristic alternation of detected heavy phase (deflection of curve K1 in FIG. 19A upward) and detection of the intermediate phase (deflection of curve K1 in FIG. 19A downwards) occurs upon the extraction in curve K1. This is due to the fact that continuous operation is performed, and therefore aqueous two-phase system to be separated is constantly fed into the separator. At the same time, the immersion tube is moved back and forth between the heavy phase and the intermediate phase (FIG. 19B), in order to allow the heavy phase and intermediate phase to flow out of the separator and to ensure that the first probe remains between the heavy phase and the intermediate phase. This ensures that the light phase is not lost with the heavy phase and that the intermediate phase accumulating in the separator is drained through the immersion tube at regular intervals.

Analysis shows that the antibodies are enriched in the separated light phase and are present in purified form.

### Example 6

Continuous Extraction in the Separator with a Cell Culture for Purification and Enrichment of Plasmid DNA in an Aqueous Two-Phase Citrate System

Example 6 describes continuous separator tests with cell lysate from an E. coli fermentation. The cell lysate is obtained via alkaline lysis, and the subsequent necessary neutralization is carried out with the salt buffer also required for aqueous two-phase extraction (ATPE) (in this example: citrate). Advantageously, the salt quantity required for the APTE can be added completely in the lysis step.

In contrast to Examples 5.1 to 5.4 described previously, the target component in Example 6 is plasmid DNA (pDNA) rather than a monoclonal antibody. In addition, such system has a distribution of the target component into the heavy phase; i.e., the plasmid DNA is enriched in the heavy phase after mixing with sufficient mass transfer to be separated from the rest. Thereby, the intermediate phase represents a mixture of non-coalesced droplets, precipitates bound in between, and digested E. coli cells. The two-phase system used employs a polyethylene glycol with an average molecular mass of 1450 g/mol (PEG1450) instead of polyethylene glycol with an average molecular mass of 400 g/mol (PEG400) for two-phase formation. Similar to the previous examples, a polymer-rich light phase and a salt-rich heavy phase are formed. Target and minor components are distributed in such multiphase system such that the target product has been enriched in and is substantially present in the heavy phase, and the undesired components have been enriched in and are substantially present in the intermediate and light phases.

Thus, electrical conductivity detection can again be used to separate the target product in the target phase (in this example, the heavy phase) from the counterphase (in this example, the light phase) and the intermediate phase with cell lysate.

For the continuous extraction carried out in Example 6 according to the method of the invention, a two-phase citrate system is used to purify and enrich the target product, in this case plasmid DNA, from a cell culture. The initial weight of phase-forming components and cell culture along with the adjusted mass fractions are summarized in Table 12:

TABLE 12
Initial weight and mass fractions for Example 6

| | Initial weight | Actual mass fraction |
|---|---|---|
| | [g] | [wt. %] |
| Cell lysate | 4406.4 | 40.8 |
| Citrate buffer (35 wt. %, pH 6.0) | 2721.6 | 25.2 |
| PEG 1450 (60 wt. %) | 3672 | 34.0 |

The aqueous two-phase citrate system has already been suitably mixed in a static mixer, such that the components partition between the phases. The target product accumulates in the heavy phase in Example 6. The aqueous two-phase citrate system is conveyed via a pump into the separator at a mass flow rate of 60 g/min. With such conveying rate, it takes approximately 10 minutes for the light phase to exit at the top of the outlet.

The test overview for Example 6 is given in Table 13 below.

TABLE 13
Test overview for Example 6

| | | |
|---|---|---|
| Mass flow rate | 60 | g/min |
| Total duration | 180 | min |
| Filling time | 10 | min |
| Heavy phase | 2300 | ml |
| Light phase | 7100 | ml |
| pDNA content | 2.43 | mg/g WCP |

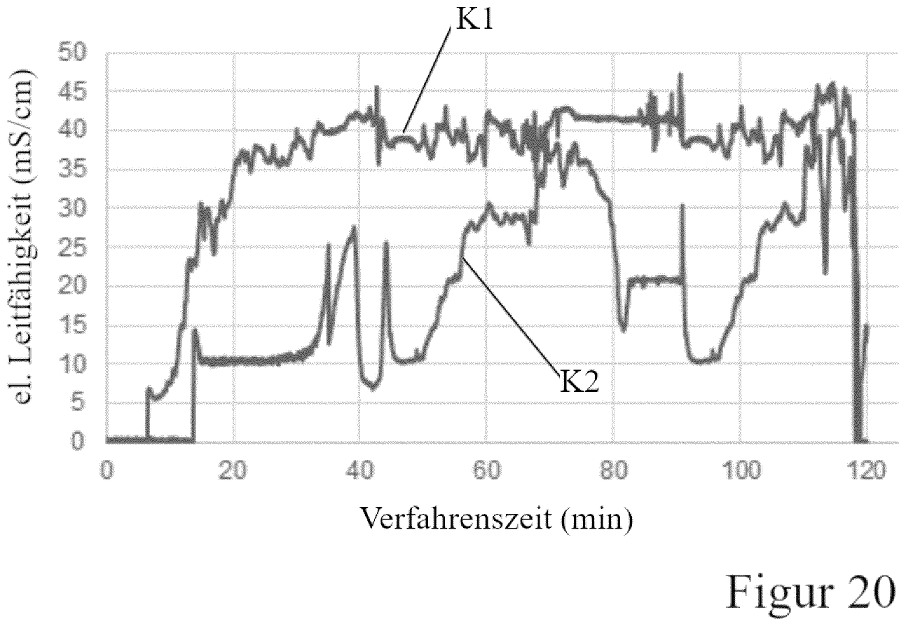

The measured electrical conductivities of Example 6 are plotted against the method time in [min] in FIG. 20. Upon the start-up of the continuous extraction, the typical course can be seen again. The first probe (curve K1) is the first to show the electrical conductivity values for the light phase at approximately 5-10 mS/cm. The curve K1 then increases almost constantly up to approximately 40 mS/cm. At approximately 40 mS/cm, a flattening of the slope can be seen. Here, the intermediate phase with biomass/cell lysate is detected. The second probe (curve K2) shows a similar course upon start-up. The electrical conductivity suddenly jumps to 10 mS/cm. Light phase is detected here for the first time. From minute 35, it can be seen that the measured values for electrical conductivity differ from those in examples 5.1 to 5.4. At approximately 35 minutes, the intermediate phase with the cells is detected in curve K2. As soon as curve K2 reaches a conductivity value of more than 25 mS/cm, the immersion tube is driven down (not shown), which is again used as an overflow valve. The immersion tube is used to remove light phase and intermediate phase.

The electrical conductivity measured at the second probe (curve K2) starts to decrease as the position of the intermediate phase in the separator decreases due to the change of the immersion tube position and thus the light phase (without biomass and heavy phase droplets) is detected. In the course of the method, such control of the position of the intermediate phase (and thus also the position of the two phase boundaries to the heavy and light phase) in the separator becomes necessary twice more (at minute 70 and at minute 110) (not explicitly shown in FIG. 20).

It should be noted in this connection that the ranges for electrical conductivities given in Table 1 are for guidance only and may also lie in ranges deviating from this, as in the present example. Thus, the target value range for the electrical conductivities for the heavy phase here is from approximately 40 mS/cm, for the light phase from approximately 10 mS/cm and the intermediate phase from approximately 25 mS/cm. However, this is not problematic, since the composition of the individual phases can be determined by analyzing them. Moreover, in the present example, the separation of the phases is possible in a simple manner, since the difference in electrical conductivities between the heavy phase on the one hand and the intermediate phase and light phase on the other hand is relatively large, and the target product is present in the heavy phase.

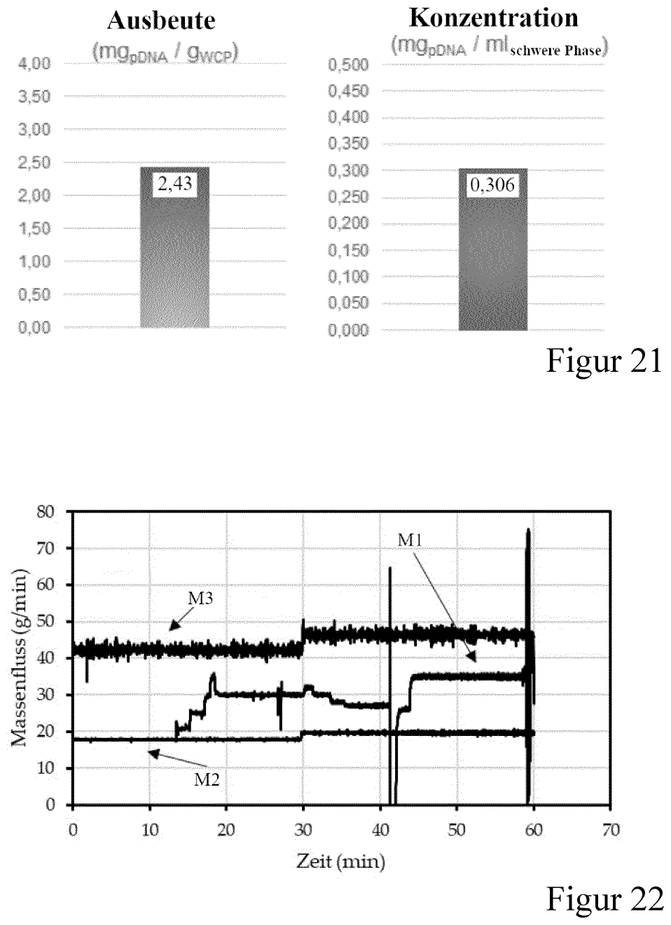

The yield of the method described in Example 6 is shown in FIG. 21 and amounts to 2.43 mg pDNA/g_{WCP} (wet cell paste), which is within the typical range of 2-3 mg/g for pDNA processes. The aqueous two-phase system (ATPS) used results in an expected increase of concentration by a factor of 3 (batch experiment). This change in concentration influenced by two-phase extraction (ATPE) could also be achieved in the continuous method with the system used. Thus, Example 6 also demonstrated that there is no loss of heavy target phase due to the controlled position of the intermediate phase in the separator.

### Example 7

Course of a continuous extraction in a separator in an aqueous two-phase citrate system using two pumps

The procedure is analogous to Example 6, but the position of the intermediate phase is controlled by two pumps. To adjust the position of the intermediate phase in the separator, the conductivity profiles are used again.

Therefore, a continuous extraction is carried out according to the method according to the invention using a two-phase citrate system to purify and enrich the target product, in this case plasmid DNA, from a cell culture. The target product is substantially in the heavy phase. The initial weight of phase-forming components and cell culture along with the adjusted mass fractions are summarized in Table 14:

TABLE 14
Initial weight and mass fractions for Example 7

| | Initial weight | Actual mass fraction |
|---|---|---|
| | [g] | [wt. %] |
| Cell lysate (neutralized) | 2764.69 | 70 |
| PEG 1450 (60 wt. %) | 1122 | 30 |

The cell lysate solution used in Example 7 is already neutralized with citrate buffer (35 wt. %, pH 6.0). The ratio of non-neutralized cell lysate to buffer is approximately 1.6 (cell lysate:citrate buffer).

The test overview for Example 7 is given in Table 15 below.

TABLE 15
Test overview for Example 7

| | | |
|---|---|---|
| Mass flow rate | 60 | g/min |
| Total duration | 60 | min |
| Filling time | 10 | min |
| Heavy phase | 1232 | ml |
| Light phase | 2300 | ml |

In contrast to Example 6, where the removal of the heavy phase is controlled by adjusting the height of an immersion tube with an opening that acts as an overflow valve, in Example 7 the heavy phase is actively conveyed out of the separator by a pump. Likewise, the light phase is conveyed out of the separator via a second pump.

To ensure that only pure heavy or light phase is removed from the separator, the conductivity profiles are again used—as already explained in the previous examples.

If electrical conductivity at the first probe for measuring the electrical conductivity of the heavy phase drops below a predefined limit value (curve M1), the conveying speed of the “heavy phase” pump is throttled. If, in turn, the electrical conductivity of the second probe for measuring the electrical conductivity of the light phase (curve M2) rises above a predefined limit value, the conveying speed of the “heavy phase” pump is increased.

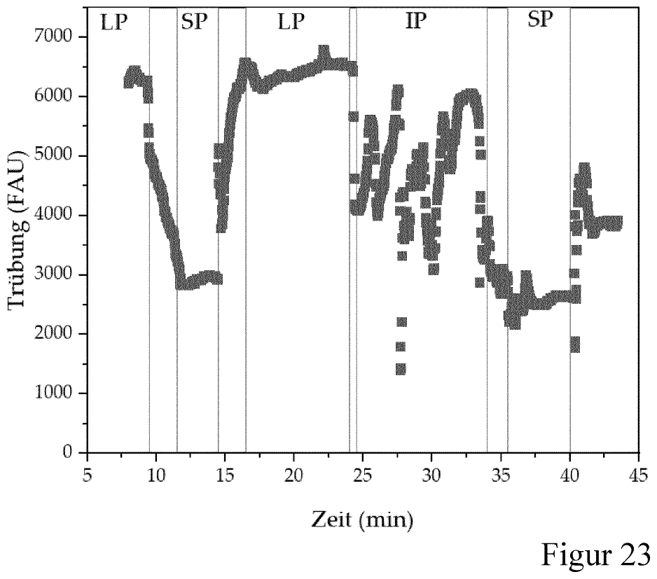

FIG. 22 shows the pump flows during the method.

The labeling of FIG. 22 is as follows:
Curve M1 . . . mass flow of heavy phase;
Curve M2 . . . mass flow of light phase (PEG1450) and
Curve M3 . . . mass flow of cell lysate.

### Example 8

Extraction of a batch with a starting solution for purification and enrichment of virus capsids in aqueous two-phase system in the separator. The extraction is carried out as described in FIG. 1.

As an example, two deposition tests with virus capsids of adeno-associated viruses (AAV) from a cell culture are carried out. In the examples described with virus capsids, the target component is enriched once in the upper light phase (Example 8.1) and once in the lower heavy phase (Example 8.2).

In contrast to the previously described Examples 5.1 to 7, a viral capsid of an adeno-associated virus is used for the target component instead of a monoclonal antibody or plasmid DNA. Furthermore, a polyethylene glycol with an average molecular weight of 8000 g/mol and an ammonium sulfate buffer (see Example 8.2) are used.

### Example 8.1

The system used in Example 8.1 produces a distribution of the target component into the light phase; i.e., the virus capsids are enriched in the light phase after mixing with sufficient mass transfer, in particular substantially in this phase, which is to be separated from the remainder.

In the two-phase system used, polyethylene glycol with an average molecular mass of 400 g/mol (PEG400) and a phosphate buffer are used for two-phase formation. Similar to the previous examples, a polymer-rich light phase and a salt-rich heavy phase are formed. The components are distributed in such multiphase system in such a way that the target product, here: adeno-associated viruses, is enriched in the light phase, in particular is substantially present therein, and undesirable components are enriched in the heavy phase, in particular are substantially present therein.

The initial weight of phase-forming components and virus starting solution along with the adjusted mass fractions are summarized in Table 16:

TABLE 16
Initial weight and mass fractions for Example 8.1

| | Initial weight | Actual mass fraction |
|---|---|---|
| | [g] | [wt. %] |
| Virus solution | 4.405 | 44.05 |
| Phosphate buffer | 3.875 | 38.75 |
| (40 wt. %, pH 8.0) | | |
| PEG 400 | 1.720 | 17.20 |

Thereby, the aqueous two-phase phosphate system is suitably mixed in the separation vessel, such that the components may partition between the phases. The target product accumulates in the light phase at a conductivity of 6.24 mS/cm in Example 8.1. The conductivity of the heavy phase is 78.7 mS/cm.

### Example 8.2

The system used in Example 8.2 produces a distribution of the target component in the heavy phase; i.e., the virus capsids are enriched in the heavy phase after mixing with sufficient mass transfer, in particular substantially in this phase, which is to be separated from the remainder.

The two-phase system employed uses polyethylene glycol with an average molecular mass of 8000 g/mol (PEG8000) and ammonium sulfate buffer for two-phase formation. Similar to the previous examples, a polymer-rich light phase and a salt-rich heavy phase are formed. The components are distributed in such multiphase system in such a way that the target product, here: adeno-associated viruses, is enriched in the heavy phase, in particular is substantially present in this phase, and undesirable components are enriched in the light phase, in particular are substantially present in this phase.

The initial weight of phase-forming components and cell culture along with the adjusted mass fractions are summarized in Table 17. The virus solution had an initial concentration of 2.17×10\hat{ }9 capsid/ml.

TABLE 17
Initial weight and mass fractions for Example 8.2

| | Initial weight | Actual mass fraction |
|---|---|---|
| | [g] | [wt. %] |
| Virus solution | 1.40 | 14 |
| Ammonium sulfate buffer | 6.60 | 66 |
| (20 wt. %, pH 8.0) | | |
| PEG8000 (50 wt. %) | 2.0 | 20 |

Thereby, the aqueous two-phase ammonium system is suitably mixed in the separator, such that the components partition between the phases. The target product accumulates in the lower heavy phase in Example 8.2. The conductivity of the lower heavy phase is 175.2 mS/cm. The conductivity of the upper light phase is 10.4 mS/cm.

The virus solution had an initial concentration of 2.17×10\hat{ }9 capsid/ml in the extraction tests of Examples 8.1 and 8.2, carried out in one batch each. The capsids were able to be enriched completely in the target phases. No virus capsid could be determined in the counterphase.

Due to the differences in conductivity of the light and heavy phases, the virus-containing solutions could also be separated by means of conductivity detection and transfer to separation vessels in continuous operation (see Table 18)

TABLE 18

| System | | AAV ELISA | Conductivity |
|---|---|---|---|
| Example | Phase | Capsids/mL | [mS/cm] |
| 8.1 | Light phase | 3.86 × 10\hat{ }9 | 6.24 |
| | Heavy phase | <LLD* | 78.7 |
| 8.2 | Light phase | <LLD* | 10.4 |
| | Heavy phase | 2.36 × 10\hat{ }9 | 175.2 |

*. . .″<LLD″ means that the capsid content is below the lower limit of detection (LLD).

### Example 9

Course of a Continuous Extraction in a Separator in an Aqueous Two-Phase system using a turbidity probe

To illustrate the implementation of the method with an alternative measuring probe, the system described in Example 6 is used with a turbidity probe. In this exemplary embodiment, only one probe is used instead of two probes. The position of the probe is changed at time intervals, such that the turbidity of the other phases can also be measured. In other words, the probe can be driven up and down to be able to measure the individual phases. FIG. 23 shows the time recording of the measurement, in order to illustrate the measuring principle in detail. In FIG. 23, LP means light phase, SP means heavy phase and IP means intermediate phase. The free regions marked by vertical lines in FIG. 23 between the measurement of the light phase, heavy phase and intermediate phase represent the transitions in which the single probe is switched over, i.e., driven up or down.

In FIG. 23, the measurement of turbidity in the light phase (LP) is started. After 9 minutes, the position of the probe is displaced downward, such that the turbidity of the heavy phase (SP) can be measured. Such method can be repeated as often as required, such that the properties of two or three phases can also be detected at short intervals with just one probe. In the upper region of FIG. 23, there is a high (strong) turbidity, in the lower region a low turbidity. Therefore, the light phase LP shows high turbidity, whereas the heavy phase SP shows low turbidity. The intermediate phase IP shows overlapping turbidity characteristics.

Therefore, FIG. 23 serves for orientation and illustration of the measuring principle. For the system used in this example, it can therefore be shown that turbidity is also a suitable indicator for distinguishing between two or three phases. A separation of the phases, based on the turbidity measurement with one or even more probes, can therefore be readily carried out over a suitable period of time.

The invention comprises aspects disclosed in the following clauses, which form part of the description but are not claims:

### CLAUSES

1. A method for purifying and enriching a target product selected from the group consisting of
immunoglobulins or other proteins; or
plasmid DNA, genomic DNA, RNA or other nucleic acids; or
viruses,
comprising the following steps:
a. Providing a starting solution containing the target product;
b. Converting the starting solution into an aqueous two-phase system by adding at least one polymer and at least one salt in a suitable concentration, or by adding at least two polymers in a suitable concentration;
c. Mixing the aqueous two-phase system while maintaining a sufficient mass transfer, such that the target product is enriched in the light phase (20, 120) or in the heavy phase (40, 140);
d. Allowing the phases to segregate in a separating device (10, 100) into a light phase (20, 120), a heavy phase (40, 140) and, if an intermediate phase is present, an intermediate phase (30, 130);
e. Continuously measuring in at least one of the phases in the separating device (10, 100) to determine the position of the two phases (20, 120, 40, 140) and the phase boundary interface or, in the presence of an intermediate phase (30, 130), the position of the intermediate phase (30, 130) and the two phase boundary interfaces;
f. Removing each of the light phase (20, 120), the heavy phase (40, 140) and, in the presence of an intermediate phase (30, 130), the intermediate phase from the separating device (10, 100) based on the measured electrical conductivity of at least one of the phases; and
g. Extracting the phase with the target product.

2. The method according to clause 1,
characterized in that
the continuous measurement of the electrical conductivity in at least one of the phases in the separating device (10, 100) is carried out with one or more conductivity measuring devices in the form of one, two, three or more probes (60, 160.1, 160.2, 162.1, 162.2, 162.3, 162.4, 163.1, 163.2, 163.3, 163.4).

3. The method according to clause 1 or 2,
characterized in that
at least one probe (160.1, 162.1, 163.1) is used for continuously measuring the electrical conductivity of the heavy phase (140) and at least one probe (160.2, 162.3, 162.4, 163.3, 163.4) is used for continuously measuring the electrical conductivity of the light phase (20, 120) at a suitable position in the separating device (100).

4. The method according to one of the preceding clauses 1 to 3,
characterized in that
an outlet (50) is provided in the separating device (10) and the removal of the phases (10, 20, 30) from the separating device (10) through the outlet (50) according to step f. is carried out by gravity or using positive pressure or negative pressure, generated with or without a pump, or a pump;
or
an upper outlet (150.2) is provided in the separating device (100), and the removal of the light phase (120) from the upper outlet (150.2) of the separating device (100) is carried out using gravity or positive pressure or negative pressure, generated with or without a pump, or a pump,
and
a lower outlet (150.1) is provided in the separating device (100), and the removal of the heavy phase (140) from a lower outlet (150.1) of the separating device (100) is carried out using gravity or positive pressure or negative pressure, generated with or without a pump, or a pump from the separating device (100).

5. The method according to one of the preceding clauses 1 to 4,
characterized in that
the removal of a phase according to step f. is carried out in continuous operation by means of a height-adjustable immersion tube (180, 182) having an opening (190, 192) that is immersed in the phase to be removed, wherein
the immersion tube (180) is connected to the lower outlet (150.2) in such a way that the phase can flow out under gravity to the outlet (150.1) through the opening (182) in the immersion tube (180) or
the immersion tube (190) has an opening (192) through which the removal of the phase is carried out using positive pressure or negative pressure, generated with or without a pump, or a pump.

6. The method according to one of the preceding clauses 1 to 5,
characterized in that
the height-adjustable immersion tube (180, 190) is used with an overtube (183, 193) extending from top to bottom,
wherein the immersion tube (180, 190) is formed of a height-adjustable inner tube (181, 191) having an opening (182, 192) for removing one of the phases, and an overtube (183, 193) extending from top to bottom, and the length of the overtube (183) is defined by an upper end arranged above the light phase (120) and alower end (183a, 193a), wherein the diameter of the overtube (183, 193) isis adjusted to be largerlarger than the diameter of the inner tube (181, 191), such that an intermediate space between the inner tube (181, 191) and overtube (183, 193) is formed, and the length of the overtube (183) is selected such that the overtube (183, 193) projects beyond the opening (182, 192) of the inner tube (181, 191) and the lower end (183a, 193a) of the overtube (183, 193) is immersed in the heavy phase (140) or, if present, in the intermediate phase (130), such that the fluid of the phase into which the lower end (183a, 193a) of the overtube (183, 193) is immersed rises in the intermediate space and can exit through the opening (182, 192) in the inner tube (181, 191).

7. The method according to one of the preceding clauses 1 to 5,
characterized in that
the height-adjustable immersion tube (180, 190) with an overtube extending from the bottom to the top is used,
wherein the height-adjustable immersion tube is formed of a height-adjustable inner tube (181, 191), which has an opening (182, 192) for removing one of the phases and an overtube extending from bottom to top, and the length of the overtube is defined by a lower end, which is arranged below the heavy phase (140), and an upper end, wherein the diameter of the overtube is adjusted to be larger than the diameter of the inner tube, such that an intermediate space is formed between the inner tube (181, 191) and the overtube, and the length of the overtube is selected so that the overtube projects beyond the opening (182, 192) of the inner tube (181, 191) and the upper end of the overtube is immersed in the light phase (120) or, if present, in the intermediate phase (130), such that the fluid of the phase in which the upper end of the overtube is immersed flows into the intermediate space and can exit through the opening (182, 192) in the inner tube (181, 191).

8. The method according to clause 6 or 7,
characterized in that
the length of the overtube (183, 193) extending from top to bottom and its diameter are selected in such a way that the entry of suspended matter, flocs and other solid or semi-solid components, into the intermediate space between the inner tube (181, 191) and the overtube (183, 193) is prevented by the overtube (183, 193);
or
the length of the overtube extending from bottom to top and its diameter are selected in such a way that the entry of suspended matter, flocs and other solid or semi-solid components into the intermediate space between the inner tube (181, 191) and the overtube is prevented by the overtube.

9. The method according to one of the preceding clauses 1 to 8,
characterized in that
the probe(s) (160.2, 162.3, 162.4, 163.3, 163.4) for the light phase (120) is (are) positioned lower than the upper outlet (150.2) in the separating device (100) and the probe(s) (160.1, 162.1, 163.1) for the heavy phase (140) is (are) positioned higher than the lower outlet (150.1) in the separating device (100);
and/or
an immersion tube (180, 190) with or without an overtube (183, 193) is used, wherein, when using an immersion tube (180, 190) without an overtube (183, 193), the opening (182, 192) of the immersion tube (180, 190) for removing the heavy phase (140) is located lower than the probe(s) (160.1, 162.1, 163.1) in the heavy phase (140) and wherein the opening (182, 192) of an immersion tube (180, 190) for removing the light phase (120) is located higher than the probe(s) (160.2, 162.3, 162.4, 163.3, 163.4) in the light phase (120); and
wherein, when using an immersion tube (180, 190) with an overtube (183, 193) extending from top to bottom, the lower end (183a, 193a) of the overtube (183, 193) is located lower than the probe(s) (160.1, 162.1, 163.1) in the heavy phase (140) for removing the heavy phase (140) when the heavy phase (140) is removed through the opening (182, 192) of the immersion tube (180, 190); and wherein, when using an immersion tube with an overtube extending from the bottom to the top, when the light phase (120) is removed through the opening (182, 192) of the immersion tube, the upper end of the overtube for removing the light phase (120) is located higher than the probe(s) (160.2, 162.3, 162.4, 163.3, 163.4) in the light phase (120).

10. The method according to one of the preceding clauses 1 to 9,
characterized in that
continuously measuring the electrical conductivity of the light phase (120) in the separating device (100) in step e. for one or more probes (160.2, 162.3, 162.4, 163.3, 163.4) in each case produces a value for electrical conductivity, which is compared with a target value range for the electrical conductivity of the light phase (120) and, if the target value range is exceeded, heavy phase (140) is removed, wherein the removal quantity, removal rate and/or removal duration for the heavy phase (140) are controlled in such a way that the measured conductivity of the light phase (120) is again in the target value range;
or
continuously measuring the electrical conductivity of the heavy phase (140) in the separating device (100) in step e. for one or more probes (160.1, 162.1, 163.1) in each case produces a value for electrical conductivity, which is compared with a target value range for the electrical conductivity of the heavy phase and, if the electrical conductivity falls below the target value range, less heavy phase (140) is removed, wherein the removal quantity, removal rate and/or removal duration for the heavy phase (140) are controlled in such a way that the measured conductivity of the light phase (120) is again in the target value range.

11. The method according to one of the preceding clauses 1 to 10,
characterized in that
the electrical conductivity values are used to vary the position of the phase(s) and phase boundary interface(s) in continuous operation by monitoring and controlling one or more of the following:
   the removal rate for the heavy phase being increased or decreased;
   the removal rate for the light phase being increased or decreased;
   the removal quantity for the heavy phase being increased or decreased;
   the removal quantity for the light phase being increased or decreased;
   the removal duration for the heavy phase being increased or decreased;
   the removal duration for the light phase being increased or decreased;
   the removal of the light or heavy phase being interrupted;
   the feed quantity of the aqueous two-phase system flowing into the separating device (100), with or without the target product, being increased or decreased, wherein the aqueous two-phase system without the target product is a flushing solution;
   the feed rate of the aqueous two-phase system flowing into the separating device, with or without a target product, being increased or decreased, wherein the aqueous two-phase system without a target product is a flushing solution; and/or
   the immersion tube being driven up or down to allow to flow out or suck away one or two phases and, if necessary, repositioned upward or downward;
wherein the one or more removal devices and/or means may be used to carry out the preceding measures:
   gravity;
   positive pressure;
   negative pressure;
   one or more pumps;
and/or
   removal devices for controlled draining or interrupting the removal of phases at the outlet, selected from continuously adjustable valves with different degrees of opening, adjustable hose pinch clamps, adjustable shut-off valves or adjustable flow limiters.
12. The method according to one of the preceding clauses 1 to 11,
characterized in that
for continuously measuring the electrical conductivity of the phases in the separating device (10, 100), two, three, four or more probes (160.1, 160.2, 162.1, 162.2, 162.3, 162.4, 163.1, 163.2, 163.3, 163.4) are each inserted into and used in an insertion opening (157.1, 157.2, 158.1, 158.2, 158.3, 158.4, 159.1, 159.2, 159.3, 159.4) in the wall (155) of the separating device (100), wherein the insertion openings (157.1, 157.2, 158.1, 158.2, 158.3, 158.4, 159.1, 159.2, 159.3, 159.4) have an arrangement that is selected from:
   an axial arrangement of the insertion openings (157.1, 157.2), wherein the insertion openings (157.1, 157.2) are arranged on a straight connecting line;
   a radial arrangement of the insertion openings (158.1, 158.2, 158.3, 158.4), wherein the insertion openings (158.1, 158.2, 158.3, 158.4) are arranged in an arc; or
   a radially offset arrangement of the insertion openings (159.1, 159.2, 159.3, 159.4), wherein the insertion openings (159.1, 159.2, 159.3, 159.4) are arranged in an arc and additionally shifted in a preferred direction.

13. The method according to one of the preceding clauses 1 to 12, characterized in that
the mixing of the aqueous two-phase system is carried out while maintaining a sufficient mass transfer by using active mixing and/or static mixing.

14. The method according to one of the preceding clauses 1 to 13,
characterized in that at least one of the following conditions is satisfied:
   the method is carried out continuously or discontinuously;
   a cell culture, cell culture supernatant or cell lysate containing the target product is used as the starting solution;
   an aqueous two-phase system is prepared with the starting solution, with which a phase ratio of the phase containing the target product to the counterphase of >1.1 is present;
   a density ratio of light phase (20, 120) to heavy phase (40, 140) of at least 1.06 is used;
   in the aqueous two-phase system, polyethylene glycol (PEG), polypropylene glycol (PPG), dextran, PEG-PPG-PEG copolymer (EOPO) or mixtures of these are used as the polymer or as one of the polymers;
   in the aqueous two-phase system, polyethylene glycol with a molecular weight between 200 and 8000 g/mol is used as the polymer;
   in the aqueous two-phase system, at least one polymer is used in a concentration of from 5 to 35 wt. %, based on the total quantity of aqueous two-phase system;
   in the aqueous two-phase system, a phosphate salt, ammonium salt, potassium salts, acetate salts, sodium salts or citrate salt or mixtures of these are used as salt or as one of the salts;
   instead of a salt, an acid selected from citric acid or acetic acid is used;
   stabilizing components selected from amino acids, sugars or other stabilizing additives are added to the aqueous two-phase system;
   the method is carried out continuously, but steps a. and/or d. are carried out semi-continuously.

15. The method according to one of the preceding clauses 1 to 14,
characterized in that
at the end of the extraction method for the residual discharge of a light phase (120) containing the target product, a flushing solution (115) is added to the separating device (100), which displaces the light phase (120) containing the target product from the separating device (100) and the latter can thus be removed through the outlet (150.2) arranged at the top,
and/or
at the end of the extraction method for the residual discharge of a light phase (120) containing the target product, no more heavy phase (140) is removed, the latter thereby accumulates and displaces the light phase (120) upward, such that the light phase (120) can be removed through the outlet (150.2) arranged at the top.

16. The method according to one of the preceding clauses 1 to 14,
characterized in that
at the end of the extraction method for the residual discharge of a heavy phase (140) containing the target product, a flushing solution (115) is added to the separating device (100), which displaces the heavy phase (140) containing the target product from the separating device (100) and the latter can thus be removed through the outlet (150.1, 150.3) arranged at the bottom,
and/or
at the end of the extraction method for residual discharge of a heavy phase (140) containing the target product, no more light phase (120) is removed, the latter thereby accumulates and displaces the heavy phase (140) downward, such that the heavy phase (140) can be removed through the outlet (150.1, 150.3) arranged at the bottom.

17. A device for carrying out the method according to one of the clauses 1 to 16, comprising
a separating device (10, 100) for separating the phases;
at least one outlet (50, 150.1, 150.2) in the separating device (10, 100);
at least one probe (60, 160.1, 160.2, 162.1, 162.2, 162.3, 162.4, 163.1, 163.2, 163.3, 163.4) for measuring electrical conductivity in at least one of the phases in the separating device (10, 100);
one or more removal device(s) and/or means for regulating and/or controlling the removal quantity, removal rate and/or removal duration of the light phase (20, 120), the heavy phase (40, 140) and, if present, the intermediate phase (30, 130), based on the measured values of electrical conductivity in at least one phase.

18. A use of the method according to one of the clauses 1 to 16 or the device according to clause 17 for adjusting the position of the phases in a separating device (10, 100) and separating the phases based on the measured electrical conductivity values of at least one of the phases.

LIST OF REFERENCE SIGNS:

| | |
|---|---|
| 10, 100 | Separating device |
| 15 | Agitator shaft |
| 20, 120 | Light phase |
| 25, 125 | Motor |
| 30, 130 | Intermediate phase |
| 40, 140 | Heavy phase |
| 50, 150.1, 150.2 | Outlet |
| 60 | Probe for measuring electrical conductivity |
| 110 | ATPS solution to be separated |
| 115 | Flushing solution |
| 120a, 120c | Curve for the electrical conductivity of the |
| | light phase |
| 120b, 120d | Curve for the turbidity of the light phase |
| 130c | Curve for the electrical conductivity of the |
| | intermediate phase |
| 135 | Controller |
| 140a, 140c | Curve for the electrical conductivity of the |
| | heavy phase |
| 140b, 140d | Curve for the turbidity of the heavy phase |
| 150.1, 150.2, 150.3 | Outlet |
| 155 | Wall of the separating device |
| 157.1, 157.2, 158.1, 158.2, | Insertion openings for the probes |
| 158.3, 158.4, 159.1, 159.2 | Insertion openings for the probes |
| 159.3, 159.4 | Insertion openings for the probes |
| 160.1 | Probe for the heavy phase |
| 160.2 | Probe for the light phase |
| 162.1, 162.2, 162.3, 162.4 | Probes for measuring electrical conductivity |
| 163.1, 163.2, 163.3, 163.4 | Probes for measuring electrical conductivity |
| 180, 190 | Immersion tube |
| 181, 191 | Inner tube |
| 182, 192 | Opening |
| 183, 193 | Overtube or cladding tube |
| 183a, 193a | End of the overtube |
| 189, 199 | Height adjustability of the immersion tube |
| 200, 202 | Graph |
| I | Start-up |
| II | Continuous operation |
| III | Shutdown |

### BIBLIOGRAPHY

1. Sommerfeld, S.; Strube, J. Challenges in biotechnology production—Generic methods and method optimization for monoclonal antibodies. Chemical Engineering and Processing: Process Intensification 2005, 44, 1123-1137, doi:10.1016/j.cep.2005.03.006;
2. Gronemeyer, P.; Ditz, R.; Strube, J. Trends in Upstream and Downstream Process Development for Antibody Manufacturing. Bioengineering 2014, 1, 188-212, doi:10.3390/bioengineeing1040188;
3. Urthaler, J.; Buchinger, W.; Necina, R. Improved downstream method for the production of plasmid DNA for gene therapy, Acta Biochim Pol 2019, 52, 703-711, doi:10.18388/abp.2005_3434;
4. Merten, Otto-Wilhelm, et al, 26: Production and purification of viral vectors and safety considerations related to their use, Advanced Textbook on Gene Transfer, Gene Therapy and Genetic Pharmacology: Principles, Delivery and Pharmacological and Biomedical Applications of Nucleotide-Based Therapies, 2014, 493-515;
5. Luechau, F.; Ling, T. C.; Lyddiatt, A. A descriptive model and methods for up-scaled method routes for interfacial partition of bioparticles in aqueous two-phase systems. Biochemical Engineering Journal 2010, 50, 122-130, doi:10.1016/j.bej.2010.04.006;
6. Leonard, R.A. Recent Advances in Centrifugal Contactor Design. Separation Science and Technology 1988, 23, 1473-1487, doi:10.1080/01496398808075643;
7. WO 2014/135420 A1;
8. Frerix, A., et al., Scalable recovery of plasmid DNA based on aqueous two-phase separation, Biotechnology and Applied Biochemistry, 2005, 42(1): pp. 57-66;
9. DE 44 00 397 A1;
10. DE 199 23 901 A1;
11. KR 101 143 889 B1;
12. U.S. Pat. No. 3,220,930 A;
13. EP 1 059 105 A2 and
14. EP 1 762 616 A1.

1. A method for purifying and enriching a target product selected from the group consisting of
immunoglobulins or other proteins; or
plasmid DNA, genomic DNA, RNA or other nucleic acids; or
viruses,
comprising the following steps:
a. Providing a starting solution containing the target product;
b. Converting the starting solution into an aqueous two-phase system by adding at least one polymer and at least one salt in a suitable concentration, or by adding at least two polymers in a suitable concentration;
c. Mixing of the aqueous two-phase system while maintaining a mass transfer, such that the target product is enriched in the light phase (20, 120) or in the heavy phase (40, 140);
d. Allowing the phases to segregate in a separating device (10, 100) into a light phase (20, 120), a heavy phase (40, 140) and, if an intermediate phase is present, an intermediate phase (30, 130);
e. Continuously measuring the electrical conductivity in at least one of the phases in the separating device (10, 100) to determine the position of the two phases (20, 120, 40, 140) and the phase boundary interface or, in the presence of an intermediate phase (30, 130), the position of the intermediate phase (30, 130) and the two phase boundary interfaces;
f. Removing each of the light phase (20, 120), the heavy phase (40, 140) and, in the presence of an intermediate phase (30, 130), the intermediate phase from the separating device (10, 100) based on the continuously measured electrical conductivity of at least one of the phases in step e.; and
g. Extracting the phase with the target product.

2. The method according to claim 1,
characterized in that
the continuous measurement of the electrical conductivity in at least one of the phases in the separating device (10, 100) is carried out with one or more conductivity measuring devices in the form of one, two, three or more probes (60, 160.1, 160.2, 162.1, 162.2, 162.3, 162.4, 163.1, 163.2, 163.3, 163.4),
in particular, at least one probe (160.1, 162.1, 163.1) is used for continuously measuring the electrical conductivity of the heavy phase (140) and at least one probe (160.2, 162.3, 162.4, 163.3, 163.4) is used for continuously measuring the electrical conductivity of the light phase (20, 120) at a suitable position in the separating device (100).

3. The method according to one of the preceding claims 1 to 2,
characterized in that
an outlet (50) is provided in the separating device (10) and the removal of the phases (10, 20, 30) from the separating device (10) through the outlet (50) according to step f. is carried out by gravity or using positive pressure or negative pressure, generated with or without a pump, or a pump;
or
an upper outlet (150.2) is provided in the separating device (100), and the removal of the light phase (120) from the upper outlet (150.2) of the separating device (100) is carried out using gravity or positive pressure or negative pressure, generated with or without a pump, or a pump,
and
a lower outlet (150.1) is provided in the separating device (100), and the removal of the heavy phase (140) from a lower outlet (150.1) of the separating device (100) is carried out using gravity or positive pressure or negative pressure, generated with or without a pump, or a pump from the separating device (100).

4. The method according to one of the preceding claims 1 to 3,
characterized in that
the removal of a phase according to step f. is carried out in continuous operation by means of a height-adjustable immersion tube (180, 182) having an opening (190, 192) that is immersed in the phase to be removed, wherein
the immersion tube (180) is connected to the lower outlet (150.2) in such a way that the phase can flow out under gravity to the outlet (150.1) through the opening (182) in the immersion tube (180) or
the immersion tube (190) has an opening (192) through which the removal of the phase is carried out using positive pressure or negative pressure, generated with or without a pump, or a pump.

5. The method according to one of the preceding claims 1 to 4,
characterized in that
the height-adjustable immersion tube (180, 190) is used with an overtube (183, 193) extending from top to bottom,
wherein the immersion tube (180, 190) is formed of a height-adjustable inner tube (181, 191) having an opening (182, 192) for removing one of the phases, and an overtube (183, 193) extending from top to bottom, and the length of the overtube (183) is defined by an upper end arranged above the light phase (120) and a lower end (183a, 193a), wherein the diameter of the overtube (183, 193) is adjusted to be larger than the diameter of the inner tube (181, 191), such that an intermediate space between the inner tube (181, 191) and overtube (183, 193) is formed, and the length of the overtube (183) is selected such that the overtube (183, 193) projects beyond the opening (182, 192) of the inner tube (181, 191) and the lower end (183a, 193a) of the overtube (183, 193) is immersed in the heavy phase (140) or, if present, in the intermediate phase (130), such that the fluid of the phase into which the lower end (183a, 193a) of the overtube (183, 193) is immersed rises in the intermediate space and can exit through the opening (182, 192) in the inner tube (181, 191).
or
the height-adjustable immersion tube (180, 190) with an overtube extending from the bottom to the top is used,
wherein the height-adjustable immersion tube is formed of a height-adjustable inner tube (181, 191), which has an opening (182, 192) for removing one of the phases and an overtube extending from bottom to top, and the length of the overtube is defined by a lower end, which is arranged below the heavy phase (140), and an upper end, wherein the diameter of the overtube is adjusted to be larger than the diameter of the inner tube, such that an intermediate space is formed between the inner tube (181, 191) and the overtube, and the length of the overtube is selected so that the overtube projects beyond the opening (182, 192) of the inner tube (181, 191) and the upper end of the overtube is immersed in the light phase (120) or, if present, in the intermediate phase (130), such that the fluid of the phase in which the upper end of the overtube is immersed flows into the intermediate space and can exit through the opening (182, 192) in the inner tube (181, 191).

6. The method according to claim 4 or 5,
characterized in that
the length of the overtube (183, 193) extending from top to bottom and its diameter are selected in such a way that the entry of suspended matter, flocs and other solid or semi-solid components, into the intermediate space between the inner tube (181, 191) and the overtube (183, 193) is prevented by the overtube (183, 193);
or
the length of the overtube extending from bottom to top and its diameter are selected in such a way that the entry of suspended matter, flocs and other solid or semi-solid components into the intermediate space between the inner tube (181, 191) and the overtube is prevented by the overtube.

7. The method according to one of the preceding claims 1 to 6,
characterized in that
the probe(s) (160.2, 162.3, 162.4, 163.3, 163.4) for the light phase (120) is (are) positioned lower than the upper outlet (150.2) in the separating device (100) and the probe(s) (160.1, 162.1, 163.1) for the heavy phase (140) is (are) positioned higher than the lower outlet (150.1) in the separating device (100);
and/or
an immersion tube (180, 190) with or without an overtube (183, 193) is used,
wherein, when using an immersion tube (180, 190) without an overtube (183, 193), the opening (182, 192) of the immersion tube (180, 190) for removing the heavy phase (140) is located lower than the probe(s) (160.1, 162.1, 163.1) in the heavy phase (140) and wherein the opening (182, 192) of an immersion tube (180, 190) for removing the light phase (120) is located higher than the probe(s) (160.2, 162.3, 162.4, 163.3, 163.4) in the light phase (120); and
wherein, when using an immersion tube (180, 190) with an overtube (183, 193) extending from top to bottom, the lower end (183a, 193a) of the overtube (183, 193) is located lower than the probe(s) (160.1, 162.1, 163.1) in the heavy phase (140) for removing the heavy phase (140) when the heavy phase (140) is removed through the opening (182, 192) of the immersion tube (180, 190); and
wherein, when using an immersion tube with an overtube extending from the bottom to the top, when the light phase (120) is removed through the opening (182, 192) of the immersion tube, the upper end of the overtube for removing the light phase (120) is located higher than the probe(s) (160.2, 162.3, 162.4, 163.3, 163.4) in the light phase (120).

8. The method according to one of the preceding claims 1 to 7,
characterized in that
continuously measuring the electrical conductivity of the light phase (120) in the separating device (100) in step e. for one or more probes (160.2, 162.3, 162.4, 163.3, 163.4) in each case produces a value for electrical conductivity, which is compared with a target value range for the electrical conductivity of the light phase (120) and, if the target value range is exceeded, heavy phase (140) is removed, wherein the removal quantity, removal rate and/or removal duration for the heavy phase (140) are controlled in such a way that the measured conductivity of the light phase (120) is again in the target value range;
or
continuously measuring the electrical conductivity of the heavy phase (140) in the separating device (100) in step e. for one or more probes (160.1, 162.1, 163.1) in each case produces a value for electrical conductivity, which is compared with a target value range for the electrical conductivity of the heavy phase and, if the electrical conductivity falls below the target value range, less heavy phase (140) is removed, wherein the removal quantity, removal rate and/or removal duration for the heavy phase (140) are controlled in such a way that the measured conductivity of the light phase (120) is again in the target value range.

9. The method according to one of the preceding claims 1 to 8,
characterized in that
the electrical conductivity values are used to vary the position of the phase(s) and phase boundary interface(s) in continuous operation by monitoring and controlling one or more of the following:
the removal rate for the heavy phase being increased or decreased;
the removal rate for the light phase being increased or decreased;
the removal quantity for the heavy phase being increased or decreased;
the removal quantity for the light phase being increased or decreased;
the removal duration for the heavy phase being increased or decreased;
the removal duration for the light phase being increased or decreased;
the removal of the light or heavy phase being interrupted;
the feed quantity of the aqueous two-phase system flowing into the separating device (100), with or without the target product, being increased or decreased, wherein the aqueous two-phase system without the target product is a flushing solution;
the feed rate of the aqueous two-phase system flowing into the separating device, with or without a target product, being increased or decreased, wherein the aqueous two-phase system without a target product is a flushing solution;
and/or
the immersion tube being driven up or down to allow to flow out or suck away one or two phases and, if necessary, repositioned upward or downward;
wherein the one or more removal devices and/or means may be used to carry out the preceding measures:
gravity;
positive pressure;
negative pressure;
one or more pumps;
and/or
removal devices for controlled draining or interrupting the removal of phases at the outlet, selected from continuously adjustable valves with different degrees of opening, adjustable hose pinch clamps, adjustable shut-off valves or adjustable flow limiters.

10. The method according to one of the preceding claims 1 to 9,
characterized in that
for continuously measuring the electrical conductivity of the phases in the separating device (10, 100), two, three, four or more probes (160.1, 160.2, 162.1, 162.2, 162.3, 162.4, 163.1, 163.2, 163.3, 163.4) are each inserted into and used in an insertion opening (157.1, 157.2, 158.1, 158.2, 158.3, 158.4, 159.1, 159.2, 159.3, 159.4) in the wall (155) of the separating device (100), wherein the insertion openings (157.1, 157.2, 158.1, 158.2, 158.3, 158.4, 159.1, 159.2, 159.3, 159.4) have an arrangement that is selected from:
an axial arrangement of the insertion openings (157.1, 157.2), wherein the insertion openings (157.1, 157.2) are arranged on a straight connecting line;
a radial arrangement of the insertion openings (158.1, 158.2, 158.3, 158.4), wherein the insertion openings (158.1, 158.2, 158.3, 158.4) are arranged in an arc; or
a radially offset arrangement of the insertion openings (159.1, 159.2, 159.3, 159.4), wherein the insertion openings (159.1, 159.2, 159.3, 159.4) are arranged in an arc and additionally shifted in a preferred direction.

11. The method according to one of the preceding claims 1 to 10,
characterized in that
the mixing of the aqueous two-phase system is carried out by using active mixing and/or static mixing.

12. The method according to one of the preceding claims 1 to 11,
characterized in that at least one of the following conditions is satisfied:
the method is carried out continuously or discontinuously;
a cell culture, cell culture supernatant or cell lysate containing the target product is used as the starting solution;
an aqueous two-phase system is prepared with the starting solution, with which a phase ratio of the phase containing the target product to the counterphase of >1.1 is present;
a density ratio of light phase (20, 120) to heavy phase (40, 140) of at least 1.06 is used;
in the aqueous two-phase system, polyethylene glycol (PEG), polypropylene glycol (PPG), dextran, PEG-PPG-PEG copolymer (EOPO) or mixtures of these are used as the polymer or as one of the polymers;
in the aqueous two-phase system, polyethylene glycol with a molecular weight between 200 and 8000 g/mol is used as the polymer;
in the aqueous two-phase system, at least one polymer is used in a concentration of from 5 to 35 wt. %, based on the total quantity of aqueous two-phase system;
in the aqueous two-phase system, a phosphate salt, ammonium salt, potassium salts, acetate salts, sodium salts or citrate salt or mixtures of these are used as salt or as one of the salts;
instead of a salt, an acid selected from citric acid or acetic acid is used;
stabilizing components selected from amino acids, sugars or other stabilizing additives are added to the aqueous two-phase system;
the method is carried out continuously, but steps a. and/or d. are carried out semi-continuously.

13. The method according to one of the preceding claims 1 to 12,
characterized in that
instead of or in addition to the continuous measurement of the electrical conductivity, the turbidity in at least one of the phases in the separating device (10, 100) is continuously measured, wherein the turbidity can be carried out with one or more turbidity measuring devices in the form of one, two, three or more probes.

14. The method according to one of the preceding claims 1 to 13,
characterized in that
at the end of the extraction method for the residual discharge of a light phase (120) containing the target product, a flushing solution (115) is added to the separating device (100), which displaces the light phase (120) containing the target product from the separating device (100) and the latter can thus be removed through the outlet (150.2) arranged at the top,
and/or
at the end of the extraction method for the residual discharge of a light phase (120) containing the target product, no more heavy phase (140) is removed, the latter thereby accumulates and displaces the light phase (120) upward, such that the light phase (120) can be removed through the outlet (150.2) arranged at the top;
or
at the end of the extraction method for the residual discharge of a heavy phase (140) containing the target product, a flushing solution (115) is added to the separating device (100), which displaces the heavy phase (140) containing the target product from the separating device (100) and the latter can thus be removed through the outlet (150.1, 150.3) arranged at the bottom,
and/or
at the end of the extraction method for residual discharge of a heavy phase (140) containing the target product, no more light phase (120) is removed, the latter thereby accumulates and displaces the heavy phase (140) downward, such that the heavy phase (140) can be removed through the outlet (150.1, 150.3) arranged at the bottom.

15. A device for carrying out the method according to one of the claims 1 to 14, comprising
a separating device (10, 100) for separating the phases;
at least one outlet (50, 150.1, 150.2) in the separating device (10, 100);
at least one probe (60, 160.1, 160.2, 162.1, 162.2, 162.3, 162.4, 163.1, 163.2, 163.3, 163.4) for measuring electrical conductivity and/or at least one probe for measuring turbidity in at least one of the phases in the separating device (10, 100);
one or more removal device(s) and/or means for regulating and/or controlling the removal quantity, removal rate and/or removal duration of the light phase (20, 120), the heavy phase (40, 140) and, if present, the intermediate phase (30, 130), based on the measured values of electrical conductivity and/or based on the measured values of turbidity in at least one phase.

16. A use of the method according to one of the claims 1 to 14 or the device according to claim 15 for adjusting the position of the phases in a separating device (10, 100) and separating the phases based on the measured electrical conductivity values of at least one of the phases and/or based on the measured turbidity values of at least one of the phases.
